SEQUENCE LISTING (86 sequences)

SEQ ID NO: 1 - DNA, 1407 bp, Homo sapiens
atggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactga

SEQ ID NO: 2 - DNA, 1326 bp, Homo sapiens
atggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaa

SEQ ID NO: 3 - DNA, 1518 bp, Homo sapiens
atgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactag

SEQ ID NO: 4 - DNA, 1419 bp, Homo sapiens
atgggatcaaaaatgaatctcattgaacattcccatttacctaccacagatgaattttctttttctgaaaatttatttggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtga

SEQ ID NO: 5 - DNA, 1305 bp, Homo sapiens
ctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggcatcacaggtagctga

SEQ ID NO: 6 - DNA, 1344 bp, Homo sapiens
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 7 - DNA, 1383 bp, Homo sapiens
atgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtga

SEQ ID NO: 8 - DNA, 1047 bp, Homo sapiens
atggccagtagggaagatgagctgaggaactgtgtggtatgtggggaccaagccacaggctaccactttaatgcgctgacttgtgagggctgcaagggtttcttcaggagaacagtcagcaaaagcattggtcccacctgcccctttgctggaagctgtgaagtcagcaagactcagaggcgccactgcccagcctgcaggttgcagaagtgcttagatgctggcatgaggaaagacatgatactgtcggcagaagccctggcattgcggcgagcaaagcaggcccagcggcgggcacagcaaacacctgtgcaactgagtaaggagcaagaagagctgatccggacactcctgggggcccacacccgccacatgggcaccatgtttgaacagtttgtgcagtttaggcctccagctcatctgttcatccatcaccagcccttgcccaccctggcccctgtgctgcctctggtcacacacttcgcagacatcaacactttcatggtactgcaagtcatcaagtttactaaggacctgcccgtcttccgttccctgcccattgaagaccagatctcccttctcaagggagcagctgtggaaatctgtcacatcgtactcaataccactttctgtctccaaacacaaaacttcctctgcgggcctcttcgctacacaattgaagatggagcccgtgtggggttccaggtagagtttttggagttgctctttcacttccatggaacactacgaaaactgcagctccaagagcctgagtatgtgctcttggctgccatggccctcttctctcctgaccgacctggagttacccagagagatgagattgatcagctgcaagaggagatggcactgactctgcaaagctacatcaagggccagcagcgaaggccccgggatcggtttctgtatgcgaagttgctaggcctgctggctgagctccggagcattaatgaggcctacgggtaccaaatccagcacatccagggcctgtctgccatgatgccgctgctccaggagatctgcagctga

SEQ ID NO: 9 - protein, 5 aa, Artificial
  co-activator concensus interaction domain
LXXLL

SEQ ID NO: 10 - protein, 6 aa, Artificial
  Concensus co-repressor interaction domain
HIXXXX

SEQ ID NO: 11 - DNA, 4323 bp, Homo sapiens
atgagtggcctcggggacagttcatccgaccctgctaacccagactcacataagaggaaaggatcgccatgtgacacactggcatcaagcacggaaaagaggcgcagggagcaagaaaataaatatttagaagaactagctgagttactgtctgccaacattagtgacattgacagcttgagtgtaaaaccagacaaatgcaagattttgaagaaaacagtcgatcagatacagctaatgaagagaatggaacaagagaaatcaacaactgatgacgatgtacagaaatcagacatctcatcaagtagtcaaggagtgatagaaaaggaatccttgggaccccttcttttggaggctttggatggatttttctttgttgtgaactgtgaagggagaattgtatttgtgtcagagaatgtaaccagctacttaggttacaatcaggaggaattaatgaataccagcgtctacagcatactgcacgtgggggatcatgcagaatttgtgaagaatctgctaccaaaatcactagtaaatggagttccttggcctcaagaggcaacacgacgaaatagccatacctttaactgcaggatgctaattcaccctccagatgagccagggaccgagaaccaagaagcttgccagcgttatgaagtaatgcagtgtttcactgtgtcacagccaaaatcaattcaagaggatggagaagatttccagtcatgtctgatttgtattgcacggcgattacctcggcctccagctattacgggtgtagaatcctttatgaccaagcaagatactacaggtaaaatcatctctattgatactagttccctgagagctgctggcagaactggttgggaagatttagtgaggaagtgcatttatgcttttttccaacctcagggcagagaaccatcttatgccagacagctgttccaagaagtgatgactcgtggcactgcctccagcccctcctatagattcatattgaatgatgggacaatgcttagcgcccacaccaagtgtaaactttgctaccctcaaagtccagacatgcaacctttcatcatgggaattcatatcatcgacagggagcacagtgggctttctcctcaagatgacactaattctggaatgtcaattccccgagtaaatccctcggtcaatcctagtatctctccagctcatggtgtggctcgttcatccacattgccaccatccaacagcaacatggtatccaccagaataaaccgccagcagagctcagaccttcatagcagcagtcatagtaattctagcaacagccaaggaagtttcggatgctcacccggaagtcagattgtagccaatgttgccttaaaccaaggacaggccagttcacagagcagtaatccctctttaaacctcaataattctcctatggaaggtacaggaatatccctagcacagttcatgtctccaaggagacaggttacttctggattggcaacaaggcccaggatgccaaacaattcctttcctcctaatatttcgacattaagctctcccgttggcatgacaagtagtgcctgtaataataataaccgatcttattcaaacatcccagtaacatctttacagggtatgaatgaaggacccaataactccgttggcttctctgccagttctccagtcctcaggcagatgagctcacagaattcacctagcagattaaatatacaaccagcaaaagctgagtccaaagataacaaagagattgcctcaattttaaatgaaatgattcaatctgacaacagctctagtgatggcaaacctctggattcagggcttctgcataacaatgacagactttcagatggagacagtaaatactctcaaaccagtcacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagaacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaactccaaacctgagcctggatgatgtaaaggtgaaagtggaaaagaaagaacagatggatccatgtaatacaaacccaaccccaatgaccaaacccactcctgaggaaataaaactggaggcccagagccagtttacagctgaccttgaccagtttgatcagttactgcccacgctggagaaggcagcacagttgccaggcttatgtgagacagacaggatggatggtgcggtcaccagtgtaaccatcaaatcggagatcctgccagcttcacttcagtccgccactgccagacccacttccaggctaaatagattacctgagctggaattggaagcaattgataaccaatttggacaaccaggaacaggcgatcagattccatggacaaataatacagtgacagctataaatcagagtaaatcagaagaccagtgtattagctcacaattagatgagcttctctgtccacccacaacagtagaagggagaaatgatgagaaggctcttcttgaacagctggtatccttccttagtggcaaagatgaaactgagctagctgaactagacagagctctgggaattgacaaacttgttcaggggggtggattagatgtattatcagagagatttccaccacaacaagcaacgccacctttgatcatggaagaaagacccaacctttattcccagccttactcttctccttctcctactgccaatctccctagccctttccaaggcatggtcaggcaaaaaccttcactggggacgatgcctgttcaagtaacacctccccgaggtgctttttcacctggcatgggcatgcagcccaggcaaactctaaacagacctccggctgcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactaccagttcaaatggggaacccccgtcttcctcagggtgctccacagcaattcccctatccaccaaactatggtacaaatccaggaaccccacctgcttctaccagcccgttttcacaactagcagcaaatcctgaagcatccttggccaaccgcaacagcatggtgagcagaggcatgacaggaaacataggaggacagtttggcactggaatcaatcctcagatgcagcagaatgtcttccagtatccaggagcaggaatggttccccaaggtgaggccaactttgctccatctctaagccctgggagctccatggtgccgatgccaatccctcctcctcagagttctctgctccagcaaactccacctgcctccgggtatcagtcaccagacatgaaggcctggcagcaaggagcgataggaaacaacaatgtgttcagtcaagctgtccagaaccagcccacgcctgcacagccaggagtatacaacaacatgagcatcaccgtttccatggcaggtggaaatacgaatgttcagaacatgaacccaatgatggcccagatgcagatgagctctttgcagatgccaggaatgaacactgtgtgccctgagcagataaatgatcccgcactgagacacacaggcctctactgcaaccagctctcatccactgaccttctcaaaacagaagcagatggaacccaggtgcaacaggttcaggtgtttgctgacgtccagtgtacagtgaatctggtaggcggggacccttacctgaaccagcctggtccactgggaactcaaaagcccacgtcaggaccacagaccccccaggcccagcagaagagcctccttcagcagctactgactgaataa

SEQ ID NO: 12 - DNA, 4746 bp, Homo sapiens
atgaaagctcagggggaaaccgaggagtcagaaaagctgagtaagatgagttctctcctggaacggctccatgcaaaatttaaccaaaatagaccctggagtgaaaccattaagcttgtgcgtcaagtcatggagaagagggttgtgatgagttctggagggcatcaacatttggtcagctgtttggagacattgcagaaggctctcaaagtaacatctttaccagcaatgactgatcgtttggagtccatagcaagacagaatggactgggctctcatctcagtgccagtggcactgaatgttacatcacgtcagatatgttctatgtggaagtgcagttagatcctgcaggacagctttgtgatgtaaaagtggctcaccatggggagaatcctgtgagctgtccggagcttgtacagcagctaagggaaaaaaattttgatgaattttctaagcaccttaagggccttgttaatctgtataaccttccaggggacaacaaactgaagactaaaatgtacttggctctccaatccttagaacaagatctttctaaaatggcaattatgtactggaaagcaactaatgctggtcccttggataagattcttcatggaagtgttggctatctcacaccaaggagtgggggtcatttaatgaacctgaagtactatgtctctccttctgacctactggatgacaagactgcatctcccatcattttgcatgagaataatgtttctcgatctttgggcatgaatgcatcagtgacaattgaaggaacatctgctgtgtacaaactcccaattgcaccattaattatggggtcacatccagttgacaataaatggaccccttccttctcctcaatcaccagtgccaacagtgttgatcttcctgcctgtttcttcttgaaatttccccagccaatcccagtatctagagcatttgttcagaaactgcagaactgcacaggaattccattgtttgaaactcaaccaacttatgcacccctgtatgaactgatcactcagtttgagctatcaaaggaccctgaccccatacctttgaatcacaacatgagattttatgctgctcttcctggtcagcagcactgctatttcctcaacaaggatgctcctcttccagatggccgaagtctacagggaacccttgttagcaaaatcacctttcagcaccctggccgagttcctcttatcctaaatctgatcagacaccaagtggcctataacaccctcattggaagctgtgtcaaaagaactattctgaaagaagattctcctgggcttctccaatttgaagtgtgtcctctctcagagtctcgtttcagcgtatcttttcagcaccctgtgaatgactccctggtgtgtgtggtaatggatgtgcaggactcaacacatgtgagctgtaaactctacaaagggctgtcggatgcactgatctgcacagatgacttcattgccaaagttgttcaaagatgtatgtccatccctgtgacgatgagggctattcggaggaaagctgaaaccattcaagccgacaccccagcactgtccctcattgcagagacagttgaagacatggtgaaaaagaacctgcccccggctagcagcccagggtatggcatgaccacaggcaacaacccaatgagtggtaccactacaccaaccaacacctttccggggggtcccattaccaccttgtttaatatgagcatgagcatcaaagatcggcatgagtcggtgggccatggggaggacttcagcaaggtgtctcagaacccaattcttaccagtttgttgcaaatcacagggaacggggggtctaccattggctcgagtccgacccctcctcatcacacgccgccacctgtctcttcgatggccggcaacaccaagaaccacccgatgctcatgaaccttcttaaagataatcctgcccaggatttctcaaccctttatggaagcagccctttagaaaggcagaactcctcttccggctcaccccgcatggaaatatgctcggggagcaacaagaccaagaaaaagaagtcatcaagattaccacctgagaaaccaaagcaccagactgaagatgactttcagagggagctattttcaatggatgttgactcacagaaccctatctttgatgtcaacatgacagctgacacgctggatacgccacacatcactccagctccaagccagtgtagcactcccccaacaacttacccacaaccagtacctcacccccaacccagtattcaaaggatggtccgactatccagttcagacagcattggcccagatgtaactgacatcctttcagacattgcagaagaagcttctaaacttcccagcactagtgatgattgcccagccattggcacccctcttcgagattcttcaagctctgggcattctcagagtaccctgtttgactctgatgtctttcaaactaacaataatgaaaatccatacactgatccagctgatcttattgcagatgctgctggaagccccagtagtgactctcctaccaatcatttttttcatgatggagtagatttcaatcctgatttattgaacagccagagccaaagtggttttggagaagaatattttgatgaaagcagccaaagtggggataatgatgatttcaaaggatttgcatctcaggcactaaatactttgggggtgccaatgcttggaggtgataatggggagaccaagtttaagggcaataaccaagccgacacagttgatttcagtattatttcagtagccggcaaagctttagctcctgcagatcttatggagcatcacagtggtagtcagggtcctttactgaccactggggacttagggaaagaaaagactcaaaagagggtaaaggaaggcaatggcaccagtaatagtactctctcggggcccggattagacagcaaaccagggaagcgcagtcggaccccttctaatgatgggaaaagcaaagataagcctccaaagcggaagaaggcagacactgagggaaagtctccatctcatagttcttctaacagaccttttaccccacctaccagtacaggtggatctaaatcgccaggcagtgcaggaagatctcagactcccccaggtgttgccacaccacccattcccaaaatcactattcagattcctaagggaacagtgatggtgggcaagccttcctctcacagtcagtataccagcagtggttctgtgtcttcctcaggcagcaaaagccaccatagccattcttcctcctcttcctcatctgcttccacctcagggaagatgaaaagcagtaaatcagaaggttcatcaagttccaagttaagtagcagtatgtattctagccaggggtcttctggatctagccagtccaaaaattcatcccagtctggggggaagccaggctcctctcccataaccaagcatggactgagcagtggctctagcagcaccaagatgaaacctcaaggaaagccatcatcacttatgaatccttctttaagtaaaccaaacatatccccttctcattcaaggccacctggaggctctgacaagcttgcctctccaatgaagcctgttcctggaactcctccatcctctaaagccaagtcccctatcagttcaggttctggtggttctcatatgtctggaactagttcaagctctggcatgaagtcatcttcagggttaggatcctcaggctcgttgtcccagaaaactcccccatcatctaattcctgtacggcatcttcctcctccttttcctcaagtggctcttccatgtcatcctctcagaaccagcatgggagttctaaaggaaaatctcccagcagaaacaagaagccgtccttgacagctgtcatagataaactgaagcatggggttgtcaccagtggccctgggggtgaagacccactggacggccagatgggggtgagcacaaattcttccagccatcctatgtcctccaaacataacatgtcaggaggagagtttcagggcaagcgtgagaaaagtgataaagacaaatcaaaggtttccacctccgggagttcagtggattcttctaagaagacctcagagtcaaaaaatgtggggagcacaggtgtggcaaaaattatcatcagtaagcatgatggaggctcccctagcattaaagccaaagtgactttgcagaaacctggggaaagtagtggagaagggcttaggcctcaaatggcttcttctaaaaactatggctctccactcatcagtggttccactccaaagcatgagcgtggctctcccagccatagtaagtcaccagcatataccccccagaatctggacagtgaaagtgagtcaggctcctccatagcagagaaatcttatcagaatagtcccagctcagacgatggtatccgaccacttccagaatacagcacagagaaacataagaagcacaaaaaggaaaagaagaaagtaaaagacaaagatagggaccgagaccgggacaaagaccgagacaagaaaaaatctcatagcatcaagccagagagttggtccaaatcacccatctcttcagaccagtccttgtctatgacaagtaacacaatcttatctgcagacagaccctcaaggctcagcccagactttatgattggggaggaagatgatgatcttatggatgtggccctgattgggaattag

SEQ ID NO: 13 - DNA, 4395 bp, Homo sapiens
atgagtgggatgggagaaaatacctctgacccctccagggcagagacaagaaagcgcaaggaatgtcctgaccaacttggacccagccccaaaaggaacactgaaaaacgtaatcgtgaacaggaaaataaatatatagaagaacttgcagagttgatttttgcaaattttaatgatatagacaactttaacttcaaacctgacaaatgtgcaatcttaaaagaaactgtgaagcaaattcgtcagatcaaagaacaagagaaagcagcagctgccaacatagatgaagtgcagaagtcagatgtatcctctacagggcagggtgtcatcgacaaggatgcgctggggcctatgatgcttgaggcccttgatgggttcttctttgtagtgaacctggaaggcaacgttgtgtttgtgtcagagaatgtgacacagtatctaaggtataaccaagaagagctgatgaacaaaagtgtatatagcatcttgcatgttggggaccacacggaatttgtcaaaaacctgctgccaaagtctatagtaaatgggggatcttggtctggcgaacctccgaggcggaacagccataccttcaattgtcggatgctggtaaaacctttacctgattcagaagaggagggtcatgataaccaggaagctcatcagaaatatgaaactatgcagtgcttcgctgtctctcaaccaaagtccatcaaagaagaaggagaagatttgcagtcctgcttgatttgcgtggcaagaagagttcccatgaaggaaagaccagttcttccctcatcagaaagttttactactcgccaggatctccaaggcaagatcacgtctctggataccagcaccatgagagcagccatgaaaccaggctgggaggacctggtaagaaggtgtattcagaagttccatgcgcagcatgaaggagaatctgtgtcctatgctaagaggcatcatcatgaagtactgagacaaggattggcattcagtcaaatctatcgtttttccttgtctgatggcactcttgttgctgcacaaacgaagagcaaactcatccgttctcagactactaatgaacctcaacttgtaatatctttacatatgcttcacagagagcagaatgtgtgtgtgatgaatccggatctgactggacaaacgatggggaagccactgaatccaattagctctaacagccctgcccatcaggccctgtgcagtgggaacccaggtcaggacatgaccctcagtagcaatataaattttcccataaatggcccaaaggaacaaatgggcatgcccatgggcaggtttggtggttctgggggaatgaaccatgtgtcaggcatgcaagcaaccactcctcagggtagtaactatgcactcaaaatgaacagcccctcacaaagcagccctggcatgaatccaggacagcccacctccatgctttcaccaaggcatcgcatgagccctggagtggctggcagccctcgaatcccacccagtcagttttcccctgcaggaagcttgcattcccctgtgggagtttgcagcagcacaggaaatagccatagttataccaacagctccctcaatgcacttcaggccctcagcgaggggcacggggtctcattagggtcatcgttggcttcaccagacctaaaaatgggcaatttgcaaaactccccagttaatatgaatcctcccccactcagcaagatgggaagcttggactcaaaagactgttttggactatatggggagccctctgaaggtacaactggacaagcagagagcagctgccatcctggagagcaaaaggaaacaaatgaccccaacctgcccccggccgtgagcagtgagagagctgacgggcagagcagactgcatgacagcaaagggcagaccaaactcctgcagctgctgaccaccaaatctgatcagatggagccctcgcccttagccagctctttgtcggatacaaacaaagactccacaggtagcttgcctggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtgactattaaacaagagccggtgagccccaagaagaaagagaatgcactacttcgctatttgctagataaagatgatactaaagatattggtttaccagaaataacccccaaacttgagagactggacagtaagacagatcctgccagtaacacaaaattaatagcaatgaaaactgagaaggaggagatgagctttgagcctggtgaccagcctggcagtgagctggacaacttggaggagattttggatgatttgcagaatagtcaattaccacagcttttcccagacacgaggccaggcgcccctgctggatcagttgacaagcaagccatcatcaatgacctcatgcaactcacagctgaaaacagccctgtcacacctgttggagcccagaaaacagcactgcgaatttcacagagcacttttaataacccacgaccagggcaactgggcaggttattgccaaaccagaatttaccacttgacatcacattgcaaagcccaactggtgctggacctttcccaccaatcagaaacagtagtccctactcagtgatacctcagccaggaatgatgggtaatcaagggatgataggaaaccaaggaaatttagggaacagtagcacaggaatgattggtaacagtgcttctcggcctactatgccatctggagaatgggcaccgcagagttcggctgtgagagtcacctgtgctgctaccaccagtgccatgaaccggccagtccaaggaggtatgattcggaacccagcagccagcatccccatgaggcccagcagccagcctggccaaagacagacgcttcagtctcaggtcatgaatatagggccatctgaattagagatgaacatggggggacctcagtatagccaacaacaagctcctccaaatcagactgccccatggcctgaaagcatcctgcctatagaccaggcgtcttttgccagccaaaacaggcagccatttggcagttctccagatgacttgctatgtccacatcctgcagctgagtctccgagtgatgagggagctctcctggaccagctgtatctggccttgcggaattttgatggcctggaggagattgatagagccttaggaatacccgaactggtcagccagagccaagcagtagatccagaacagttctcaagtcaggattccaacatcatgctggagcagaaggcgcccgttttcccacagcagtatgcatctcaggcacaaatggcccagggtagctattctcccatgcaagatccaaactttcacaccatgggacagcggcctagttatgccacactccgtatgcagcccagaccgggcctcaggcccacgggcctagtgcagaaccagccaaatcaactaagacttcaacttcagcatcgcctccaagcacagcagaatcgccagccacttatgaatcaaatcagcaatgtttccaatgtgaacttgactctgaggcctggagtaccaacacaggcacctattaatgcacagatgctggcccagagacagagggaaatcctgaaccagcatcttcgacagagacaaatgcatcagcaacagcaagttcagcaacgaactttgatgatgagaggacaagggttgaatatgacaccaagcatggtggctcctagtggtatgccagcaactatgagcaaccctcggattccccaggcaaatgcacagcagtttccatttcctccaaactacggaataagtcagcaacctgatccaggctttactggggctacgactccccagagcccacttatgtcaccccgaatggcacatacacagagtcccatgatgcaacagtctcaggccaacccagcctatcaggccccctccgacataaatggatgggcgcaggggaacatgggcggaaacagcatgttttcccagcagtccccaccacactttgggcagcaagcaaacaccagcatgtacagtaacaacatgaacatcaatgtgtccatggcgaccaacacaggtggcatgagcagcatgaaccagatgacaggacagatcagcatgacctcagtgacctccgtgcctacgtcagggctgtcctccatgggtcccgagcaggttaatgatcctgctctgaggggaggcaacctgttcccaaaccagctgcctggaatggatatgattaagcaggagggagacacaacacggaaatattgctga

SEQ ID NO: 14 - DNA, 7554 bp, Homo sapiens
atgtcgggctccacacagcttgtggcacagacgtggagggccactgagccccgctacccgccccacagcctttcctacccagtgcagatcgcccggacgcacacggacgtcgggctcctggagtaccagcaccactcccgcgactatgcctcccacctgtcgccgggctccatcatccagccccagcggcggaggccctccctgctgtctgagttccagcccgggaatgaacggtcccaggagctccacctgcggccagagtcccactcatacctgcccgagctggggaagtcagagatggagttcattgaaagcaagcgccctcggctagagctgctgcctgaccccctgctgcgaccgtcacccctgctggccacgggccagcctgcgggatctgaagacctcaccaaggaccgtagcctgacgggcaagctggaaccggtgtctccccccagccccccgcacactgaccctgagctggagctggtgccgccacggctgtccaaggaggagctgatccagaacatggaccgcgtggaccgagagatcaccatggtagagcagcagatctctaagctgaagaagaagcagcaacagctggaggaggaggctgccaagccgcccgagcctgagaagcccgtgtcaccgccgcccatcgagtcgaagcaccgcagcctggtgcagatcatctacgacgagaaccggaagaaggctgaagctgcacatcggattctggaaggcctggggccccaggtggagctgccgctgtacaaccagccctccgacacccggcagtatcatgagaacatcaaaataaaccaggcgatgcggaagaagctaatcttgtacttcaagaggaggaatcacgctcggaaacaatggaagcagaagttctgccagcgctatgaccagctcatggaggccttggaaaaaaaggtggagcgcatcgaaaacaacccgcgccggcgggccaaggagagcaaggtgcgcgagtactacgaaaagcagttccctgagatccgcaagcagcgcgagctgcaggagcgcatgcagagcagggtgggccagcggggcagtgggctgtccatgtcggccgcccgcagcgagcacgaggtgtcagagatcatcgatggcctctcagagcaggagaacctggagaagcagatgcgccagctggccgtgatcccgcccatgctgtacgacgctgaccagcagcgcatcaagttcatcaacatgaacgggcttatggccgaccccatgaaggtgtacaaagaccgccaggtcatgaacatgtggagtgagcaggagaaggagaccttccgggagaagttcatgcagcatcccaagaactttggcctgatcgcatcattcctggagaggaagacagtggctgagtgcgtcctctattactacctgactaagaagaatgagaactataagagcctggtgagacggagctatcggcgccgcggcaagagccagcagcaacaacagcagcagcagcagcagcagcagcagcagcagcagcagcccatgccccgcagcagccaggaggagaaagatgagaaggagaaggaaaaggaggcggagaaggaggaggagaagccggaggtggagaacgacaaggaagacctcctcaaggagaagacagacgacacctcaggggaggacaacgacgagaaggaggctgtggcctccaaaggccgcaaaactgccaacagccagggaagacgcaaaggccgcatcacccgctcaatggctaatgaggccaacagcgaggaggccatcaccccccagcagagcgccgagctggcctccatggagctgaatgagagttctcgctggacagaagaagaaatggaaacagccaagaaaggtctcctggaacacggccgcaactggtcggccatcgcccggatggtgggctccaagactgtgtcgcagtgtaagaacttctacttcaactacaagaagaggcagaacctcgatgagatcttgcagcagcacaagctgaagatggagaaggagaggaacgcgcggaggaagaagaagaaagcgccggcggcggccagcgaggaggctgcattcccgcccgtggtggaggatgaggagatggaggcgtcgggcgtgagcggaaatgaggaggagatggtggaggaggctgaagccttacatgcctctgggaatgaggtgcccagaggggaatgcagtggcccagccactgtcaacaacagctcagacaccgagagcatcccctctcctcacactgaggccgccaaggacacagggcagaatgggcccaagcccccagccaccctgggcgccgacgggccacccccaggcccacccaccccaccacggaggacatcccgggcccccattgagcccaccccggcctctgaagccaccggagcccctacgcccccaccagcacccccatcgccctctgcacctcctcctgtggtccccaaggaggagaaggaggaggagaccgcagcagcgcccccagtggaggagggggaggagcagaagccccccgcggctgaggagctggcagtggacacagggaaggccgaggagcccgtcaagagcgagtgcacggaggaagccgaggaggggccggccaagggcaaggacgcggaggccgctgaggccacggccgagggggcgctcaaggcagagaagaaggagggcgggagcggcagggccaccactgccaagagctcgggcgccccccaggacagcgactccagtgctacctgcagtgcagacgaggtggatgaggccgagggcggcgacaagaaccggctgctgtccccaaggcccagcctcctcaccccgactggcgacccccgggccaatgcctcaccccagaagccactggacctgaagcagctgaagcagcgagcggctgccatcccccccatccaggtcaccaaagtccatgagcccccccgggaggacgcagctcccaccaagccagctcccccagccccaccgccaccgcaaaacctgcagccggagagcgacgcccctcagcagcctggcagcagcccccggggcaagagcaggagcccggcaccccccgccgacaaggaggccttcgcagccgaggcccagaagctgcctggggaccccccttgctggacttccggcctgcccttccccgtgcccccccgtgaggtgatcaaggcctccccgcatgccccggacccctcagccttctcctacgctccacctggtcacccactgcccctgggcctccatgacactgcccggcccgtcctgccgcgcccacccaccatctccaacccgcctcccctcatctcctctgccaagcaccccagcgtcctcgagaggcaaataggtgccatctcccaaggaatgtcggtccagctccacgtcccgtactcagagcatgccaaggccccggtgggccctgtcaccatggggctgcccctgcccatggaccccaaaaagctggcacccttcagcggagtgaagcaggagcagctgtccccacggggccaggctgggccaccggagagcctgggggtgcccacagcccaggaggcgtccgtgctgagagggacagctctgggctcagttccgggcggaagcatcaccaaaggcattcccagcacacgggtgccctcggacagcgccatcacataccgcggctccatcacccacggcacgccagctgacgtcctgtacaagggcaccatcaccaggatcatcggcgaggacagcccgagtcgcttggaccgcggccgggaggacagcctgcccaagggccacgtcatctacgaaggcaagaagggccacgtcttgtcctatgagggtggcatgtctgtgacccagtgctccaaggaggacggcagaagcagctcaggacccccccatgagacggccgcccccaagcgcacctatgacatgatggagggccgcgtgggcagagccatctcctcagccagcatcgaaggtctcatgggccgtgccatcccgccggagcgacacagcccccaccacctcaaagagcagcaccacatccgcgggtccatcacacaagggatccctcggtcctacgtggaggcacaggaggactacctgcgtcgggaggccaagctcctaaagcgggagggcacgcctccgcccccaccgccctcacgggacctgaccgaggcctacaagacgcaggccctgggccccctgaagctgaagccggcccatgagggcctggtggccacggtgaaggaggcgggccgctccatccatgagatcccgcgcgaggagctgcggcacacgcccgagctgcccctggccccgcggccgctcaaggagggctccatcacgcagggcaccccgctcaagtacgacaccggcgcgtccaccactggctccaaaaagcacgacgtacgctccctcatcggcagccccggccggacgttcccacccgtgcacccgctggatgtgatggccgacgcccgggcactggaacgtgcctgctacgaggagagcctgaagagccggccagggaccgccagcagctcggggggctccattgcgcgcggcgccccggtcattgtgcctgagctgggtaagccgcggcagagccccctgacctatgaggaccacggggcaccctttgccggccacctcccacgaggttcgcccgtgaccatgcgggagcccacgccgcgcctgcaggagggcagcctttcgtccagcaaggcatcccaggaccgaaagctgacgtcgacgcctcgtgagatcgccaagtccccgcacagcaccgtgcccgagcaccacccacaccccatctcgccctatgagcacctgcttcggggcgtgagtggcgtggacctgtatcgcagccacatccccctggccttcgaccccacctccataccccgcggcatccctctggacgcagccgctgcctactacctgccccgacacctggcccccaaccccacctacccgcacctgtacccaccctacctcatccgcggctaccccgacacggcggcgctggagaaccggcagaccatcatcaatgactacatcacctcgcagcagatgcaccacaacacggccaccgccatggcccagcgagctgatatgctgaggggcctctcgccccgcgagtcctcgctggcactcaactacgctgcgggtccccgaggcatcatcgacctgtcccaagtgccacacctgcctgtgctcgtgcccccgacaccaggcaccccagccaccgccatggaccgccttgcctacctccccaccgcgccccagcccttcagcagccgccacagcagctccccactctccccaggaggtccaacacacttgacaaaaccaaccaccacgtcctcgtccgagcgggagcgagaccgggatcgagagcgggaccgggatcgggagcgggaaaagtccatcctcacgtccaccacgacggtggagcacgcacccatctggagacctggtacagagcagagcagcggcagcagcggcagcagcggcgggggtgggggcagcagcagccgccccgcctcccactcccatgcccaccagcactcgcccatctcccctcggacccaggatgccctccagcagagacccagtgtgcttcacaacacaggcatgaagggtatcatcaccgctgtggagcccagcaagcccacggtcctgaggtccacctccacctcctcacccgttcgcccagctgccacattcccacctgccacccactgcccactgggcggcaccctcgatggggtctaccctaccctcatggagcccgtcttgctgcccaaggaggccccccgggtcgcccggccagagcggccccgagcagacaccggccatgccttcctcgccaagcccccagcccgctccgggctggagcccgcctcctcccccagcaagggctcggagccccggcccctagtgcctcctgtctctggccacgccaccatcgcccgcacccctgcgaagaacctcgcacctcaccacgccagcccggacccgccggcgccacctgcctcggcctcggacccgcaccgggaaaagactcaaagtaaacccttttccatccaggaactggaactccgttctctgggttaccacggcagcagctacagccccgaaggggtggagcccgtcagccctgtgagctcacccagtctgacccacgacaaggggctccccaagcacctggaagagctcgacaagagccacctggagggggagctgcggcccaagcagccaggccccgtgaagcttggcggggaggccgcccacctcccacacctgcggccgctgcctgagagccagccctcgtccagcccgctgctccagaccgccccaggggtcaaaggtcaccagcgggtggtcaccctggcccagcacatcagtgaggtcatcacacaggactacacccggcaccacccacagcagctcagcgcacccctgcccgcccccctctactccttccctggggccagctgccccgtcctggacctccgccgcccacccagtgacctctacctcccgcccccggaccatggtgccccggcccgtggctccccccacagcgaagggggcaagaggtctccagagccaaacaagacgtcggtcttgggtggtggtgaggacggtattgaacctgtgtccccaccggagggcatgacggagccagggcactcccggagtgctgtgtacccgctgctgtaccgggatggggaacagacggagcccagcaggatgggctccaagtctccaggcaacaccagccagccgccagccttcttcagcaagctgaccgagagcaactccgccatggtcaagtccaagaagcaagagatcaacaagaagctgaacacccacaaccggaatgagcctgaatacaatatcagccagcctgggacggagatcttcaatatgcccgccatcaccggaacaggccttatgacctatagaagccaggcggtgcaggaacatgccagcaccaacatggggctggaggccataattagaaaggcactcatgggtaaatatgaccagtgggaagagtccccgccgctcagcgccaatgcttttaaccctctgaatgccagtgccagcctgcccgctgctatgcccataaccgctgctgacggacggagtgaccacacactcacctcgccaggtggcggcgggaaggccaaggtctctggcagacccagcagccgaaaagccaagtccccggccccgggcctggcatctggggaccggccaccctctgtctcctcagtgcactcggagggagactgcaaccgccggacgccgctcaccaaccgcgtgtgggaggacaggccctcgtccgcaggttccacgccattcccctacaaccccctgatcatgcggctgcaggcgggtgtcatggcttccccacccccaccgggcctccccgcgggcagcgggcccctcgctggcccccaccacgcctgggacgaggagcccaagccactgctctgctcgcagtacgagacactctccgacagcgagtga

SEQ ID NO: 15 - DNA, 2745 bp, Homo sapiens
atgtcaagttcagtttatcctcccaaccaaggagcattcagcacagaacaaagtcgttctcctcctcactctgtaaagtatacgtttcccagcacccaccaccagcaggatccagcattcggaggcaaacatgaagctccatcctctccaatttcggggcaaccatgtggagatgatcaaaatgcttcaccttcaaaactctcaaaggaagagttaatacagagtatggatcgtgtagatcgagaaattgcaaaagtagaacagcagatccttaaactgaaaaagaaacaacaacagcttgaagaagaggcagctaaacctcctgagcctgagaagcccgtgtcccctcctcctgtggagcagaaacaccgcagtattgtccaaattatttatgatgagaatcggaaaaaagcagaagaagctcataaaatttttgaaggtcttggcccaaaagttgaactgccactgtataaccagccatcagataccaaggtgtaccatgagaacatcaagacaaaccaggtgatgaggaaaaaactcattttattttttaaaagaagaaatcatgcaagaaaacaaagggaacaaaaaatctgccagcgttatgatcagctcatggaggcatgggagaaaaaagtggacagaatagaaaataatcctcggaggaaagctaaagaaagcaaaacaagggaatactatgaaaagcagtttccagaaattcgaaaacaaagagaacagcaagaaagatttcagcgagttgggcagaggggagctggtctttcagccaccattgctaggagtgagcatgagatttctgaaattattgatgggctctctgagcaggaaagtaatgagaaacaaatgcggcagctctctgtgattccacctatgatgtttgatgcagaacaaagacgagtcaagttcattaacatgaatgggcttatggaggaccctatgaaagtgtataaagataggcagtttatgaatgtttggactgaccatgaaaaggagatctttaaggacaagtttatccagcatccaaaaaactttggactaattgcatcatacttggagaggaagagtgttcctgattgtgttttgtattactatttaaccaagaaaaatgagaattataaagccctcgtcagaaggaattatgggaaacgcagaggcagaaaccagcaaattgctcgaccctcgcaagaagaaaaagtagaagaaaaagaagaggataaagcagaaaaaacagaaaaaaaagaagaagaaaagaaagatgaagaggaaaaagatgaaaaagaagactccaaagaaaataccaaggaaaaggacaagatagatggtacagcagaagaaactgaggaaagagagcaagccacaccccgggggcgaaagactgccaacagtcagggccgccgtaagggccggatcaccaggtccatgacaaacgaagctgcagctgccagtgctgcagccgcagcggctactgaagagcccccaccacctctgccaccgccaccagaacccatttctacagagcctgtggagacctctcgatggacagaagaagaaatggaagttgctaaaaaaggtctagtagaacatggtcgtaactgggcagcaattgctaaaatggtgggaacgaaaagtgaagctcaatgtaaaaacttctattttaactataaaaggcgacacaatcttgacaacctcttacagcagcataaacagaaaacttcacgaaaacctcgtgaagagcgagatgtgtctcaatgtgaaagtgtcgcttccactgtttctgctcaggaggatgaagatattgaagcctccaatgaagaagaaaatccagaagacagcgaaggtgcagaaaatagttctgatacagaaagtgctccttctccttcaccagttgaagctgtcaagcccagcgaggacagtcctgaaaatgctacttctcgaggaaacacagaacctgcggttgagcttgagcccaccacggaaactgcacccagtacatctccctccttagcagttccaagtacaaaaccagctgaagatgaaagtgtggagacccaggtgaatgacagcatcagtgctgagacagcagagcagatggatgtagatcagcaggagcacagtgctgaagagggttctgtttgtgatcccccacccgctaccaaagctgactctgtggacgttgaagtgagggtgccagaaaaccatgcatctaaagttgaaggtgataataccaaagaaagagacttggatagagccagtgagaaggtggaacctagagatgaagatttggtggtagctcagcaaataaatgcccaaaggcccgagccccagtcagacaatgattccagtgccacgtgcagcgctgatgaggatgtggatggagagccagagaggcagagaatgtttcctatggactcaaagccttcactgttaaaccccactggatctatactcgtctcatctccgttaaaaccaaatccactggatctgccacagcttcagcatcgagctgctgttatcccaccaatggtatcctgcaccccatgtaacataccaattggaaccccagtgagcggctatgctctctaccagcgacacattaaagcaatgcatgagtcagcactcctggaggagcagcggcagagacaagaacagatagatttggaatgtagaagttctacaagtccatgtggcacatccaagagtccaaacagagagtgggaaggtaggtag

SEQ ID NO: 16 - protein, 20 aa, Artificial
  Concensus zinc finger motif
XXCXCXXXXXXXXXXXXHXX

SEQ ID NO: 17 - DNA, 450 bp, Saccharomyces cerevisiae
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgattgaattc

SEQ ID NO: 18 - DNA, 606 bp, Escherichia coli
atgaaagctctgaccgctcgtcagcaggaagttttcgacctgatccgtgaccacatctcccagaccggtatgccgccgacccgtgctgaaatcgctcagcgtctgggtttccgttccccgaacgctgctgaagaacacctgaaagctctggctcgtaaaggtgttatcgaaatcgtttccggtgcttcccgtggtatccgtctgctgcaggaagaagaagaaggtctgccgctggttggtcgtgttgctgctggtgaaccgctgctggctcagcagcacatcgaaggtcactaccaggttgacccgtccctgttcaaaccgaacgctgacttcctgctgcgtgtttccggtatgtccatgaaagacatcggtatcatggacggtgacctgctggctgttcacaaaacccaggacgttcgtaacggtcaggttgttgttgctcgtatcgacgacgaagttaccgttaaacgtctgaaaaaacagggtaacaaagttgaactgctgccggaaaactccgaattcaaaccgatcgttgttgacctgcgtcagcagtccttcaccatcgaaggtctggctgttggtgttatccgtaacggtgactggctg

SEQ ID NO: 19 - DNA, 387 bp, herpes simplex virus 7
agcagaggcagaaccagaaacaactacggctctaccatcgagggcttgctcgacctcccagacgacgacgacgctccagccgaagcaggtctcgttgctccaagaatgtctttcctctccgctggacagagaccaagaagactttctaccaccgctccaatcaccgacgtttctttggttgacgaattgagattggacggcgaggaggttgacatgaccccagccgacgccttggacgacttcgacttggagatgttgggtgacgttgagtccccatccccaggcatgacccacgacccagtttcctacggcgctttggacgttgacgacttcgagtttgagcagatgttcaccgacgccttgggcatcgacgacttcggtggctag

SEQ ID NO: 20 - DNA, 2612 bp, Human immunodeficiency virus type 1
atggagccagtagatcctagcctagagccctggaagcatccaggaagtcagcctaagactgcttgtaccacttgctattgtaaaaagtgttgccttcattgccaagtctgtttcataacaaaaggcttaggcatctcctatggcaggaagaagcggagacagcgacgaagagctcctcaagacagtcagactcatcaagcttctctatcaaagcagtaagtagtgcatgtaatgcaatctttatacatattagcaatagtagcattagtagtagcaataataatagcaatagttgtgtggtccatagtactcatagaatataggaaaatattaagacaaagaaaaatagataggttaattgatagaataagagagagagcagaagacagtggcaatgagagtgaaggggatcaggaagagttatcagtacttgtggaaagggggcaccttgctccttgggatattaatgatctgtagtgctgtagaaaagttgtgggtcacagtctattatggggtacctgtgtggaaagaagcaaccaccactctattttgtgcatcagatgctaaagcatatgatacagaggtacataatgtttgggccacacatgcctgtgtacccacagaccccaacccacaagaagtagtattggaaaatgtaacagaacattttaacatgtggaaaaataacatggtagaacagatgcaggaggatataatcagtttatgggatcaaagcctaaagccatgtgtaaaattaaccccactctgtgttactttaaattgcaaggatgtgaatgctactaataccactaatgatagcgagggaacgatggagagaggagaaataaaaaactgctctttcaatatcaccacaagcataagagatgaggtgcagaaagaatatgctcttttttataaacttgatgtagtaccaatagataataataataccagctataggttgataagttgtgacacctcagtcattacacaggcctgtccaaagatatcctttgagccaattcccatacattattgtgccccggctggttttgcgattctaaagtgtaatgataagacgttcaatggaaaaggaccatgtaaaaatgtcagcacagtacaatgtacacatggaattaggccagtagtatcaactcaactgctgctaaatggcagtctagcagaagaagaggtagtaattagatctgacaatttcacgaacaatgctaaaaccataatagtacagctgaaagaatctgtagaaattaattgtacaagacccaacaacaatacaagaaaaagtatacatataggaccagggagagcattttatactacaggagaaataataggagatataagacaagcacattgtaacattagtagagcaaaatggaatgacactttaaaacagatagttataaaattaagagaacaatttgagaataaaacaatagtctttaatcactcctcaggaggggacccagaaattgtaatgcacagttttaattgtggaggagaatttttctactgtaattcaacacaactgtttaatagtacttggaataataatactgaagggtcaaataacactgaaggaaatactatcacactcccatgcagaataaaacaaattataaacatgtggcaggaagtaggaaaagcaatgtatgcccctcccatcagaggacaaattagatgttcatcaaatattacagggctgctattaacaagagatggtggtattaatgagaatgggaccgagatcttcagacctggaggaggagatatgagggacaattggagaagtgaattatataaatataaagtagtaaaaattgaaccattaggagtagcacccaccaaggcaaagagaagagtggtgcaaagagaaaaaagagcagtgggaataggagctgtgttccttgggttcttgggagcagcaggaagcactatgggcgcagcgtcaatgacactgacggtacaggccagactattattgtctggtatagtgcaacagcagaacaatttgctgagggctattgaggcgcaacagcgtatgttgcaactcacagtctggggcatcaagcagctccaggcaagagtcctggctgtggaaagatacctaggggatcaacagctcctggggatttggggttgctctggaaaactcatttgcaccactgctgtgccttggaatgctagttggagtaataaatctctggataggatttggaataacatgacctggatggagtgggaaagagaaattgacaattacacaagcgaaatatacaccctaattgaagaatcgcagaaccaacaagaaaagaatgaacaagaattattggaattagataaatgggcaagtttgtggaattggtttgacataacaaaatggctgtggtatataaaaatattcataatgatagtaggaggcttagtaggtttaagactagtttttactgtactttctatagtgaatagagttaggcagggatactcaccattatcgtttcagaccctcctcccagccccgaggggacccgacaggcccgaaggaatcgaagaagaaggtggagagagagacagagacagatccggacgattag

SEQ ID NO: 21 - DNA, 2811 bp, Saccharomyces cerevisiae
attgactcggcagctcatcatgataactccacaattccgttggattttatgcccagggatgctcttcatggatttgattggtctgaagaggatgacatgtcggatggcttgcccttcctgaaaacggaccccaacaataatgggttctttggcgacggttctctcttatgtattcttcgatctattggctttaaaccggaaaattacacgaactctaacgttaacaggctcccgaccatgattacggatagatacacgttggcttctagatccacaacatcccgtttacttcaaagttatctcaataattttcacccctactgccctatcgtgcactcaccgacgctaatgatgttgtataataaccagattgaaatcgcgtcgaaggatcaatggcaaatcctttttaactgcatattagccattggagcctggtgtatagagggggaatctactgatatagatgttttttactatcaaaatgctaaatctcatttgacgagcaaggtcttcgagtcaggttccataattttggtgacagccctacatcttctgtcgcgatatacacagtggaggcagaaaacaaatactagctataattttcacagcttttccataagaatggccatatcattgggcttgaatagggacctcccctcgtccttcagtgatagcagcattctggaacaaagacgccgaatttggtggtctgtctactcttgggagatccaattgtccctgctttatggtcgatccatccagctttctcagaatacaatctccttcccttcttctgtcgacgatgtgcagcgtaccacaacaggtcccaccatatatcatggcatcattgaaacagcaaggctcttacaagttttcacaaaaatctatgaactagacaaaacagtaactgcagaaaaaagtcctatatgtgcaaaaaaatgcttgatgatttgtaatgagattgaggaggtttcgagacaggcaccaaagtttttacaaatggatatttccaccaccgctctaaccaatttgttgaaggaacacccttggctatcctttacaagattcgaactgaagtggaaacagttgtctcttatcatttatgtattaagagattttttcactaattttacccagaaaaagtcacaactagaacaggatcaaaatgatcatcaaagttatgaagttaaacgatgctccatcatgttaagcgatgcagcacaaagaactgttatgtctgtaagtagctatatggacaatcataatgtcaccccatattttgcctggaattgttcttattacttgttcaatgcagtcctagtacccataaagactctactctcaaactcaaaatcgaatgctgagaataacgagaccgcacaattattacaacaaattaacactgttctgatgctattaaaaaaactggccacttttaaaatccagacttgtgaaaaatacattcaagtactggaagaggtatgtgcgccgtttctgttatcacagtgtgcaatcccattaccgcatatcagttataacaatagtaatggtagcgccattaaaaatattgtcggttctgcaactatcgcccaataccctactcttccggaggaaaatgtcaacaatatcagtgttaaatatgtttctcctggctcagtagggccttcacctgtgccattgaaatcaggagcaagtttcagtgatctagtcaagctgttatctaaccgtccaccctctcgtaactctccagtgacaataccaagaagcacaccttcgcatcgctcagtcacgccttttctagggcaacagcaacagctgcaatcattagtgccactgaccccgtctgctttgtttggtggcgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtaaaatgaatcgtagatactgaaaaaccccgcaagttcacttcaactgtgcatcgtgcaccatctcaatttctttcatttatacatcgttttgccttcttttatgtaactatactcctctaagtttcaatcttggccatgtaacctctgatctatagaattttttaaatgactagaattaatgcccatcttttttttggacctaaattcttcatgaaaatatattacgagggcttattcagaagcttcgctcatataacgaaaaaaaagggtttggatcgaacgtaattgagattgattagttaatactcaaaataaaacagctcctaccaccagtgtaaagtagaacgttaatagagcaatgtcttcagacaaatctattgagaaaaatacagatacgatcgcctctgaagttcacgaaggtgataatcattcgaataatttgggttcaatggaggaagagataaaatcaacgccatcagaccaatatgaagagatagctataattccaactgagcccctccattcggacaaagaactaaatgacaagcaacaaagtttaggccatgaagcacccacaaatgtatcaagagaagaacctattgggatc

SEQ ID NO: 22 - DNA, 6 bp, Artificial
  Concensus nuclear receptor DNA binding motif
rgbnnm

SEQ ID NO: 23 - DNA, 6 bp, Artificial
  core half site sequence
aggtca

SEQ ID NO: 24 - DNA, 1389 bp, Homo sapiens
atggacaccaaacatttcctgccgctcgatttctccacccaggtgaactcctccctcacctccccgacggggcgaggctccatggctgccccctcgctgcacccgtccctggggcctggcatcggctccccgggacagctgcattctcccatcagcaccctgagctcccccatcaacggcatgggcccgcctttctcggtcatcagctcccccatgggcccccactccatgtcggtgcccaccacacccaccctgggcttcagcactggcagcccccagctcagctcacctatgaaccccgtcagcagcagcgaggacatcaagccccccctgggcctcaatggcgtcctcaaggtccccgcccacccctcaggaaacatggcttccttcaccaagcacatctgcgccatctgcggggaccgctcctcaggcaagcactatggagtgtacagctgcgaggggtgcaagggcttcttcaagcggacggtgcgcaaggacctgacctacacctgccgcgacaacaaggactgcctgattgacaagcggcagcggaaccggtgccagtactgccgctaccagaagtgcctggccatgggcatgaagcgggaagccgtgcaggaggagcggcagcgtggcaaggaccggaacgagaatgaggtggagtcgaccagcagcgccaacgaggacatgccggtggagaggatcctggaggctgagctggccgtggagcccaagaccgagacctacgtggaggcaaacatggggctgaaccccagctcgccgaacgaccctgtcaccaacatttgccaagcagccgacaaacagcttttcaccctggtggagtgggccaagcggatcccacacttctcagagctgcccctggacgaccaggtcatcctgctgcgggcaggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttag

SEQ ID NO: 25 - DNA, 741 bp, Homo sapiens
atgatttccatcacttctgtgacattctgcttcccaataagtcttcctgtgacttccctatttcccccatcccagattaactcaacagtgtcactccctgggggtgggtctggcccccctgaagatgtgaagccaccagtcttaggggtccggggcctgcactgtccaccccctccaggtggccctggggctggcaaacggctatgtgcaatctgcggggacagaagctcaggcaaacactacggggtttacagctgtgagggttgcaagggcttcttcaaacgcaccatccgcaaagaccttacatactcttgccgggacaacaaagactgcacagtggacaagcgccagcggaaccgctgtcagtactgccgctatcagaagtgcctggccactggcatgaagagggaggcggtacaggaggagcgtcagcggggaaaggacaaggatggggatggggagggggctgggggagcccccgaggagatgcctgtggacaggatcctggaggcagagcttgctgtggaacagaagagtgaccagggcgttgagggtcctgggggaaccgggggtagcggcagcagcgtgagtgttggggtcaatccactctccttcgtgatgggggttgggggaggcagtctaggtctgttctacatcccctccccctcctttcccctcataaccttcctaacactacttgggactggaggtgctgccaaacaaggtctttcaaacatctga

SEQ ID NO: 26 - DNA, 1392 bp, Homo sapiens
atgtatggaaattattctcacttcatgaagtttcccgcaggctatggaggctcccctggccacactggctctacatccatgagcccatcagcagccttgtccacagggaagccaatggacagccaccccagctacacagataccccagtgagtgccccacggactctgagtgcagtggggacccccctcaatgccctgggctctccatatcgagtcatcacctctgccatgggcccaccctcaggagcacttgcagcgcctccaggaatcaacttggttgccccacccagctctcagctaaatgtggtcaacagtgtcagcagttcagaggacatcaagcccttaccagggcttcccgggattggaaacatgaactacccatccaccagccccggatctctggttaaacacatctgtgccatctgtggagacagatcctcaggaaagcactacggggtatacagttgtgaaggctgcaaagggttcttcaagaggacgataaggaaggacctcatctacacgtgtcgggataataaagactgcctcattgacaagcgtcagcgcaaccgctgccagtactgtcgctatcagaagtgccttgtcatgggcatgaagagggaagctgtgcaagaagaaagacagaggagccgagagcgagctgagagtgaggcagaatgtgctaccagtggtcatgaagacatgcctgtggagaggattctagaagctgaacttgctgttgaaccaaagacagaatcctatggtgacatgaatatggagaactcgacaaatgaccctgttaccaacatatgtcatgctgctgacaagcagcttttcaccctcgttgaatgggccaagcgtattccccacttctctgacctcaccttggaggaccaggtcattttgcttcgggcagggtggaatgaattgctgattgcctctttctcccaccgctcagtttccgtgcaggatggcatccttctggccacgggtttacatgtccaccggagcagtgcccacagtgctggggtcggctccatctttgacagagtcctaactgagctggtttccaaaatgaaagacatgcagatggacaagtcggaactgggatgcctgcgagccattgtactctttaacccagatgccaagggcctgtccaacccctctgaggtggagactctgcgagagaaggtttatgccacccttgaggcctacaccaagcagaagtatccggaacagccaggcaggtttgccaagctgctgctgcgcctcccagctctgcgttccattggcttgaaatgcctggagcacctcttcttcttcaagctcatcggggacacccccattgacaccttcctcatggagatgttggagaccccgctgcagatcacctga

SEQ ID NO: 27 - DNA, 16 bp, Artificial
  Concensus LXR response element
ggtttannnnagttca

SEQ ID NO: 28 - DNA, 17 bp, Artificial
  Concensus GAL4 Response element sequence
cggrnnrcynyncnccg

SEQ ID NO: 29 - DNA, 14 bp, Artificial
  Concensus LEXA response element
cgaacnnnngttcg

SEQ ID NO: 30 - DNA, 1221 bp, Homo sapiens
atggcgcttgacggaccagagcagatggagctggaggaggggaaggcaggcagcggactccgccaatattatctgtccaagattgaagaactccagctgattgtgaatgataagagccaaaacctccggaggctgcaggcacagaggaacgaactaaatgctaaagttcgcctattgcgggaggagctacagctgctgcaggagcagggctcctatgtgggggaagtagtccgggccatggataagaagaaagtgttggtcaaggtacatcctgaaggtaaatttgttgtagacgtggacaaaaacattgacatcaatgatgtgacacccaattgccgggtggctctaaggaatgacagctacactctgcacaagatcctgcccaacaaggtagacccattagtgtcactgatgatggtggagaaagtaccagattcaacttatgagatgattggtggactggacaaacagatcaaggagatcaaagaagtgatcgagctgcctgttaagcatcctgagctcttcgaagcactgggcattgctcagcccaagggagtgctgctgtatggacctccaggcactgggaagacactgttggcccgggctgtggctcatcatacggactgtacctttattcgtgtctctggctctgaattggtacagaaattcataggggaaggggcaagaatggtgagggagctgtttgtcatggcacgggaacatgctccatctatcatcttcatggacgaaatcgactccatcggctcctcgcggctggaggggggttctggagggagcagtgaagtgcagcgccagatgctggagttgctcaaccagctcgacggctttgaggccaccaagaacatcaaggttatcatggctactaataggattgatatgctggactcggcactgcttcgcccagggcgcattgacagaaaaattgaattcccaccccccaatgaggaggcccggctggacattttgaagattcattctcggaagatgaacctgacccgggggatcaacctgagaaaaattgctgagctcatgccaggagcatcaggggctgaagtgaagggcgtgtgcacggaagctggcatgtatgccctgcgagaacggcgagtccatgtcactcaggaggactttgagatggcagtagccaaggtcatgcagaaggacagtgagaaaaacatgtccatcaagaaattatggaagtga

SEQ ID NO: 31 - DNA, 3477 bp, Homo sapiens
atgactcatggagaagagcttggctctgatgtgcaccaggattctattgttttaacttacctagaaggattactaatgcatcaggcagcagggggatcaggtactgccgttgacaaaaagtctgctgggcataatgaagaggatcagaactttaacatttctggcagtgcatttcccacctgtcaaagtaatggtccagttctcaatacacatacatatcagggatctggcatgctgcacctcaaaaaagccagactgttgcagtcttctgaggactggaatgcagcaaagcggaagaggctgtctgattctatcatgaatttaaacgtaaagaaggaagctttgctagctggcatggttgacagtgtccgtaaaggcaaacaggatagcacattactggcctctttgcttcagtcattcagctctaggctgcagactgttgctctgtcacaacaaatcaggcagagcctcaaggagcaaggatatgccctcagtcatgattctttaaaagtggagaaggatttaaggtgctatggtgttgcatcaagtcacttaaaaactttgttgaagaaaagtaaagttaaagatcaaaagcctgatacgaatcttcctgatgtgactaaaaacctcatcagagataggtttgcagagtctcctcatcatgttggacaaagtggaacaaaggtcatgagtgaaccgttgtcatgtgctgcaagattacaggctgttgcaagcatggtggaaaaaagggctagtcctgccacctcacctaaacctagtgttgcttgtagccagttagcattacttctgtcaagcgaagcccatttgcagcagtattctcgagaacacgctttaaaaacgcaaaatgcaaatcaagcagcaagtgaaagacttgctgctatggccagattgcaagaaaatggccagaaggatgttggcagttaccagctcccaaaaggaatgtcaagccatcttaatggtcaggcaagaacatcatcaagcaaactgatggctagcaaaagtagtgctacagtgtttcaaaatccaatgggtatcattccttcttcccctaaaaatgcaggttataagaactcactggaaagaaacaatataaaacaagctgctaacaatagtttgcttttacatcttcttaaaagccagactatacctaagccaatgaatggacacagtcacagtgagagaggaagcatttttgaggaaagtagtacacctacaactattgatgaatattcagataacaatcctagttttacagatgacagcagtggtgatgaaagttcttattccaactgtgttcccatagacttgtcttgcaaacacggaactgaaaaatcagaatctgaccaacctgtttccctggataacttcactcaatccttgctaaacacttgggatccaaaagtcccagatgtagatatcaaagaagatcaagatacctcaaagaattctaagctaaactcacaccagaaagtaacacttcttcaattgctacttggccataagaatgaagaaaatgtagaaaaaaacaccagccctcagggagtacacaatgatgtgagcaagttcaatacacaaaattatgcaaggacttctgtgatagaaagccccagtacaaatcggactactccagtgagcactccacctttacttacatcaagcaaagcagggtctcccatcaatctctctcaacactctctggtcatcaaatggaattccccaccatatgtctgcagtactcagtctgaaaagctaacaaatactgcatctaaccactcaatggaccttacaaaaagcaaagacccaccaggagagaaaccagcccaaaatgaaggtgcacagaactctgcaacgtttagtgccagtaagctgttacaaaatttagcacaatgtggaatgcagtcatccatgtcagtggaagagcagagacccagcaaacagctgttaactggaaacacagataaaccgataggtatgattgatagattaaatagccctttgctctcaaataaaacaaatgcagttgaagaaaataaagcatttagtagtcaaccaacaggtcctgaaccagggctttctggttctgaaatagaaaatctgcttgaaagacgtactgtcctccagttgctcctggggaacccaacaaagggaagagtgaaaaaaaaagagaaaactcccttaagagatgaaagtactcaggaacactcagagagagctttaagtgaacaaatactgatggtgaaaataaaatctgagccttgtgatgacttacaaattcctaacacaaatgtgcacttgagccatgatgctaagagtgccccattcttgggtatggctcctgctgtgcagagaagcgcacctgccttaccagtgtccgaagactttaaatcggagcctgtttcacctcaggatttttctttctccaagaatggtctgctaagtcgattgctaagacaaaatcaagatagttacctggcagatgattcagacaggagtcacagaaataatgaaatggcacttctagaatcaaagaatctttgcatggtccctaagaaaaggaagctttatactgagccattagaaaatccatttaaaaagatgaaaaacaacattgttgatgctgcaaacaatcacagtgccccagaagtactgtatgggtccttgcttaaccaggaagagctgaaatttagcagaaatgatcttgaatttaaatatcctgctggtcatggctcagccagcgaaagtgaacacaggagttgggccagagagagcaaaagctttaatgttctgaaacagctgcttctctcagaaaactgtgtgcgagatttgtccccgcacagaagtaactctgtggctgacagtaaaaagaaaggacacaaaaataatgtgaccaacagcaaacctgaatttagcatttcttctttaaatggactgatgtacagttccactcagcccagcagttgcatggataacaggacattttcatacccaggtgtagtaaaaactcctgtgagtcctactttccctgagcacttgggctgtgcagggtctagaccagaatctgggcttttgaatgggtgttccatgcccagtgagaaaggacccattaagtgggttatcactgatgcggagaagaatgagtatgaaaaagactctccaagattgaccaaaaccaacccaatactatattacatgcttcaaaaaggaggcaattctgttgccagtcgagaaacacaagacaaggacatttggagggaggcttcatctgctgaaagtgtctcacaggtcacagccaaagaagagttacttcctactgcagaaacgaaagcttctttctttaatttaagaagcccttacaatagccatatgggaaataatgcttctcgcccacacagcgcaaatggagaagtttatggacttctgggaagcgtgctaacgataaagaaagaatcagaataa

SEQ ID NO: 32 - DNA, 2829 bp, Homo sapiens
atggataccaaggaagagaagaaggaacggaaacaaagttattttgctcgactgaaaaagaaaaaacaagccaaacaaaatgcagagacagcctcagctgtagctacaaggactcatactgggaaggaagataataatacagtagttttagagccagacaagtgcaacattgctgtggaagaggaatatatgactgatgagaaaaaaaagagaaaaagtaatcagttaaaggagatcaggcgtacagaactaaagagatattatagtattgatgacaatcaaaacaaaacacatgataaaaaagagaagaagatggtggttcagaagccccatgggactatggaatacactgctggaaaccaggacaccctaaactccatagcactgaaatttaacatcactcccaataaattggtggaactgaataaacttttcacacatactattgttccaggccaggtcctttttgtgccagatgccaactctccttccagtaccttaaggctatcatcatccagtcctggtgctgctgtctctccttcatcatcagatgcagaatatgataaattgcctgatgctgacttagcacgaaaggccttgaaacccattgaaagagtcttatcgtctacttctgaagaagatgagccaggtgtggtgaaatttttaaaaatgaattgtcgatacttcaccgatggaaagggtgtggttggcggtgttatgatagtgactcctaacaacatcatgtgtgaccctcataaatctgatcctctggttattgaaaatgggtgtgaggagtatggtctcatctgccccatggaagaggttgtttccactgcgctctacaatgacatttctcacatgaagatcaaagatgccttgccatctgacctacctcaggatctttgtcctctgtacaggcctggagaatgggaagacctggcttcagaaaaggatatcaacccattcagtaagttcaaatctatcaacaaggaaaaacgacagcagaatggagagaaaattatgacttcggattccagaccaatagtacctttggagaagtccacaggacatacacctacaaagccctcaggcagctctgtgtcagagaaattaaagaaactggactcctctagggagacatcccatggttctcccacagtgactaagctcagcaaggaaccttccgacacttctgctgcatttgaatctacagccaaagaaaactttctaggggaagatgatgattttgttgacttggaagaactttcttctcaaactggtggtggaatgcacaaaaaagacaccttgaaggagtgcctttctcttgacccagaggaacgaaagaaagctgagtcacaaataaacaattctgccgtggaaatgcaggtgcagtcagccctagcctttttgggaacagagaatgatgttgaactgaagggggcgctagatttagaaacctgtgagaagcaagatataatgccagaagtggacaagcagtctggttcgccagaaagccgagtagaaaacacactgaacatacatgaagatttagataaagttaaactcattgaatattacctgactaagaacaaagaagggccacaggtatctgaaaatttgcagaaaacagaattaagtgatggaaaaagtattgaaccagggggaatagacattacccttagtagttctctttcccaggcgggtgatcccataactgagggcaataaagagccagataagacctgggtgaaaaagggagagcccctcccggtaaaactgaactcttctacagaagcaaatgtgattaaagaggctctagactcctctttggaatctactctggacaacagctgtcaaggtgcacaaatggataataaatctgaagttcagttgtggctgttaaagagaattcaggtacccattgaagatatacttccttcaaaagaagaaaaaagcaagaccccacccatgttcctgtgcatcaaagtgggaaaaccaatgagaaaatcctttgccactcacactgcagccatggtccagcagtacggcaaacggagaaagcagccagagtactggtttgctgttcctcgggagagggtggatcatttgtacacattctttgttcagtggtctcccgatgtctatggaaaagatgccaaagagcaaggctttgtggtggtggagaaggaagaactgaacatgattgacaacttcttcagtgagccaacaaccaagagctgggagatcatcactgttgaagaggcaaagcgcaggaagagcacatgcagctactatgaagacgaggacgaagaggtgctgcctgtcctacggccccacagcgcgctcctggagaatatgcacatcgagcagctggcccgacgccttcctgcaagggtgcaagggtatccatggagactggcctatagcacgttagagcacgggaccagcttaaagacgctctaccggaaatcggcatcactagacagtcctgtcctattggtcatcaaagatatggataatcagatttttggagcatatgcaactcatcctttcaagttcagtgaccactattatggcacaggcgaaacttttctctacacattcagccctcattttaaggtctttaagtggagtggagaaaattcatactttatcaatggagacataagttctttagaacttggtggtggagggggacgatttggtttatggctagatgctgatttataccacggacgaagcaactcttgcagcactttcaataatgatattctttccaaaaaggaagacttcatagttcaggatctggaggtgtgggcatttgattga

SEQ ID NO: 33 - DNA, 555 bp, Homo sapiens
atggccgaccacctgatgctcgccgagggctaccgcctggtgcagaggccgccgtccgccgcggccgcccatggccctcatgcgctccggactctgccgccgtacgcgggcccgggcctggacagtgggctgaggccgcggggggctccgctggggccgccgccgccccgccaacccggggccctggcgtacggggccttcgggccgccgtcctccttccagccctttccggccgtgcctccgccggccgcgggcatcgcgcacctgcagcctgtggcgacgccgtaccccggccgcgcggccgcgccccccaacgctccgggaggccccccgggcccgcagccggcgccaagcgccgcagccccgccgccgcccgcgcacgccctgggcggcatggacgccgaactcatcgacgaggaggcgctgacgtcgctggagctggagctcgggctgcaccgcgtgcgcgagctgcccgagctcttcctgggccagagcgagttcgactgcttctcggacttggggtccgcgccgcccgccggctccgtgagctgctga

SEQ ID NO: 34 - DNA, 3570 bp, Homo sapiens
atggagagtacgcccagcttcctgaagggcaccccaacctgggagaagacggccccagagaacggcatcgtgagacaggagcccggcagcccgcctcgagatggactgcaccatgggccgctgtgcctgggagagcctgctcccttttggaggggcgtcctgagcaccccagactcctggcttccccctggcttcccccagggccccaaggacatgctcccacttgtggagggcgagggcccccagaatggggagaggaaggtcaactggctgggcagcaaagagggactgcgctggaaggaggccatgcttacccatccgctggcattctgcgggccagcgtgcccacctcgctgtggccccctgatgcctgagcatagtggtggccatctcaagagtgaccctgtggccttccggccctggcactgccctttccttctggagaccaagatcctggagcgagctcccttctgggtgcccacctgcttgccaccctacctagtgtctggcctgcccccagagcatccatgtgactggcccctgaccccgcacccctgggtatactccgggggccagcccaaagtgccctctgccttcagcttaggcagcaagggcttttactacaaggatccgagcattcccaggttggcaaaggagcccttggcagctgcggaacctgggttgtttggcttaaactctggtgggcacctgcagagagccggggaggccgaacgcccttcactgcaccagagggatggagagatgggagctggccggcagcagaatccttgcccgctcttcctggggcagccagacactgtgccctggacctcctggcccgcttgtcccccaggccttgttcatactcttggcaacgtctgggctgggccaggcgatgggaaccttgggtaccagctggggccaccagcaacaccaaggtgcccctctcctgagccgcctgtcacccagcggggctgctgttcatcctacccacccactaaaggtgggggtcttggcccttgtgggaagtgccaggagggcctggaggggggtgccagtggagccagcgaacccagcgaggaagtgaacaaggcctctggccccagggcctgtccccccagccaccacaccaagctgaagaagacatggctcacacggcactcggagcagtttgaatgtccacgcggctgccctgaggtcgaggagaggccggttgctcggctccgggccctcaaaagggcaggcagccccgaggtccagggagcaatgggcagtccagcccccaagcggccaccggacccttttccaggcactgcagaacagggggctgggggttggcaggaggtgcgggacacatcgatagggaacaaggatgtggactcgggacagcatgatgagcagaaaggaccccaagatggccaggccagtctccaggacccgggacttcaggacataccatgcctggctctccctgcaaaactggctcaatgccaaagttgtgcccaggcagctggagagggaggagggcacgcctgccactctcagcaagtgcggagatcgcctctgggaggggagctgcagcaggaggaagacacagccaccaactccagctctgaggaaggcccagggtccggccctgacagccggctcagcacaggcctcgccaagcacctgctcagtggtttgggggaccgactgtgccgcctgctgcggagggagcgggaggccctggcttgggcccagcgggaaggccaagggccagccgtgacagaggacagcccaggcattccacgctgctgcagccgttgccaccatggactcttcaacacccactggcgatgtccccgctgcagccaccggctgtgtgtggcctgtggtcgtgtggcaggcactgggcgggccagggagaaagcaggctttcaggagcagtccgcggaggagtgcacgcaggaggccgggcacgctgcctgttccctgatgctgacccagtttgtctccagccaggctttggcagagctgagcactgcaatgcaccaggtctgggtcaagtttgatatccgggggcactgcccctgccaagctgatgcccgggtatgggcccccggggatgcaggccagcagaaggaatcaacacagaaaacgcccccaactccacaaccttcctgcaatggcgacacccacaggaccaagagcatcaaagaggagacccccgattccgctgagaccccagcagaggaccgtgctggccgagggcccctgccttgtccttctctctgcgaactgctggcttctaccgcggtcaaactctgcttgggccatgagcgaatacacatggccttcgcccccgtcactccggccctgcccagtgatgaccgcatcaccaacatcctggacagcattatcgcacaggtggtggaacggaagatccaggagaaagccctggggccggggcttcgagctggcccgggtctgcgcaagggcctgggcctgcccctctctccagtgcggccccggctgcctcccccaggggctttgctgtggctgcaggagccccagccttgccctcggcgtggcttccacctcttccaggagcactggaggcagggccagcctgtgttggtgtcagggatccaaaggacattgcagggcaacctgtgggggacagaagctcttggggcacttggaggccaggtgcaggcgctgagccccctcggacctccccagcccagcagcctgggcagcacaacattctgggagggcttctcctggcctgagcttcgcccaaagtcagacgagggctctgtcctcctgctgcaccgagctttgggggatgaggacaccagcagggtggagaacctagctgccagtctgccacttccggagtactgcgccctccatggaaaactcaacctggcttcctacctcccaccgggccttgccctgcgtccactggagccccagctctgggcagcctatggtgtgagcccgcaccggggacacctggggaccaagaacctctgtgtggaggtggccgacctggtcagcatcctggtgcatgccgacacaccactgcctgcctggcaccgggcacagaaagacttcctttcaggcctggacggggaggggctctggtctccgggcagccaggtcagcactgtgtggcacgtgttccgggcacaggacgcccagcgcatccgccgctttctccagatggtgtgcccggccggggcaggcgccctggagcctggcgccccaggcagctgctacctggatgcagggctgcggcggcgcctgcgggaggagtggggcgtgagctgctggaccctgctccaggcccccggagaggccgtgctggtgcctgcaggggctccccaccaggtgcagggcctggtgagcacagtcagcgtcactcagcacttcctctcccctgagacctctgccctctctgctcagctctgccaccagggacccagccttccccctgactgccacctgctttatgcccagatggactgggctgtgttccaagcagtgaaggtggccgtggggacattacaggaggccaaatag

SEQ ID NO: 35 - DNA, 4263 bp, Homo sapiens
atgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagttaccagtgatgggatattttccccaacagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactataggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcaacagcagcagcagatgcttcaaatgaggcctggtgaaatccccatgggaatgggggctaatccctatggccaagcagcagcatctaaccaactgggttcctggcccgatggcatgttgtccatggaacaagtttctcatggcactcaaaataggcctcttcttaggaattccctggatgatcttgttgggccaccttccaacctggaaggccagagtgacgaaagagcattattggaccagctgcacactcttctcagcaacacagatgccacaggcctggaagaaattgacagagctttgggcattcctgaacttgtcaatcagggacaggcattagagcccaaacaggatgctttccaaggccaagaagcagcagtaatgatggatcagaaggcaggattatatggacagacatacccagcacaggggcctccaatgcaaggaggctttcatcttcagggacaatcaccatcttttaactctatgatgaatcagatgaaccagcaaggcaattttcctctccaaggaatgcacccacgagccaacatcatgagaccccggacaaacacccccaagcaacttagaatgcagcttcagcagaggctgcagggccagcagtttttgaatcagagccgacaggcacttgaattgaaaatggaaaaccctactgctggtggtgctgcggtgatgaggcctatgatgcagccccagcagggttttcttaatgctcaaatggtcgcccaacgcagcagagagctgctaagtcatcacttccgacaacagagggtggctatgatgatgcagcagcagcaacagcagcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatgggacaacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaaatactgctgacatctctgcaccaggacctcttaa

SEQ ID NO: 36 - DNA, 4719 bp, Homo sapiens
atgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagagaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagagttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctcatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaagaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtga

SEQ ID NO: 37 - DNA, 1332 bp, Homo sapiens
atgtctgacatggaggatgatttcatgtgcgatgatgaggaggactacgacctggaatactctgaagatagtaactccgagccaaatgtggatttggaaaatcagtactataattccaaagcattaaaagaagatgacccaaaagcggcattaagcagtttccaaaaggttttggaacttgaaggtgaaaaaggagaatggggatttaaagcactgaaacaaatgattaagattaacttcaagttgacaaactttccagaaatgatgaatagatataagcagctattgacctatattcggagtgcagtcacaagaaattattctgaaaaatccattaattctattcttgattatatctctacttctaaacagatggatttactgcaggaattctatgaaacaacactggaagctttgaaagatgctaagaatgatagactgtggtttaagacaaacacaaagcttggaaaattatatttagaacgagaggaatatggaaagcttcaaaaaattttacgccagttacatcagtcgtgccagactgatgatggagaagatgatctgaaaaaaggtacacagttattagaaatatatgctttggaaattcaaatgtacacagcacagaaaaataacaaaaaacttaaagcactctatgaacagtcacttcacatcaagtctgccatccctcatccactgattatgggagttatcagagaatgtggtggtaaaatgcacttgagggaaggtgaatttgaaaaggcacacactgatttttttgaagccttcaagaattatgatgaatctggaagtccaagacgaaccacttgcttaaaatatttggtcttagcaaatatgcttatgaaatcgggaataaatccatttgactcacaggaggccaagccgtacaaaaatgatccagaaattttagcaatgacgaatttagtaagtgcctatcagaataatgacatcactgaatttgaaaagattctaaaaacaaatcacagcaacatcatggatgatcctttcataagagaacacattgaagagcttttgcgaaacatcagaacacaagtgcttataaaattaattaagccttacacaagaatacatattccttttatttctaaggagttaaacatagatgtagctgatgtggagagcttgctggtgcagtgcatattggataacactattcatggccgaattgatcaagtcaaccaactccttgaactggatcatcagaagaggggtggtgcacgatatactgcactagataaatggaccaaccaactaaattctctcaaccaggctgtagtcagtaaactggcttaa

SEQ ID NO: 38 - DNA, 2499 bp, Homo sapiens
atgtccgaggctggcggggccgggccgggcggctgcggggcaggagccggggcaggggccgggcccggggcgctgcccccgcagcctgcggcgcttccgcccgcgcccccgcagggctccccctgcgccgctgccgccgggggctcgggcgcctgcggtccggcgacggcagtggctgcagcgggcacggccgaaggaccgggaggcggtggctcggcccgaatcgccgtgaagaaagcgcaactacgctccgctccgcgggccaagaaactggagaaactcggagtgtactccgcctgcaaggccgaggagtcttgtaaatgtaatggctggaaaaaccctaacccctcacccactccccccagagccgacctgcagcaaataattgtcagtctaacagaatcctgtcggagttgtagccatgccctagctgctcatgtttcccacctggagaatgtgtcagaggaagaaatgaacagactcctgggaatagtattggatgtggaatatctctttacctgtgtccacaaggaagaagatgcagataccaaacaagtttatttctatctatttaagctcttgagaaagtctattttacaaagaggaaaacctgtggttgaaggctctttggaaaagaaacccccatttgaaaaacctagcattgaacagggtgtgaataactttgtgcagtacaaatttagtcacctgccagcaaaagaaaggcaaacaatagttgagttggcaaaaatgttcctaaaccgcatcaactattggcatctggaggcaccatctcaacgaagactgcgatctcccaatgatgatatttctggatacaaagagaactacacaaggtggctgtgttactgcaacgtgccacagttctgcgacagtctacctcggtacgaaaccacacaggtgtttgggagaacattgcttcgctcggtcttcactgttatgaggcgacaactcctggaacaagcaagacaggaaaaagataaactgcctcttgaaaaacgaactctaatcctcactcatttcccaaaatttctgtccatgctagaagaagaagtatatagtcaaaactctcccatctgggatcaggattttctctcagcctcttccagaaccagccagctaggcatccaaacagttatcaatccacctcctgtggctgggacaatttcatacaattcaacctcatcttcccttgagcagccaaacgcagggagcagcagtcctgcctgcaaagcctcttctggacttgaggcaaacccaggagaaaagaggaaaatgactgattctcatgttctggaggaggccaagaaaccccgagttatgggggatattccgatggaattaatcaacgaggttatgtctaccatcacggaccctgcagcaatgcttggaccagagaccaattttctgtcagcacactcggccagggatgaggcggcaaggttggaagagcgcaggggtgtaattgaatttcacgtggttggcaattccctcaaccagaaaccaaacaagaagatcctgatgtggctggttggcctacagaacgttttctcccaccagctgccccgaatgccaaaagaatacatcacacggctcgtctttgacccgaaacacaaaacccttgctttaattaaagatggccgtgttattggtggtatctgtttccgtatgttcccatctcaaggattcacagagattgtcttctgtgctgtaacctcaaatgagcaagtcaagggctatggaacacacctgatgaatcatttgaaagaatatcacataaagcatgacatcctgaacttcctcacatatgcagatgaatatgcaattggatactttaagaaacagggtttctccaaagaaattaaaatacctaaaaccaaatatgttggctatatcaaggattatgaaggagccactttaatgggatgtgagctaaatccacggatcccgtacacagaattttctgtcatcattaaaaagcagaaggagataattaaaaaactgattgaaagaaaacaggcacaaattcgaaaagtttaccctggactttcatgttttaaagatggagttcgacagattcctatagaaagcattcctggaattagagagacaggctggaaaccgagtggaaaagagaaaagtaaagagcccagagaccctgaccagctttacagcacgctcaagagcatcctccagcaggtgaagagccatcaaagcgcttggcccttcatggaacctgtgaagagaacagaagctccaggatattatgaagttataaggttccccatggatctgaaaaccatgagtgaacgcctcaagaataggtactacgtgtctaagaaattattcatggcagacttacagcgagtctttaccaattgcaaagagtacaacgccgctgagagtgaatactacaaatgtgccaatatcctggagaaattcttcttcagtaaaattaaggaagctggattaattgacaagtga

SEQ ID NO: 39 - DNA, 2397 bp, Homo sapiens
atggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacggaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccggtgaactgagggacagtgatttcagtaatgaacaattctccaaactacctatgtttataaattcaggactagccatggatggcctgtttgatgacagcgaagatgaaagtgataaactgagctacccttgggatggcacacaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcaggtaa

SEQ ID NO: 40 - DNA, 3075 bp, Homo sapiens
atggcggggaacgactgcggcgcgctgctggacgaagagctctcctccttcttcctcaactatctcgctgacacgcagggtggagggtccggggaggagcaactctatgctgactttccagaactcgacctctcccagctggatgccagcgactttgactcggccacctgctttggggagctgcagtggtgcccagagaactcagagactgaacccaaccagtacagccccgatgactccgagctcttccagattgacagtgagaatgaggccctcctggcagagctcaccaagaccctggatgacatccctgaagatgacgtgggtctggctgccttcccagccctggatggtggagacgctctatcatgcacctcagcttcgcctgccccctcatctgcaccccccagccctgccccggagaagccctcggccccagcccctgaggtggacgagctctcactgctgcagaagctcctcctggccacatcctacccaacatcaagctctgacacccagaaggaagggaccgcctggcgccaggcaggcctcagatctaaaagtcaacggccttgtgttaaggcggacagcacccaagacaagaaggctcccatgatgcagtctcagagccgaagttgtacagaactacataagcacctcacctcggcacagtgctgcctgcaggatcggggtctgcagccaccatgcctccagagtccccggctccctgccaaggaggacaaggagccgggtgaggactgcccgagcccccagccagctccagcctctccccgggactccctagctctgggcagggcagaccccggtgccccggtttcccaggaagacatgcaggcgatggtgcaactcatacgctacatgcacacctactgcctcccccagaggaagctgcccccacagacccctgagccactccccaaggcctgcagcaacccctcccagcaggtcagatcccggccctggtcccggcaccactccaaagcctcctgggctgagttctccattctgagggaacttctggctcaagacgtgctctgtgatgtcagcaaaccctaccgtctggccacgcctgtttatgcctccctcacacctcggtcaaggcccaggccccccaaagacagtcaggcctcccctggtcgcccgtcctcggtggaggaggtaaggatcgcagcttcacccaagagcaccgggcccagaccaagcctgcgcccactgcggctggaggtgaaaagggaggtccgccggcctgccagactgcagcagcaggaggaggaagacgaggaagaagaggaggaggaagaggaagaagaaaaagaggaggaggaggagtggggcaggaaaaggccaggccgaggcctgccatggacgaagctggggaggaagctggagagctctgtgtgccccgtgcggcgttctcggagactgaaccctgagctgggcccctggctgacatttgcagatgagccgctggtcccctcggagccccaaggtgctctgccctcactgtgcctggctcccaaggcctacgacgtagagcgggagctgggcagccccacggacgaggacagtggccaagaccagcagctcctacggggaccccagatccctgccctggagagcccctgtgagagtgggtgtggggacatggatgaggaccccagctgcccgcagctccctcccagagactctcccaggtgcctcatgctggccttgtcacaaagcgacccaacttttggcaagaagagctttgagcagaccttgacagtggagctctgtggcacagcaggactcaccccacccaccacaccaccgtacaagcccacagaggaggatcccttcaaaccagacatcaagcatagtctaggcaaagaaatagctctcagcctcccctcccctgagggcctctcactcaaggccaccccaggggctgcccacaagctgccaaagaagcacccagagcgaagtgagctcctgtcccacctgcgacatgccacagcccagccagcctcccaggctggccagaagcgtcccttctcctgttcctttggagaccatgactactgccaggtgctccgaccagaaggcgtcctgcaaaggaaggtgctgaggtcctgggagccgtctggggttcaccttgaggactggccccagcagggtgccccttgggctgaggcacaggcccctggcagggaggaagacagaagctgtgatgctggcgccccacccaaggacagcacgctgctgagagaccatgagatccgtgccagcctcaccaaacactttgggctgctggagaccgccctggaggaggaagacctggcctcctgcaagagccctgagtatgacactgtctttgaagacagcagcagcagcagcggcgagagcagcttcctcccagaggaggaagaggaagaaggggaggaggaggaggaggacgatgaagaagaggactcaggggtcagccccacttgctctgaccactgcccctaccagagcccaccaagcaaggccaaccggcagctctgttcccgcagccgctcaagctctggctcttcaccctgccactcctggtcaccagccactcgaaggaacttcagatgtgagagcagagggccgtgttcagacagaacgccaagcatccggcacgccaggaagcggcgggaaaaggccattggggaaggccgcgtggtgtacattcaaaatctctccagcgacatgagctcccgagagctgaagaggcgctttgaagtgtttggtgagattgaggagtgcgaggtgctgacaagaaataggagaggcgagaagtacggcttcatcacctaccggtgttctgagcacgcggccctctctttgacaaagggcgctgccctgaggaagcgcaacgagccctccttccagctgagctacggagggctccggcacttctgctggcccagatacactgactacgattccaattcagaagaggcccttcctgcgtcagggaaaagcaagtatgaagccatggattttgacagcttactgaaagaggcccagcagagcctgcattgataa

SEQ ID NO: 41 - DNA, 1845 bp, Homo sapiens
atgaataccttccaagaccagagtggcagctccagtaatagagaaccccttttgaggtgtagtgatgcacggagggacttggagcttgctattggtggagttctccgggctgaacagcaaattaaagataacttgcgagaggtcaaagctcagattcacagttgcataagccgtcacctggaatgtcttagaagccgtgaggtatggctgtatgaacaggtggaccttatttatcagcttaaagaggagacacttcaacagcaggctcagcagctctactcgttattgggccagttcaattgtcttactcatcaactggagtgtacccaaaacaaagatctagccaatcaagtctctgtgtgcctggagagactgggcagtttgacccttaagcctgaagattcaactgtcctgctctttgaagctgacacaattactctgcgccagaccatcaccacatttgggtctctcaaaaccattcaaattcctgagcacttgatggctcatgctagttcagcaaatattgggcccttcctggagaagagaggctgtatctccatgccagagcagaagtcagcatccggtattgtagctgtccctttcagcgaatggctccttggaagcaaacctgccagtggttatcaagctccttacatacccagcaccgacccccaggactggcttacccaaaagcagaccttggagaacagtcagacttcttccagagcctgcaatttcttcaataatgtcgggggaaacctaaagggcttagaaaactggctcctcaagagtgaaaaatcaagttatcaaaagtgtaacagccattccactactagttctttctccattgaaatggaaaaggttggagatcaagagcttcctgatcaagatgagatggacctatcagattggctagtgactccccaggaatcccataagctgcggaagcctgagaatggcagtcgtgaaaccagtgagaagtttaagctcttattccagtcctataatgtgaatgattggcttgtcaagactgactcctgtaccaactgtcagggaaaccagcccaaaggtgtggagattgaaaacctgggcaatctgaagtgcctgaatgaccacttggaggccaagaaaccattgtccacccccagcatggttacagaggattggcttgtccagaaccatcaggacccatgtaaggtagaggaggtgtgcagagccaatgagccctgcacaagctttgcagagtgtgtgtgtgatgagaattgtgagaaggaggctctgtataagtggcttctgaagaaagaaggaaaggataaaaatgggatgcctgtggaacccaaacctgagcctgagaagcataaagattccctgaatatgtggctctgtcctagaaaagaagtaatagaacaaactaaagcaccaaaggcaatgactccttctagaattgctgattccttccaagtcataaagaacagccccttgtcggagtggcttatcaggcccccatacaaagaaggaagtcccaaggaagtgcctggtactgaagacagagctggcaaacagaagtttaaaagccccatgaatacttcctggtgttcctttaacacagctgactgggtcctgccaggaaagaagatgggcaacctcagccagttatcttctggagaagacaagtggctgcttcgaaagaaggcccaggaagtattacttaattcacctctacaggaggaacataacttccccccagaccattatggcctccctgcagtttgtgatctctttgcctgtatgcagcttaaagttgataaagagaagtggttatatcgaactcctctacagatgtga

SEQ ID NO: 42 - DNA, 426 bp, Mus musculus
atggcaggtgaagaaatgaatgaagattatcccgtagaaattcacgagtctttaacagccctggagagctccctgggtgctgtggatgacatgctgaagaccatgatggctgtttctagaaatgagttgttgcagaagttggacccattggaacaagcaaaggtggatttagtttctgcatacaccttaaattcaatgttttgggtttatttggcaactcaaggagttaatcccaaagagcatccagtgaagcaggaactggaaagaatcagagtctacatgaacagagttaaagaaataacagacaagaagaaggctgccaagctggacagaggtgctgcttcgagatttgtcaagaacgcactctgggaacccaaagcaaaaagcacaccaaaagtggctaataaagggaaaagcaaacactaa

SEQ ID NO: 43 - protein, 19 aa, Artificial
  Synthetic co-repressor peptide
RLITLADHICQIITQDFAR

SEQ ID NO: 44 - protein, 16 aa, Artificial
  Synthetic co-repressor peptide
ASNLGLEDIIRKALMG

SEQ ID NO: 45 - protein, 19 aa, Artificial
  Synthetic co-repressor peptide
RVVTLAQHISEVITQDYTR

SEQ ID NO: 46 - protein, 25 aa, Artificial
  Synthetic co-activator peptide
CPSSHSSLTERHKILHRLLQEGSPS

SEQ ID NO: 47 - protein, 20 aa, Artificial
  Synthetic co-activator peptide
KYSQTSHKLVQLLTTTAEQQ

SEQ ID NO: 48 - protein, 20 aa, Artificial
  Synthetic co-activator peptide
SLTARHKILHRLLQEGSPSD

SEQ ID NO: 49 - protein, 20 aa, Artificial
  Synthetic co-activator peptide
KESKDHQLLRYLLDKDEKDL

SEQ ID NO: 50 - protein, 19 aa, Artificial
  Synthetic co-activator peptide
HDSKGQTLLQLLTTKADQM

SEQ ID NO: 51 - protein, 20 aa, Artificial
  Synthetic co-activator peptide
SLKEKHKILHRLLQDSSSPV

SEQ ID NO: 52 - protein, 20 aa, Artificial
  Synthetic co-activator peptide
PKKKENALLRYLLDKDDTKD

SEQ ID NO: 53 - protein, 20 aa, Artificial
  Synthetic co-activator peptide
LESKGHKKLLQLLTCSSDDR

SEQ ID NO: 54 - protein, 20 aa, Artificial
  Synthetic co-activator peptide
LLQEKHRILHKLLQNGNSPA

SEQ ID NO: 55 - protein, 20 aa, Artificial
  Synthetic co-activator peptide
KKKENNALLRYLLDRDDPSD

SEQ ID NO: 56 - protein, 20 aa, Artificial
  Synthetic co-activator peptide
SKVSQNPILTSLLQITGNGG

SEQ ID NO: 57 - protein, 20 aa, Artificial
  Synthetic co-activator peptide
GNTKNHPMLMNLLKDNPAQD

SEQ ID NO: 58 - protein, 20 aa, Artificial
  Synthetic co-activator peptide
DAASKHKQLSELLRGGSGSS

SEQ ID NO: 59 - protein, 21 aa, Artificial
  Synthetic co-activator peptide
DAASKHKQLLRYLLRGGSGSS

SEQ ID NO: 60 - protein, 20 aa, Artificial
  Synthetic co-activator peptide
DAASKHKQLSELLDKDEKDL

SEQ ID NO: 61 - protein, 20 aa, Artificial
  Synthetic co-activator peptide
DAASKHKLLRYLLDKDEKDL

SEQ ID NO: 62 - protein, 19 aa, Artificial
  Synthetic co-activator peptide
KESKDHQLSELLDKDEKDL

SEQ ID NO: 63 - protein, 20 aa, Artificial
  Synthetic co-activator peptide
KESKDHQLLRYLLRGGSGSS

SEQ ID NO: 64 - protein, 19 aa, Artificial
  Synthetic co-activator peptide
KESKDHQLSELLRGGSGSS

SEQ ID NO: 65 - protein, 20 aa, Artificial
  Synthetic co-activator peptide
KESKKHKQLRYLLRGGSGSS

SEQ ID NO: 66 - protein, 20 aa, Artificial
  Synthetic co-activator peptide
DAASDHQLLRYLLRGGSGSS

SEQ ID NO: 67 - protein, 20 aa, Artificial
  Synthetic co-activator peptide
KESKDHQLLRYLLDKGSGSS

SEQ ID NO: 68 - protein, 20 aa, Artificial
  Synthetic co-activator peptide
KESKDHQLLRYLLRGDEKDL

SEQ ID NO: 69 - protein, 20 aa, Artificial
  Synthetic co-activator peptide
KESKDHQLLRYLLRGGEKDL

SEQ ID NO: 70 - protein, 20 aa, Artificial
  Synthetic co-activator peptide
KESKDHQLLRYLLRKDEKDL

SEQ ID NO: 71 - protein, 20 aa, Artificial
  Synthetic co-activator peptide
DAASKHKLLRYLLRGGSGSS

SEQ ID NO: 72 - protein, 20 aa, Artificial
  Synthetic co-activator peptide
KESKKHQLLRYLLRGGSGSS

SEQ ID NO: 73 - protein, 20 aa, Artificial
  Synthetic co-activator peptide
KESKDHKLLRYLLRGGSGSS

SEQ ID NO: 74 - protein, 20 aa, Artificial
  Synthetic co-activator peptide
KESKDHQQLRYLLRGGSGSS

SEQ ID NO: 75 - protein, 20 aa, Artificial
  Synthetic co-activator peptide
KESKDHQLLSYLLRGGSGSS

SEQ ID NO: 76 - protein, 20 aa, Artificial
  Synthetic co-activator peptide
KESKDHQLLRELLRGGSGSS

SEQ ID NO: 77 - protein, 20 aa, Artificial
  Synthetic co-activator peptide
KESKDHQQLRYLLDKDEKDL

SEQ ID NO: 78 - protein, 20 aa, Artificial
  Synthetic co-activator peptide
DAASKHKLLSELLRGGSGSS

SEQ ID NO: 79 - protein, 21 aa, Artificial
  Synthetic co-activator peptide
DAASKHKLLRYLLDRGGSGSS

SEQ ID NO: 80 - protein, 20 aa, Artificial
  Synthetic co-activator peptide
DAASKHKQLSELLDGGSGSS

SEQ ID NO: 81 - protein, 20 aa, Artificial
  Synthetic co-activator peptide
KESKDHQLLRYLLRKDEKDL

SEQ ID NO: 82 - protein, 17 aa, Artificial
  Synthetic co-activator peptide
GYVNADLNYLLGSASTF

SEQ ID NO: 83 - protein, 17 aa, Artificial
  Synthetic co-activator peptide
GDDDNPLITLLTGAHSY

SEQ ID NO: 84 - protein, 17 aa, Artificial
  Synthetic co-activator peptide
IANNALLYALLSDHGAH

SEQ ID NO: 85 - protein, 17 aa, Artificial
  Synthetic co-repressor peptide
IGCTSALSRLLINYGDL

SEQ ID NO: 86 - protein, 16 aa, Artificial
  Synthetic co-repressor peptide
ASTMGLEAIIRKALMG

(19) United States
(12) Patent Application Publication
(10) Pub. No.: US 2003/0228607 A1
(43) Pub. Date: Dec. 11, 2003

(54) Screening method and modulators having an improved therapeutic profile

(76) Inventors: Brandee Lynn Wagner, San Diego, CA (US); Ira Glenn Schulman, San Diego, CA (US)

Correspondence Address: BKF JURGENSEN, 800 SILVERADO STREET, 2ND FLOOR, LA JOLLA, CA, 92037, US

(21) Appl. No.: 10/414,692

(22) Filed: Apr. 14, 2003

Related U.S. Application Data
(60) Provisional application No. 60/372,650, filed on Apr. 15, 2002.

(51) Int. Cl. C12Q 1/68; G01N 33/53; G01N 33/567; C07K 14/705

(52) U.S. Cl. 435/6; 435/7.2; 530/358

(57) ABSTRACT

This invention relates to methods for identifying agents useful for treatment of diseases and pathological conditions affected by nuclear receptors and there associated co-factors, and agents and compositions having an improved therapeutic profile identified using such screening methods.

### RELATED APPLICATIONS

Benefit of priority under 35 U.S.C. 119(e) is claimed herein to U.S. provisional application No. 60/372,650, filed Apr. 15, 2002, to Brandee Wagner et al., entitled “Screening method and modulators having an improved therapeutic profile.” The disclosure of the above referenced application is incorporated by reference herein in its entirety.

### FIELD OF THE INVENTION

This invention relates to methods for identifying agents useful for treatment of diseases and pathological conditions affected by nuclear receptors and their associated co-factors, and agents and compositions having an improved therapeutic profile identified using such screening methods.

### BACKGROUND OF THE INVENTION

The average American consumes about 450 mg of cholesterol per day and produces an additional 500 to 1000 mg in the liver and other tissues. Although cholesterol is essential to health, excess serum cholesterol has been implicated in atherosclerosis, heart attack and stroke, and is a leading cause of death in the United States, accounting for approximately 600,000 deaths per year. Because the diet of most western societies is rich in fat and animal products containing cholesterol, the ability to be able to ameliorate the negative effects of excess cholesterol intake would be particularly useful.

Accordingly there is a need for the development of therapeutic agents that can act on the proteins involved in lipid metabolism and transport that could decrease the rate of endogenous cholesterol synthesis, decrease cholesterol uptake or increase the rate of cholesterol transport out of the body. One such class of proteins includes the nuclear receptors directly involved in regulating the transcription of proteins involved in lipid metabolism and transport. Such nuclear receptors include the peroxisome proliferator activated receptors (PPAR α (SEQ. ID. No. 1), PPAR β (SEQ. ID. No. 2), PPAR δ (SEQ. ID. No. 3)) the farnesoid receptor (FXR)(SEQ. ID. No. 4), the Pregnane X-Receptor (PXR) (SEQ. ID. No. 5), Constitutive Androstane Receptor (CAR)(SEQ. ID. No. 6) and the liver X receptors (LXRα and LXRβ)(SEQ. ID. Nos. 6 and 7). The various alternate names, and representative GenBank Accession numbers for these receptors are shown below.

TABLE 1

| | Alternative | |
|---|---|---|
| Receptor Name and Subtype | Names | Accession No |
| PPARα (Peroxisome Proli- | PPARα, | NM_005036 |
| ferator Activated Receptor-α) | | (SEQ. ID. No. 1) |
| NR1C1 | | |
| PPARβ (Peroxisome Proliferator | PPAR-β PPAR-δ, | XM_004285 |
| Activated Receptor-β) NR1C2 | NUC1, FAAR | (SEQ. ID. No. 2) |
| PPARγ (Peroxisome Proliferator | PPARγ, | XM_003059 |
| Activated Receptor-γ) NR1C3 | | (SEQ. ID. No. 3) |
| LXR-β, (Liver X receptor-β) | LXRβ, UR, NER-1, | XM_046419 |
| NR1H2 | OR1 | (SEQ. ID. No. 7) |
| LXR-α, (Liver X receptor-α) | LXRA, XR2, | NM_005693 |
| NR1H3 | RLD1 | (SEQ. ID. No. 6) |
| FXR (Farnesyl X receptor) and | FXR, RIP 14, | NM_005123 |
| splice variants thereof) NR1H4 | HRR1 | (SEQ. ID. No. 4) |
| PXR (Pregnane X-Receptor) | PXR.1, PXR.2, | NM_003889, |
| Isoforms | SXR, ONR1, | NM_022002 |
| NR1I2 | xOR6, BXR | AF364606 |
| | | (SEQ. ID. No. 5) |
| CAR α(Constitutive Androstane | CAR1, MB67 | XM_042458 |
| Receptor) NR1I3 | | (SEQ. ID. No. 8) |

These receptors typically bind to hormone response elements as heterodimers with a common partner, the retinoid X receptors (RXRs) (see, e.g., Levin et al., Nature (1992), Vol. 355, pp. 359-361 and Heyman et al., Cell (1992), Vol. 68, pp. 397-406).

A common problem with the development of useful therapeutic agents to treat metabolic disease based on the regulation of such nuclear receptors is the difficulty of developing agents that can selectively produce the desired metabolic outcome without inducing unwanted side effects.

For example the LXR nuclear receptors are potentially important targets for the development of therapeutic agents for the treatment of a range of diseases including coronary heart disease, diabetes and disorders associated with a high dietary intake of fat.

In vivo, high cholesterol levels act on LXR to coordinate an increase in the transcription of genes involved in cholesterol transport out of the cell, the synthesis of enzymes involved in the metabolic conversion of cholesterol to bile acids, and an increase in the expression of genes involved in fatty acid synthesis. Thus LXR agonists would be predicted to be useful therapeutic agents for the treatment of disorders associated with hypercholesterolemia. However, while such agents are effective in inhibiting or reversing atherosclerosis and associated coronary heart disease, the practical development of such therapeutic agents has been hampered by the induction of hypertriglyceridemia and liver steatosis.

In this case, one of the desired positive effects of LXR is to increase the expression of the ATP binding Cassette transporter ABCA1 in macrophages in order to increase reverse cholesterol transport out of the cell. Unlike other peripheral cells, macrophages are unable to regulate LDL cholesterol uptake, and therefore must rely solely on reverse cholesterol transport to decrease cellular cholesterol levels. In the development of atherosclerotic lesions associated with hypercholesterolemia, lesions begin when macrophages in the arterial wall become loaded with excess acetylated or oxidized LDL cholesterol, turning the macrophage into a foam cell. By over expressing ABCA1, intracellular cholesterol levels are reduced in the macrophage cells resulting in a reduction in foam cell formation and consequent decrease in atherosclerotic lesion development.

Conversely, in the liver, activation of LXR results in an unwanted side effect of increased serum and hepatic triglyceride levels. It is thought that LXR regulation of sterol regulatory element-binding protein-1c (SREBP1c) expression is at least partially responsible for the elevation of triglycerides observed with LXR agonist administration because SREBP1c is a transcription factor that regulates a number of genes in the triglyceride synthesis pathway.

Thus, while known LXR agonists can provide benefits in the intestine and macrophages, such benefits are countered by deleterious affects in the liver, rendering current LXR compounds ineffective for the treatment of cholesterol and other lipid abnormalities, as well as the resulting cardiovascular diseases. Accordingly there is a need to identify LXR ligands that can selectively activate gene expression in a cell type specific fashion.

Central to this need is the development of partial agonists that can bind to nuclear receptors and induce only a subset of the physiological responses induced by a full agonist. Thus by selecting compounds that act to modulate the cell type specific, rather than universal actions of nuclear receptors, it is possible to develop ligands that act in a cell type specific fashion. Specifically it is possible to develop LXR specific compounds that activate ABCA1 expression in macrophages and ileum, but fail to activate SREBP1c expression in the liver.

The development of such compounds relies upon the creation of synthetic ligands that cause modified conformational changes in the nuclear receptor to produce a partial, rather than complete set of orchestrated changes in nuclear receptor activity. These altered conformational states result in modified protein-protein interactions—for example those that mediate receptor dimerization, interaction with heat shock proteins, nuclear localization and the interaction of the nuclear receptor with co-activators and co-repressors. By combining an understanding of the structural basis for these changes with appropriate screening methodologies it is possible to develop ligands that act to selectively modulate one or more of these functions.

For example, X-ray crystallographic analysis suggest that ligand dependent changes in the conformation of the ligand binding domain (LBD) result in the formation of a surface that facilitates interactions with cofactors, such as co-activator and co-repressor proteins. In the case of co-activators, binding is mediated by the presence of a conserved alpha helical LXXLL (SEQ. ID. No. 9) motif, while in the case of co-repressors, the interaction domain contains a conserved LXXi/HIXXXI/L (SEQ. ID. No. 10) motif. Both motifs bind to a hydrophobic cleft on the surface of the LBD that is bounded on one side by the AF-2 domain, and on the other side, by the end of helix three within the LBD.

In the presence of an agonist, structural rearrangements occur that result in the movement of a highly conserved glutamate residue in the AF-2 domain close to hydrophobic pocket of the co-factor interaction domain. As a consequence of the conformational change, interactions of the LBD with co-activators are favored while those with repressors are discouraged. Mechanistically it is believed that in the presence of an agonist, a conserved lysine residue in helix three, together with the glutamate from the AF-2 domain, form a charge clamp that grips a helix of the specific length specified by the LXXLL (SEQ. ID. No. 9) motif, thereby stabilizing co-activator interactions and destabilizing co-repressor interactions.

By contrast, when an antagonist binds to the receptor it fails to induce the conformational changes characteristic of an agonist, resulting instead, in co-activator dissociation, rather than recruitment, and enhanced co-repressor binding. Antagonists typically possess the same, or similar, molecular recognition determinants of a full agonist, but in addition contain one or more side chains comprising bulky extensions that prevent or hinder the movement of the AF-2 domain associated with the stablization of co-activator binding.

Partial agonists bind to receptors and induce only part of the changes in the receptor that are induced by agonists. Thus partial agonists can cause partial co-activator recruitment, and partial co-repressor dissociation, or can fail to cause co-activator recruitment, but cause co-repressor dissociation.

Co-activator recruitment and co-repressor dissociation can have cell type specific actions depending on the cellular context of existing transcription factors and repressors. However such effects are typically not observable with traditional screening approaches, such as the co-transfection assay because these assays use tissue cells that typically lack the endogenous complement of competing DNA binding proteins found in vivo, and because the expression plasmids lack the higher order chromatin structure found in living organisms.

The development of such ligands thus requires an understanding of the appropriate assay methodologies to recognize and optimize suitable partial agonists, as well as a precise understanding of the physiological relevance of a particular pattern of co-activator and co-repressor recruitment.

The present invention provides the basis for developing cell type specific nuclear receptor ligands based on the use of multiplexed screening methodologies that enable the selection of compounds that exhibit specific patterns of co-activator and co-repressor recruitment and dissociation. Applicants have discovered for the first time that such specific patterns of co-factor recruitment can provide for novel cell type specific modulators of nuclear receptor activity.

### SUMMARY OF THE INVENTION

The present invention is based in part upon the discovery that gene disruption of the LXR α and β results in an increase in ABCA1 expression in the macrophage and ileum without significantly increasing SREBP1c expression in the liver. The LXR knock out mediated increase in ABCA1 expression in these tissues occurs as a result of the loss of gene repression mediated by the unoccupied LXR receptor and associated co-repressors that normally act to suppress ABCA1 expression in the wild type animal. Accordingly the present applicants have invented new methodology to identify novel partial agonists of LXR that are selected based on their ability to cause the dissociation of co-repressors from the unoccupied LXR without causing substantial co-activator recruitment. Such modulators would be predicted to increase the expression of ABCA1 in the intestine and macrophages without increasing SREBP1c expression in the liver. The differential expression of ABCA1 versus SREBP1c would be predicted to increase HDL levels by increasing reverse cholesterol transport, and by blocking uptake of dietary cholesterol (thereby lowering LDL) without significantly increasing serum and hepatic triglycerides. Therefore, LXR modulators discovered by the methods of the present invention will have an improved therapeutic index for the treatment of atherosclerosis and other lipid abnormalities. Such modulators include partial agonists of LXR that act to reduce co-repressor binding without substantially increasing co-activator binding.

Accordingly in one embodiment, the present invention includes methods to identify compounds that exhibit high affinity binding to the nuclear receptor and act to disrupt, or substantially disrupt the association of the co-repressor with the nuclear receptor complex and prevent, or substantially prevent the recruitment of a co-activator with the nuclear receptor in the presence of an agonist. In this context “prevent the recruitment” means that recruitment of a co-activator is reduced by at least 90% compared to that exhibited in the presence of a full agonist; “substantially prevent the recruitment” means that recruitment of a co-activator is reduced by at least 50% compared to that exhibited in the presence of a full agonist. Such methods provide for the development of compounds that act in a cell type specific fashion to provide for specific actions in a subset of cells, while providing for a different, or no effect in a second subset of cells.

Further such methods are generally applicable to any nuclear receptor where cell type specific effects are required. In particular such methods are especially well suited to nuclear receptors involved in fatty acid, cholesterol, triglyceride and bile acid metabolism such as PPARα (SEQ. ID. No. 1), PPAR β (SEQ. ID. No. 2), PPAR γ (SEQ. ID. No. 3), LXRα (SEQ. ID. No. 6), LXRβ (SEQ. ID. No. 7), FXR (SEQ. ID. No. 4), CAR (SEQ. ID. No. 8) and PXR (SEQ. ID. No.5). In a preferred embodiment such methods are particularly preferred for the group of nuclear receptors selected from the group consisting of LXRα (SEQ. ID. No. 6), LXRβ (SEQ. ID. No. 7), and FXR (SEQ. ID. No. 4). In another aspect, such methods are particularly suited to LXR.

In another aspect, the present invention provides modulators discovered by the methods of the present invention, methods of using such modulators, as well as pharmaceutical compositions containing such modulators that are useful for the noted abnormalities and disease conditions.

In one aspect, the present invention is directed to a multiplexed method for identifying modulators for a nuclear receptor by contacting each of a plurality of test compounds with a nuclear receptor, wherein said nuclear receptor associates in a complex with a heteromeric partner therefore, a co-activator and/or a co-repressor, assaying for the formation or disruption of the complex, and identifying as modulators those test compounds which disrupt the association of the co-repressor with the complex and prevent the association of a co-activator with the complex.

Accordingly in one embodiment the present invention includes a method to identify compounds that bind to a nuclear receptor and exhibit cell type specific actions, said method comprising;

a) contacting a modified host cell with a test compound, wherein said modified host cell comprises:

i) a first fusion protein, comprising a co-activator fused to a first heterologous DNA binding domain,

ii) a second fusion protein comprising a co-repressor fused to a second heterologous DNA binding domain,

iii) a third fusion protein comprising a ligand binding domain of a nuclear receptor fused to a transcription activation domain,

iv) a first reporter gene operably linked to a first transcriptional regulatory sequence specific for said first heterologous DNA binding domain,

v) a second reporter gene operably linked to a second transcriptional regulatory sequence specific for said second heterologous DNA binding domain,

b) identifying those test compounds which cause altered expression of said first reporter gene product and similar, or altered expression of said second reporter gene product compared to a control modified host cell.

In another embodiment of the invention, the invention includes a method to identify compounds that that bind to a nuclear receptor and exhibit cell type specific actions, said method comprising:

a) contacting a first and second modified host cell with a test compound, wherein said first modified host cell comprises:

i) a first fusion protein, comprising a co-activator fused to a first heterologous DNA binding domain,

ii) a second fusion protein comprising a ligand binding domain of a nuclear receptor fused to a first transcription activation domain,

iii) a first reporter gene operably linked to a first transcriptional regulatory sequence specific for said first heterologous DNA binding domain, and

wherein said second modified host cell comprises,

i) a third fusion protein, comprising a co-repressor fused to said first heterologous DNA binding domain or a second heterologous DNA binding domain,

ii) a fourth fusion protein comprising said ligand binding domain of said nuclear receptor fused to said first transcription activation domain or a second transcription activation domain,

iii) a second reporter gene operably linked to said first transcriptional regulatory sequence specific for said first heterologous DNA binding domain or a second transcriptional regulatory sequence specific for said second heterologous DNA binding domain,

b) identifying those test compounds which cause altered expression of said first reporter gene product in said first modified host cell compared to a first modified host control cell, and similar or altered expression of said second reporter gene product in said second modified host cell, compared to a second modified host control cell.

In another embodiment, the present invention includes a method to identify compounds that that bind to a nuclear receptor and exhibit cell type specific actions, said method comprising:

a) contacting a modified host cell with a test compound, wherein said modified host cell comprises:

i) a first fusion protein, comprising a co-activator fused to a first heterologous DNA binding domain,

ii) a second fusion protein comprising a co-repressor fused to a second heterologous DNA binding domain,

iii) a third fusion protein comprising a ligand binding domain of the nuclear receptor of interest fused to a transcription activation domain,

iv) a first reporter gene operably linked to a first transcriptional regulatory sequence specific for said first heterologous DNA binding domain,

v) a relay protein operably linked to a second transcriptional regulatory sequence specific for said second heterologous DNA binding domain,

vi) a second reporter gene operably linked to a third transcriptional regulatory sequence that is repressed by expression of said relay protein,

b) identifying those test compounds which cause altered expression of said first reporter gene product and similar, or altered expression of said second reporter gene product compared to a control modified host cell.

In another embodiment, the invention includes a method to identify compounds that that bind to a nuclear receptor and exhibit cell type specific actions, said method comprising:

a) contacting a first and second modified host cell with a test compound,

wherein said first modified host cell comprises:

i) a first fusion protein, comprising a co-activator fused to a first heterologous DNA binding domain,

ii) a second fusion protein comprising a ligand binding domain of a nuclear receptor fused to a first transcription activation domain,

iii) a first reporter gene operably linked to a first transcriptional regulatory sequence specific for said first heterologous DNA binding domain, and

wherein said second modified host cell comprises:

i) a third fusion protein, comprising a co-repressor fused to said first heterologous binding domain or a second heterologous binding domain,

ii) a fourth fusion protein, comprising said ligand binding domain fused to said first transcription activation domain or a second transcription activation domain,

iii) a relay plasmid comprising DNA encoding a relay protein operably linked to said first transcriptional regulatory sequence specific for said first heterologous DNA binding domain or a second transcriptional regulatory sequence specific for said second heterologous DNA binding domain,

iv) a second reporter gene operably linked to a third transcriptional regulatory sequence that is repressed by expression of said relay protein,

b) identifying those test compounds which cause altered expression of said first reporter gene product in said first modified host cell compared to a first modified host control cell, and similar or altered expression of said second reporter gene product in said second modified host cell, compared to a second modified host control cell.

In one aspect of these methods the control modified host cell is incubated with a known agonist, antagonist or partial agonist. In another aspect the test compounds are incubated with the modified host cells in the presence of a known agonist, partial agonist or antagonist.

In one aspect of these methods, the co-activator comprises the sequence LXXLL (SEQ. ID. No. 9). In another aspect of these methods, the co-activator is derived from, or comprises a sequence substantially identical to, one member of the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39), and PGC-2 (SEQ. ID. No. 40). In another aspect the co-activator is selected from, or comprises a sequence substantially identical to one of, SEQ. ID. Nos. 47 to 85.

In one aspect of these methods, the co-repressor comprises the sequence HIXXXI/L (SEQ. ID. No. 10). In another aspect the co-repressor derived from, or comprises a sequence substantially identical to, one of SEQ. ID. Nos. 43, 44, 45 and 86.

In another aspect, the co-repressor is derived from, or comprises a sequence substantially identical to one of, the group consisting of SMRT (SEQ. ID. No. 14) and N-CoR (SEQ. ID. No. 15).

In another embodiment of the claimed methods, the first heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16) wherein X can be any amino acid, Cys=cysteine, and His=Histidine. In another aspect, the first heterologous DNA binding domain is selected from the group consisting of a nuclear receptor DNA binding domain, a GAL4 DNA binding domain (SEQ. ID. No. 17) and a LexA DNA binding domain (SEQ. ID. No. 18). In another embodiment, the first heterologous DNA binding domain is selected from the group consisting of a GR DNA binding domain, an MR DNA binding domain, an AR DNA binding domain, a PR DNA binding domain and an ER DNA binding domain.

In another embodiment, the second heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16), wherein X can be any amino acid, Cys=cysteine, and His=Histidine. In another aspect, the first heterologous DNA binding domain is selected from the group consisting of a nuclear receptor DNA binding domain, a GAL4 DNA binding domain (SEQ. ID. No. 17) and a LexA DNA binding domain (SEQ. ID. No. 18). In another embodiment, the second heterologous DNA binding domain is selected from the group consisting of a GR DNA binding domain, a MR DNA binding domain, an AR DNA binding domain, a PR DNA binding domain and an ER DNA binding domain.

In another embodiment of the claimed methods, the first and/or second transactivation domain is selected from the group consisting of VP16 (SEQ. ID. No. 19), TAT (SEQ. ID. No. 20), and the GAL4 activation domain (SEQ. ID. No. 21).

In another embodiment, the nuclear receptor of interest is selected from the group consisting of LXRα (SEQ. ID. No. 6), LXRβ (SEQ. ID. No. 7), FXR (SEQ. ID. No. 4), CAR (SEQ. ID. No. 8), PXR (SEQ. ID. No. 5), PPARα (SEQ. ID. No. 1), PPARβ (SEQ. ID. No. 2) and PPARγ (SEQ. ID. No. 3). In a preferred embodiment the nuclear receptor is selected from the group consisting of LXRα (SEQ. ID. No. 6), LXRβ (SEQ. ID. No. 7), and FXR (SEQ. ID. No. 4).

In another embodiment of the methods, the first transcriptional regulatory sequence comprises the sequence -RGBNNM- (SEQ. ID. No. 22),

wherein R is selected from A or G; B is selected from G, C, or T; each N is independently selected from A, T, C or G and M is selected from A or C;

with the proviso that at least 4 nucleotides of said -RGBNNM (SEQ. ID. No. 22) -sequence are identical with the nucleotides at corresponding position of the sequence -AGGTCA- (SEQ. ID. No. 23).

In another embodiment of the methods, the second transcriptional regulatory sequence comprises the sequence -RGBNNM-(SEQ. ID. No. 22),

wherein R is selected from A or G; B is selected from G, C, or T; each N is independently selected from A, T, C or G and M is selected from A or C;

with the proviso that at least 4 nucleotides of said -RGBNNM-(SEQ. ID. No. 22) sequence are identical with the nucleotides at corresponding position of the sequence -AGGTCA- (SEQ. ID. No. 23).

In another aspect of the methods, the first reporter gene is selected from the group consisting of luciferase, a naturally fluorescent protein, β-galactosidase, β-lactamase, alkaline phosphatase and chloramphenicol acetyltransferase.

In another embodiment, the second reporter gene is selected from the group consisting of luciferase, a naturally fluorescent protein, β-galactosidase, β-lactamase, alkaline phosphatase and chloramphenicol acetyltransferase. In one aspect of this embodiment, the naturally fluorescent protein is an enhanced mutant fluorescent protein such as EGFP, YFP, CFP, EBFP or DSred.

In another aspect of the claimed methods, the first reporter gene and the second reporter genes are selected to enable multiplexed analysis. In another aspect, the first and second reporter genes are the same.

In another embodiment of the methods, the test compound has a known Kd for said nuclear receptor of at least 500 nM. In another aspect the test compound has a known Kd for said nuclear receptor of at least 200 nM. In another aspect the test compound has a known Kd for said nuclear receptor of at least 100 nM.

In another aspect, the invention includes a composition comprising,

a) a modified host cell which comprises:

i) a first fusion protein, comprising a co-activator fused to a first heterologous DNA binding domain,

ii) a second fusion protein comprising a co-repressor fused to a second heterologous DNA binding domain,

iii) a third fusion protein comprising a ligand binding domain of the nuclear receptor of interest (“prey”) fused to a transcription activation domain,

iv) a first reporter gene operably linked to a first transcriptional regulatory sequence specific for said first heterologous DNA binding domain,

v) a relay protein operably linked to a second transcriptional regulatory sequence specific for said second heterologous DNA binding domain,

vi) a second reporter gene operably linked to a third transcriptional regulatory sequence that is repressed by expression of said relay protein,

a) a test compound.

In one aspect of these compositions, the co-activator comprises the sequence LXXLL (SEQ. ID. No. 9). In another aspect of these methods, the co-activator is derived from, or comprises a sequence substantially identical to, one member of the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39), and PGC-2 (SEQ. ID. No. 40). In another aspect the co-activator is selected from, or comprises a sequence substantially identical to, one of SEQ. ID. Nos. 47 to 85.

In one aspect of these compositions, the co-repressor comprises the sequence HIXXXI/L (SEQ. ID. No. 10). In another aspect the co-repressor derived from, or comprises a sequence substantially identical to one of, SEQ. ID. Nos. 43, 44, 45 and 86.

In another aspect, the co-repressor is derived from, or comprises a sequence substantially identical to, one of the group consisting of SMRT (SEQ. ID. No. 14) and N-CoR (SEQ. ID. No. 15).

In another embodiment of the claimed compositions, the first heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16) wherein X can be any amino acid, Cys=cysteine, and His=Histidine. In another aspect, the first heterologous DNA binding domain is selected from the group consisting of a nuclear receptor DNA binding domain, a GAL4 DNA binding domain (SEQ. ID. No. 17) and a LexA DNA binding domain (SEQ. ID. No. 18).

In another embodiment, the second heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16), wherein X can be any amino acid, Cys=cysteine, and His=Histidine. In another aspect, the first heterologous DNA binding domain is selected from the group consisting of a nuclear receptor DNA binding domain, a GAL4 DNA binding domain (SEQ. ID. No. 17) and a LexA DNA binding domain (SEQ. ID. No. 18).

In another embodiment of the claimed compositions, the transactivation domain is selected from the group consisting of VP16 (SEQ. ID. No. 19), TAT (SEQ. ID. No. 20), and the GAL4 activation domain (SEQ. ID. No. 21).

In another embodiment, the nuclear receptor of interest is selected from the group consisting of LXRα (SEQ. ID. No. 6), LXRβ (SEQ. ID. No. 7), FXR (SEQ. ID. No. 4), CAR (SEQ. ID. No. 8), PXR (SEQ. ID. No. 5), PPARα (SEQ. ID. No. 1), PPARβ (SEQ. ID. No. 2) and PPARγ (SEQ. ID. No. 3). In a preferred embodiment the nuclear receptor is selected from the group consisting of LXRα (SEQ. ID. No. 6), LXRβ (SEQ. ID. No. 7), and FXR (SEQ. ID. No. 4).

In another embodiment of the compositions the first transcriptional regulatory sequence comprises the sequence -RGBNNM- (SEQ. ID. No. 22),

wherein R is selected from A or G; B is selected from G, C, or T; each N is independently selected from A, T, C or G and M is selected from A or C;

with the proviso that at least 4 nucleotides of said -RGBNNM (SEQ. ID. No. 22) -sequence are identical with the nucleotides at corresponding position of the sequence -AGGTCA- (SEQ. ID. No. 23).

In another embodiment of the compositions the second transcriptional regulatory sequence comprises the sequence -RGBNNM-(SEQ. ID. No. 22),

wherein R is selected from A or G; B is selected from G, C, or T; each N is independently selected from A, T, C or G and M is selected from A or C;

with the proviso that at least 4 nucleotides of said -RGBNNM-(SEQ. ID. No. 22) sequence are identical with the nucleotides at corresponding position of the sequence -AGGTCA- (SEQ. ID. No. 23).

In another embodiment of the compositions the second transcriptional regulatory sequence comprises the sequence -RGBNNM-(SEQ. ID. No. 22),

wherein R is selected from A or G; B is selected from G, C, or T; each N is independently selected from A, T, C or G and M is selected from A or C;

with the proviso that at least 4 nucleotides of said -RGBNNM-(SEQ. ID. No. 22) sequence are identical with the nucleotides at corresponding position of the sequence -AGGTCA- (SEQ. ID. No. 23).

In another aspect of the compositions, the first reporter gene is selected from the group consisting of luciferase, a naturally fluorescent protein, β-galactosidase, β-lactamase, alkaline phosphatase and chloramphenicol acetyltransferase.

In another embodiment, the second reporter gene is selected from the group consisting of luciferase, a naturally fluorescent protein, β-galactosidase, β-lactamase, alkaline phosphatase and chloramphenicol acetyltransferase. In one aspect of this embodiment the naturally fluorescent protein is an enhanced mutant fluorescent protein such as EGFP, YFP, CFP, EBFP or DSred.

In another aspect of the claimed compositions, the first reporter gene and the second reporter genes are selected to enable multiplexed analysis.

In another embodiment of the compositions, the test compound has a known Kd for said nuclear receptor of at least 500 nM. In another aspect, the test compound has a known Kd for said nuclear receptor of at least 200 nM. In another aspect the test compound has a known Kd for said nuclear receptor of at least 100 nM.

In another embodiment of the invention, the invention includes a method to identify compounds that bind to a nuclear receptor and exhibit cell type specific actions, said method comprising:

a) providing a composition comprising,

i) an affinity support, comprising a first fusion protein comprising a ligand binding domain of the nuclear receptor fused to an affinity tag that couples said first fusion protein to said affinity support,

ii) a second fusion protein, comprising a co-activator coupled to a first detectable label,

iii) a third fusion protein comprising a co-repressor coupled to a second detectable label,

b) incubating said composition in an aqueous buffer comprising a test compound,

c) detecting the binding of said co-activator and said co-repressor to said first fusion protein,

c) identifying those test compounds which cause altered binding of said co-repressor and similar or altered binding of said co-activator to said nuclear receptor compared to a control composition.

In one embodiment of this method binding is detected by fluorescence polarization. In another embodiment, by immunochemical detection. In another embodiment, by measurement of binding via quantification of bound co-factor via detection of the first and second detectable labels.

In another embodiment, the present invention includes a method to identify compounds that exhibit cell type specific actions on a nuclear receptor, comprising:

a) providing a composition comprising,

i) a ligand binding domain of a nuclear receptor, and

ii) a co-activator coupled to a first detectable label, and

iii) a co-repressor coupled to a second detectable label,

b) incubating said composition in an aqueous buffer comprising a test compound,

c) detecting the binding of said co-activator and said co-repressor with said ligand binding domain,

d) identifying those test compounds which cause altered binding of said co-repressor and similar or altered binding of said co-activator to said ligand binding domain compared to a control composition.

In one embodiment of this method, binding is detected by measuring changes in the fluorescence polarization of the first and second detectable label.

In another embodiment, the present invention includes a method to identify compounds that bind to a nuclear receptor and exhibit cell type specific actions, said method comprising:

a) providing first and second compositions, wherein said first composition comprises;

i) a ligand binding domain of a nuclear receptor, coupled to a first detectable label, and

ii) a co-activator coupled to a second detectable label, and wherein said second composition comprises;

iii) said ligand binding domain, coupled to said first detectable label, and

iv) a co-repressor coupled to said second detectable label,

b) incubating said first composition and said second composition in an aqueous buffer comprising a test compound,

c) detecting the binding of said co-activator with said ligand binding domain in said first composition and detecting the binding of said co-repressor with said ligand binding domain in said second composition,

d) identifying those test compounds which cause altered binding of said co-repressor and similar or altered binding of said co-activator to said ligand binding domain compared to a control composition.

In one embodiment of this method, binding is detected by fluorescence polarization. In another embodiment, by immunochemical detection. In another embodiment, by measurement of binding via quantification of bound co-factor via detection of the first and second detectable labels. In another embodiment by FRET, time resolved FRET or a SPA assay.

In another aspect, the present invention includes a composition comprising,

i) an affinity support, comprising a first fusion protein comprising a ligand binding domain of the nuclear receptor fused to an affinity tag that couples said first fusion protein to said affinity support,

ii) a second fusion protein, comprising a co-activator coupled to a first detectable label,

iii) a third fusion protein comprising a co-repressor coupled to a second detectable label, wherein said first detectable label and said second detectable label are independently quantifiable,

iv) a test compound.

In one aspect of the claimed compositions and methods, the test compound has a known Kd for said nuclear receptor of at least 500 nM. In another aspect, the test compound has a known Kd for said nuclear receptor of at least 200 nM. In another aspect, the test compound has a known Kd for said nuclear receptor of at least 100 nM.

In another aspect, the first and second detectable labels are selected from the group consisting of a radiolabel, affinity tag, fluorescent or luminescent moiety and an enzymatic moiety.

In one aspect, the affinity tag is selected from the group consisting of biotin, a binding site for an antibody, a metal binding domain, a FLASH binding domain, and a glutathione binding domain.

In another aspect, the radiolabel is selected from the group consisting of ³H, ¹⁴C, ³⁵S, ¹²⁵I, and ¹³¹I.

In another aspect, the enzymatic moiety is selected from the group consisting of horseradish peroxidase, β-galactosidase, β-lactamase, luciferase and alkaline phosphatase.

In another aspect, the fluorescent or luminescent moiety is selected from the group consisting of fluorescein, a naturally fluorescent protein, rhodamine, and a lanthanide.

In one aspect, the luminescent moiety comprises a lanthanide selected from the group consisting of Europium (Eu), Samarium (Sm) and Terbium (Tb).

In one aspect of these methods and compositions, the co-activator comprises the sequence LXXLL (SEQ. ID. No. 9). In another aspect of these methods, the co-activator is derived from, or comprises a sequence substantially identical to, one member of the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39) and PGC-2 (SEQ. ID. No. 40). In another aspect, the co-activator is selected from, or comprises a sequence substantially identical to, one of SEQ. ID. Nos. 47 to 85.

In one aspect of these methods and compositions, the co-repressor comprises the sequence HIXXXI/L (SEQ. ID. No. 10). In another aspect, the co-repressor is derived from, or comprises a sequence substantially identical to, one of SEQ. ID. Nos. 43, 44, 45 and 86.

In another aspect, the co-repressor is derived from, or comprises a sequence substantially identical to, one of the group consisting of SMRT (SEQ. ID. No. 14) and N-CoR (SEQ. ID. No. 15).

In another embodiment, the nuclear receptor of interest is selected from the group consisting of LXRα (SEQ. ID. No. 6), LXRβ (SEQ. ID. No. 7), FXR (SEQ. ID. No. 4), CAR (SEQ. ID. No. 8), PXR (SEQ. ID. No. 5), PPARα (SEQ. ID. No. 1), PPARβ (SEQ. ID. No. 2) and PPARγ (SEQ. ID. No. 3). In a preferred embodiment the nuclear receptor is selected from the group consisting of LXRα (SEQ. ID. No. 6), LXRβ (SEQ. ID. No. 7), and FXR (SEQ. ID. No. 4).

In another embodiment, the invention includes a kit comprising a first fusion protein comprising a ligand binding domain of the nuclear receptor fused to an affinity tag that couples said first fusion protein to an affinity support; a second fusion protein, comprising a co-activator, or a fragment thereof, coupled to a first detectable label; and a third fusion protein comprising a co-repressor or a fragment thereof, coupled to a second detectable label, wherein said first detectable label and said second detectable label are independently quantifiable, and optionally, instructions for use.

In one aspect of the kit, the co-activator comprises the sequence LXXLL (SEQ. ID. No. 9). In another aspect of the kit, the co-activator is derived from, or comprises a sequence substantially identical to, one member of the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39) and PGC-2 (SEQ. ID. No. 40). In another aspect the co-activator is selected from, or comprises a sequence substantially identical to, one of SEQ. ID. Nos. 47 to 85.

In one aspect of the kit, the co-repressor comprises the sequence HIXXXI/L (SEQ. ID. No. 10). In another aspect the co-repressor is derived from, or comprises a sequence substantially identical to, one of SEQ. ID. Nos. 43, 44, 45 and 86.

In another aspect, the co-repressor is derived from, or comprises a sequence substantially identical to, one of the group consisting of SMRT (SEQ. ID. No. 14) and N-CoR (SEQ. ID. No. 15).

### BRIEF DESCRIPTION OF THE DRAWINGS

FIG. 1. Effects of LXR on plasma HDL. Blood was collected from 11 eight-week-old male animals during the middle of a 12-hour light cycle. Plasma HDL levels were measured by an enzymatic assay for total cholesterol (Sigma, St. Louis, Mo.) following precipitation of non-HDL cholesterol by a heparin-manganese precipitating reagent (Wako Diagnostics, Richmond, Va.).

FIG. 2. LXR dependent activation and repression of ABCA1. Thyoglycolate induced peritoneal macrophage were isolated from wild type and LXRαβ knockout mice. The cells were plated in DMEM with 10% FBS and induced with either vehicle or 1 μM of the LXR agonist T0901317 (N-(2,2,2,-trifluoro-ethyl)-N-[4-(2,2,2-trifluoro-1-hydroxy-1-trifluoromethyl-ethyl)-phenyl]-benzenesulfonamide)(X-Ceptor Therapeutics, Inc., San Diego, Calif.) for 18 hrs. FIG. 2A. Following induction mRNA was isolated and RT-PCR was used to quantitate the levels of ABCA1 and the cyclophilin. ABCA1 levels were normalized to cyclophilin levels and the results are presented as fold induction above wild type macrophage treated with vehicle. FIG. 2B. For western blot analysis, whole cells extracts were isolated, run on an SDS-PAGE and probed with an antibody specific for ABCA1.

FIG. 3. LXR mediated repression and activation of cholesterol efflux. Thyoglycolate induced peritoneal macrophage were isolated from wild type and LXRαβ knockout mice. Cell were plated at 1×10⁵ cells/ml DMEM with 1% FBS and labeled with [C14]-cholesterol for 48 hrs. Following labeling the media was replaced with serum free media +/−5 μg/ml ApoA1, +/−T0901317. 24 hours later the media is removed and measured for radioactivity. The cells are lysed in 0.2M sodium hydroxide and the radioactivity is measured. The percent efflux is calculated by dividing the radioactivity of the media by the sum radioactivity of the media and cell lysate. ApoA1 dependent efflux is calculated by subtracting ApoA1 independent efflux from the ApoA1 dependent efflux.

FIG. 4. LXR mediated repression is gene specific. Thyoglycolate induced peritoneal macrophage were isolated from wild type and LXRαβ knockout mice. The cells were plated in DMEM with 10% FBS and induced with either vehicle or 1 μM T0901317 for 18 hrs. Following induction mRNA was isolated and RT-PCR was used to quantitate the levels of FIG. 4A, SREBP1c, FIG. 4B, ApoE and cyclophilin. The levels of SREBP1c and ApoE levels are normalized to cyclophilin levels.

FIG. 5. LXR mediated repression of ABCA1 is tissue specific. To measure the LXR dependent regulation of ABCA1 in different tissues wild type and LXRαβ−/− mice were dosed with 10 mg/kg T0901317 for 7 days by oral gavage. After 7 days the animals were sacrificed and intestinal mucosa, quadriceps, and livers were harvested. Mouse embryonic fibroblasts (MEFs) were isolated from embryos harvested at embryonic day 14. MEFs were plated in DMEM+10% FBS and treated with either vehicle or 1 μM T0901317 for 18 hrs. RNA was isolated and RT-PCR was used to quantitate the levels of ABCA1 and cyclophilin in FIG. 5A, intestinal mucosa, FIG. 5B, liver, FIG. 5C, MEFs and FIG. 5D, quadricep. The levels of ABCA1 mRNA are normalized to cyclophilin mRNA.

FIG. 6. LXR interacts with the co-repressors NcoR and SMRT in the absence of ligand. Two-hybrid analysis was performed by transiently transfecting CV-1 cells with a Gal4-luciferase reporter, Gal4-fusions of the receptor interacting domains of NcoR and SMRT, VP16 fusion of the ligand binding domains of LXRα and LXRβ and a β-gal expression vector. Cells were transfected with FUGENE transfection reagent and incubated with vehicle or 1 μM T0901317 overnight. The cells were then lysed and analyzed for β-gal and luciferase activity. The data is presented as luciferase activity normalized to β-gal activity.

FIG. 7. LXR represses Gal4 basal transcription. One-hybrid analysis of LXR was performed by transiently transfecting CV-1 cells with a Gal4-luciferase reporter, Gal4-fusions of full length LXRα or LXRβ and a β-gal reporter. Cells were transfected with FUGENE transfection reagent and incubated without ligand overnight. The cells were then lysed and analyzed for β-gal and luciferase activity. The data is presented as luciferase activity normalized to β-gal activity.

FIG. 8. LXR mediated repression of Gal4 basal transcription is dependent on expression of the co-repressor NCoR. Mouse embryonic fibroblasts (MEFs) were isolated from wild type and NCoR^{-/-} embryos harvested at embryonic day 14. One-hybrid analysis of LXR was performed by transiently transfecting MEFs with a Gal4-luciferase reporter, Gal4-fusions of full length LXRα or LXRβ and a β-gal reporter. Cells were transfected with FUGENE transfection reagent and incubated without ligand overnight. The cells were then lysed and analyzed for β-gal and luciferase activity. The data is presented as luciferase activity normalized to β-gal activity.

FIG. 9. is a scatter plot of a co-activator recruitment assay showing fluorescence emitted from APC at 665 nm in a FRET based (biochemical) co-factor recruitment assay that detects the binding of the SCR-1 co-activator to FXR. In this assay, recruitment of SCR-1 to both LXRα and LXRβ were simultaneously assayed.

FIG. 10. High throughput FXR cofactor recruitment assay Scatter plot of a co-activator recruitment assay showing fluorescence emitted from APC at 665 nm in a FRET based (biochemical) co-factor recruitment assay that detects the binding of the SCR-1 co-activator to FXR. In this assay, 8 nM GST-FXR-LBD was used in the place of LXRα, and LXRβ, with 5 nM Eu-labeled anti-GST antibody; 16 nM biotin-SRC-1 peptide; 20 nM APC-SA in 1× FRET buffer.

FIG. 11 High throughput mammalian two-hybrid recruitment assay Representative Spotfire visualization of data obtained from a screen of 170,000 compounds using the mammalian two-hybrid assay with LXRβ.

### DETAILED DESCRIPTION OF THE INVENTION

Definitions:

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art.

Generally, the nomenclature used hereafter and the laboratory procedures in cell culture, molecular genetics, and nucleic acid chemistry and hybridization described below are those well known and commonly employed in the art. Standard techniques are used for recombinant nucleic acid methods, polynucleotide synthesis, cell culture, and transgene incorporation (e.g., electroporation, microinjection, lipofection). Generally enzymatic reactions, oligonucleotide synthesis, and purification steps are performed according to the manufacturer's specifications. The techniques and procedures are generally performed according to conventional methods in the art and various general references which are provided throughout this document, as well as: Maniatis et al., Molecular Cloning: A Laboratory Manual (1989), 2nd Ed., Cold Spring Harbor, N.Y.; and Berger and Kimmel, Methods in Enzymology, Volume 152, Guide to Molecular Cloning Techniques (1987), Academic Press, Inc., San Diego, Calif., which are incorporated herein by reference. Oligonucleotides can be synthesized on an Applied Bio Systems oligonucleotide synthesizer according to specifications provided by the manufacturer. The procedures are believed to be well known in the art and are provided for the convenience of the reader. All the information contained therein is incorporated herein by reference.

All sequences referred to herein by GenBank database file designation (e.g., GenBank: Humatct4a) or otherwise obtainable by routine search of a publicly-available sequence database or scientific publications are incorporated herein by reference and are publicly available, such as by reconstruction of the sequence by overlapping oligonucleotides or other means.

As used herein, the term “agonist” refers to an agent which produces activation of a nuclear receptor and provides for a substantial increase in binding of one or more coactivator protein(s) to nuclear receptor, and relieves binding of one or more corepressors to the nuclear receptor.

The term “analog” as used herein refers to polypeptides which are comprised of a segment of at least 25 amino acids that has substantial identity to a portion of an LBD, co-activator, or co-repressor, and which has specific binding to a nuclear receptor, coactivator or co-repressor species. Typically, analog polypeptides comprise a conservative amino acid substitution (or addition or deletion) with respect to the naturally-occurring sequence. Analogs typically are at least 20 amino acids long, preferably at least 50 amino acids long or longer, most usually being as long as full-length naturally-occurring polypeptide.

As used herein, the term “antagonist” refers to an agent which opposes the agonist activity of a known agonist of the nuclear receptor LBD, or enhances or stabilizes binding of a co-repressor protein to a nuclear receptor and inhibits binding of one or more coactivators to the nuclear receptor.

The term “detectable label” refers to any moiety that can be selectively detected in a screening assay. Examples include without limitation, radiolabels, (e.g., ³H, ¹⁴C, ³⁵S, ¹²⁵I, ¹³¹I), affinity tags (e.g. biotin/avidin or streptavidin, binding sites for antibodies, metal binding domains, epitope tags, FLASH binding domains (See U.S. Pat. Nos. 6,451,569; 6,054,271; 6,008,378 and 5,932,474), glutathione or maltose binding domains) fluorescent or luminescent moieties (e.g. fluorescein and derivatives, GFP, rhodamine and derivatives, lanthanides etc.), and enzymatic moieties (e.g. horseradish peroxidase, β-galactosidase, β-lactamase, luciferase, alkaline phosphatase). Such detectable labels can be formed in situ, for example, through use of an unlabeled primary antibody which can be detected by a secondary antibody having an attached detectable label.

The term “DNA binding domain” or “DBD” refers to protein domain capable of binding to a specific DNA sequence, and comprising at least one zinc finger sequence. The term “zinc finger sequence” refers to a sequence conforming to the following pattern: X-X-Cys-X₍₁₋₅₎-Cys-X-X-X-X-X-X-X-X-X-X-X-X-His-X₍₃₋₆₎-[His/Cys] (SEQ. ID. No.16). Where X can be any amino acid, the numbers in brackets indicate the number of residues, and Cys=cysteine, and His=Histidine. The positions marked in bold are those that are important for the stable fold of the zinc finger. The final position can be either His or Cys. Typical zinc finger domains are composed of two short beta strands followed by an alpha helix. The amino terminal part of the helix binds the major groove in DNA binding zinc fingers, while the two conserved cysteines and histidines co-ordinate a zinc ion.

As used herein, the term “disrupt” embraces test compounds that cause substantially complete disassociation (i.e. greater than 90% dissociation of bound co-factor from a receptor. The term “substantially disrupt” embraces test compounds which cause at least 50% dissociation of bound co-factor from a receptor.

The term “fragment” as used herein refers to a polypeptide that has an amino-terminal and/or carboxy-terminal deletion, but where the remaining amino acid sequence is identical to, or exhibits substantial identity to, the corresponding positions in the naturally-occurring sequence, for example derived from a co-activator, co-repressor, or interacting peptide fragment as disclosed herein. Fragments typically are at least 14 amino acids long, preferably at least 20 amino acids long, and comprise at least one interaction domain. Larger sequences, for example with about 50 amino acids, or longer, are also intended to be within the scope of the term and may contain multiple repeats of the core sequence or interaction domain.

As used herein, the term “functionally expressed” refers to a coding sequence which is transcribed, translated, post-translationally modified (if relevant), and positioned in a cell such that the protein provides the desired function. With reference to a reporter cassette, functional expression generally means production of a sufficient amount of the encoded cell surface reporter protein to provide a statistically significant detectable signal to report ligand-induced transcriptional effects of a reporter polynucleotide.

As used herein, the term “LBD” or “ligand-binding domain” refers to the protein domain of a nuclear receptor, such as a steroid superfamily receptor or other suitable nuclear receptor as discussed herein, which binds a physiological ligand and thereupon undergoes a conformational change and/or altered intermolecular interaction with an associated protein so as to confer a detectable activity upon a second, linked functional domain.

As used herein, “linked” means in polynucleotide linkage (i.e., phosphodiester linkage) or polypeptide linkage, depending upon the context of usage. “Unlinked” means not linked to another polynucleotide or polypeptide sequence; hence, two sequences are unlinked if each sequence has a free 5′ terminus and a free 3′ terminus.

As used herein, the term “LXR” refers to Liver X Receptors, which are members of the nuclear receptor super family of transcription factors. There are two known LXR receptors, LXRα (SEQ. ID. No. 6) and LXRβ (SEQ. ID. No.7). See, e.g., U.S. application Ser. No. 08/373,935, filed Jan. 13, 1995; Apfel et al., in Mol. Cell. Biol. 14:7025-7035 (1994).

As used herein, the term “modulator” refers to a wide range of test compounds, including, but not limited to natural, synthetic or semi-synthetic organic molecules, proteins, oligonucleotides and antisense, that directly or indirectly influence the activity of LXR in complex including one or more of a heterodimerizing partner therefore, a co-activator and/or a co-repressor. Preferably, a modulator substantially disrupts the association of a co-repressor with the complex without substantially promoting the association of a co-activator with the complex. Furthermore, the precursor of a modulator (i.e., a compound that can be converted into a modulator) is also considered to be a modulator. Similarly, a compound which converts a precursor into a modulator is also considered to be a modulator.

“Naturally fluorescent protein” refers to proteins capable of forming a highly fluorescent, intrinsic chromophore either through the cyclization and oxidation of internal amino acids within the protein or via the enzymatic addition of a fluorescent co-factor. Typically such chromophores can be spectrally resolved from weakly fluorescent amino acids such as tryptophan and tyrosine. Endogenously fluorescent proteins have been isolated and cloned from a number of marine species including the sea pansies Renilla reniformis, R. kollikeri and R. mullerei and from the sea pens Ptilosarcus, Stylatula and Acanthoptilum, as well as from the Pacific Northwest jellyfish, Aequorea Victoria; Szent-Gyorgyi et al. (SPIE conference 1999), D. C. Prasher et al., Gene, 111:229-233 (1992) and red and yellow fluorescent proteins from coral. A variety of mutants of the GFP from Aequorea Victoria have been created that have distinct spectral properties, improved brightness and enhanced expression and folding in mammalian cells compared to the native GFP, (Green Fluorescent Proteins, Chapter 2, pages 19 to 47, edited Sullivan and Kay, Academic Press, U.S. Pat. No. 5,625,048 to Tsien et al., issued Apr. 29, 1997; 5,777,079 to Tsien et al., issued Jul. 7, 1998; and U.S. Pat. No. 5,804,387 to Cormack et al., issued Sep. 8, 1998). In many cases these functional engineered fluorescent proteins have superior spectral properties to wild-type proteins and are preferred for use as reporter genes in the present invention. Preferred naturally fluorescent proteins include without limitation, EGFP, YFP, Renilla GFP and DS red.

As used herein, “the nuclear receptor super family” includes over 40 structurally and functionally similar proteins (aka, intracellular receptors, steroid receptors) as a large family. See, e.g., LeBlanc and Stunnenberg, 2 Genes & Development, 1811-1816 (1995).

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous. A structural gene (e.g., a HSV tk gene) which is operably linked to a polynucleotide sequence corresponding to a transcriptional regulatory sequence of an endogenous gene is generally expressed in substantially the same temporal and cell type-specific pattern as is the naturally-occurring gene.

As used herein, “orphan receptors” are members of the nuclear receptor superfamily for which no natural ligand (hormone) have yet been identified. Orphan receptors that may be substituted for preferred nuclear receptors in the methods of the present invention include, but are not limited to, various isoforms of HNF4 (Accession No. NM_{—}000457) (see, for example, Sladek et al., in Genes & Development 4:2353-2365 (1990)), the COUP family of receptors (e.g., COUP A (Accession No. NM_{—}005654) or COUP B (Accession No. NM_{—}021005); see, for example, Miyajima et al., in Nucleic Acids Research 16:11057-11074 (1988), Wang et al., in Nature 340:163-166 (1989), including the COUP-like receptors and COUP homologs, such as those described by Mlodzik et al., in Cell 60:211-224 (1990) and Ladias et al., in Science 251:561-565 (1991)), ultraspiracle (see, for example, Oro et al., in Nature 347:298-301 (1990) various isoforms of the orphan receptor NGFI-B, (Accession No. NM_{—}083884) see, for example, Milbrandt in Neuron 1:183-8 (1988) and Scearce et al., in J. Biol. Chem. 268:8855-8861 (1993)).

As applied to polypeptides, the term “substantial identity” means that two peptide sequences, when optimally aligned, such as by the programs GAP or BESTFIT using default gap weights, share at least 85 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “preferred nuclear receptors” refers to all mammalian species and splice isoforms of PPARα (SEQ. ID. No. 1), PPARβ (SEQ. ID. No. 2), PPARγ (SEQ. ID. No. 3), LXRα (SEQ. ID. No. 6), LXRβ (SEQ. ID. No. 7), FXR (SEQ. ID. No. 4), PXR (SEQ. ID. No. 5) and CAR (SEQ. ID. No. 8).

The term “parallel analysis” means that a test compound is analyzed and ranked or selected based on at least two activities in parallel. Parallel analysis can be accomplished using a single assay reaction that produces at least two discernable readouts, or via the use of two distinct (separate) assays that are run in parallel each of which produces a single readout. Parallel analysis requires that the assays are run at same or similar times (i.e. the same day) and under the same, or similar conditions, does not require that the assays or readouts be measured simultaneously.

A “reporter gene” includes any gene that directly or indirectly produces a specific reporter gene product, detectable label, enzymatic moiety, or cellular phenotype, such as drug resistance that can be used to monitor transcription of that gene. Preferred reporter genes include proteins with an enzymatic activity that provides enzymatic amplification of gene expression such as β-lactamase, luciferase, β-galactosidase, catalytic antibodies and alkaline phosphatase. Other reporter genes include proteins such as naturally fluorescent proteins or homologs thereof, cell surface proteins or the native or modified forms of an endogenous gene to which a specific assay exists or can be developed in the future. Preferred reporter genes for use in the present invention provide for multiplexed analysis.

As used herein, the term “modified host cell” refers to a eukaryotic cell, preferably a mammalian cell, which harbors a reporter polynucleotide, a nuclear receptor expression cassette, and a co-activator expression cassette and/or a co-repressor expression cassette. The reporter polynucleotide sequence may reside, in polynucleotide linkage, on the same polynucleotide as one or more of the expression cassette sequences, or may reside on a separate polynucleotide. The expression cassettes and/or the reporter polynucleotides may be present as an extrachromosomal element (e.g., replicon), may be integrated into a host cell chromosome, or may be transiently transfected in non-replicable, non-integrated form. Preferably, the expression cassettes and reporter polynucleotide are both stably integrated into a host cell chromosomal location, either by non-homologous integration or by homologous sequence targeting. Many cells can be used with the invention, including both primary and cultured cell lines derived from eukaryotic or prokaryotic cells. Such cells include, but are not limited to mammalian adult, fetal, or embryonic cells. These cells can be derived from the mesoderm, ectoderm, or endoderm and can be stem cells, such as embryonic or adult stem cells, or adult precursor cells. The cells can be of any lineage, such as vascular, neural, cardiac, fibroblasts, lymphocytes, hepatocytes, cardiac, hematopoeitic, pancreatic, epidermal, myoblasts, or myocytes. Other cells include baby hamster kidney (BHK) cells (ATCC No. CCL10), mouse L cells (ATCC No. CCLI.3), Jurkats (ATCC No. TIB 152) and 153 DG44 cells (see, Chasin (1986) Cell. Molec. Genet. 12: 555) human embryonic kidney (HEK) cells (ATCC No. CRL1573), Chinese hamster ovary (CHO) cells (ATCC Nos. CRL9618, CCL61, CRL9096), PC12 cells (ATCC No. CRL17.21) and COS-7 cells (ATCC No. CRL1651). Preferred established culture cell lines include Jurkat cells, CHO cells, neuroblastoma cells, P19 cells, F11 cells, NT-2 cells, and HEK 293 cells, such as those described in U.S. Pat. No. 5,024,939 and by Stillman et al. Mol. Cell. Biol. 5: 2051-2060 (1985).

“Treating” or “treatment” as used herein covers the treatment of a disease-state associated the nuclear receptor activity as disclosed herein, in a mammal, preferably a human, and includes:

(i) preventing a disease-state associated the nuclear receptor activity from occurring in a mammal, in particular, when such mammal is predisposed to the condition but has not yet been diagnosed as having it;

(ii) inhibiting a disease-state associated the nuclear receptor activity, i.e., arresting its development; or

(iii) relieving a disease-state associated the nuclear receptor activity, i.e., causing regression of the condition.

The term “transcription activation domain” is used herein refers to a protein, or protein domain with the capacity to enhance transcription of a structural sequence in trans; such enhancement may be contingent on the occurrence of a specific event, such as stimulation with an inducer and/or protein-protein interaction and may only be manifest in certain cell types. The ability to enhance transcription may affect the inducible transcription of a gene, or may effect the basal level transcription of a gene, or both. For example, a reporter polynucleotide may comprise a minimal-promoter driving transcription of a sequence encoding a reporter gene. Such a reporter polypeptide may be transferred to a nuclear receptor-responsive cell line for use in the creation of a modified host cell. Cloned sequences that silence expression of the reporter gene in cells cultured in the presence of a nuclear receptor agonist also may be included (e.g., to reduce basal transcription and ensure detectable inducibility). Numerous other specific examples of transcription regulatory elements, such as specific minimal promoters and response elements, are known to those of skill in the art and may be selected for use in the methods and polynucleotide constructs of the invention on the basis of the practitioner's desired application. Literature sources and published patent documents, as well as GenBank and other sequence information data sources can be consulted by those of skill in the art in selecting suitable transcription regulatory elements and other structural and functional sequences for use in the invention. Where necessary, a transcription regulatory element may be constructed by synthesis (and ligation, if necessary) of oligonucleotides made on the basis of available sequence information (e.g., GenBank sequences for a UAS, response element, minimal promoter etc).

Unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As used herein, the term “transcriptional regulatory sequence” refers to a polynucleotide sequence or a polynucleotide segment which, when placed in operable linkage to a transcribable polynucleotide sequence, can produce transcriptional modulation of the operably linked transcribable polynucleotide sequence. A positive transcriptional regulatory element is a DNA sequence which activates transcription alone or in combination with one or more other DNA sequences. Typically, transcriptional regulatory sequences comprise a promoter, or minimal promoter and frequently a hormone response element, and may include other positive and/or negative response elements as are known in the art or as can be readily identified by conventional transcription activity analysis (e.g., with “promoter trap” vectors, transcription rate assays, and the like). Often, transcriptional regulatory sequences include a promoter and a transcription factor recognition site and/or hormone response elements. The term often refers to a DNA sequence comprising a functional promoter and any associated transcription elements (e.g., enhancer, CCAAT box, TATA box, SP1 site, etc.) that are essential for transcription of a polynucleotide sequence that is operably linked to the transcription regulatory region. Enhancers and promoters include, but are not limited to, herpes simplex thymidine kinase promoter, cytomegalovirus (CMV) promoter/enhancer, SV40 promoters, pga promoter, regulatable promoters and systems (e.g., metallothionein promoter, the ecdysone promoter, the Tet on, Tet-off system, the PiP on/PIP off system, etc) adenovirus late promoter, vacinia virus 7.5 K promoter, and the like, as well as any permutations and variations thereof. Suitable hormone response elements include direct repeats (i.e., DRs; see Umesono et al., in Cell 65: 1255-1266 (1991), inverted repeats (i.e., IRs; see Umesono et al., in Nature 336: 262-265 (1988), and/or evereted repeats (ERs; see Baniahmad et al., in Cell 61: 505-514 of a degenerate Xn-AGGTCA (SEQ. ID. No. 23) core site. In direct repeats (DR, head to tail arrangement), the Xn sequence also serves as a gap which separates the two core binding sites. Thus, for example, spacers of 1, 3, 4 and 5 nucleaotides serve as preferred response elements for heterodimers of RXR with PPAR, VDR, T₃R and RAR respectively. The optimal gap length for each heterodimer is determined by protein-protein contacts which appropriately position the DNA binding domains of RXR and its partner.

As used herein, the phrase “system” refers to an intact organism or a cell-based system containing the various components required for analyzing co-factor interactions in response to the test compounds described herein, e.g., a preferred nuclear receptor, RXR as the heterotrimeric partner, co-factors, (co-activators, co-repressors, or fragments thereof) fused to a transcriptional modulators, co-factor-binding responsive reporter genes (which typically comprises a transcriptional regulatory sequence linked with a reporter gene, e.g., luciferase, chloramphenicol transferase, β-galactosidase, and the like). A “multiplexed system” refers to a single cell based system comprising at least two distinct reporter genes each of which is operatively linked to a distinct transcriptional regulatory sequence, which in turn are functionally coupled to either co-activator or co-repressor binding. The term also applies to a mixture of at least two distinct cell lines each of which comprises a single reporter gene operably linked to a transcriptional regulatory sequence and which is functionally coupled to a distinct effect of compound action, for example the binding of the co-activator to the nuclear receptor of interest in one cell type and the binding of the co-repressor to the nuclear receptor of interest in a second cell type.

The term “serial analysis” means that a test compound is analyzed and ranked based on a single activity. For example, compounds selected based solely on binding affinity, efficacy, ability to promote co-activator recruitment, ability to cause co-repressor dissociation or any other single factor, without reference to any other assay result or characteristic, are considered for the purposes here to be subject to “serial analysis”. A compound may be subject to multiple rounds of serial analysis, each round being based on data created from a single activity. For purposes here this analysis strategy is not considered to be equivalent to parallel analysis so long as each analysis or ranking step is completed independently of each other.

The following terms are used to describe the sequence relationships between two or more polynucleotides: “reference sequence”, “comparison window”, “sequence identity”, “percentage identical to a sequence”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods selected.

The term “sequence identity” means that two polynucleotide sequences are identical (i.e., on a nucleotide-by-nucleotide basis) over the window of comparison. The term “percentage identical to a sequence” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 90 percent sequence identity, preferably at least 95 percent sequence identity, as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least 90 percent sequence identity, preferably at least 95 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

Since the list of technical and scientific terms cannot be all encompassing, any undefined terms shall be construed to have the same meaning as is commonly understood by one of skill in the art to which this invention belongs. Reference to a “restriction enzyme” or a “high fidelity enzyme” may include mixtures of such enzymes and any other enzymes fitting the stated criteria, or reference to the method includes reference to one or more methods for obtaining cDNA sequences which will be known to those skilled in the art or will become known to them upon reading this specification.

Utility of the Compounds of the Invention

The compounds discovered using the methods of the present invention, have valuable pharmacological properties in mammals, and are particularly useful as cell type selective agonists, and partial agonists, for use in the treatment of diseases associated with defects in triglyceride transport, cholesterol transport, fatty acid and triglyceride metabolism, and cholesterol metabolism.

These diseases include, for example, hyperlipidemia, obesity, hyperglycemia, insulin resistance, diabetes, atherosclerosis (see, e.g., Patent Application Publication Nos. WO 00/57915 and WO 00/37077), excess lipid deposition in peripheral tissues such as skin (xanthomas) (see, e.g., U.S. Pat. Nos. 6,184,215 and 6,187,814), stroke, memory loss (Brain Research (1997), Vol. 752, pp. 189-196), optic nerve and retinal pathologies (i.e., macular degeneration, retintis pigmentosa), repair of traumatic damage to the central or peripheral nervous system (Trends in Neurosciences (1994), Vol. 17, pp. 525-530), prevention of the degenerative process due to aging (American Journal of Pathology (1997), Vol. 151, pp. 1371-1377), Parkinson's disease or Alzheimer's disease (see, e.g., International Patent Application Publication No. WO 00/17334; Trends in Neurosciences (1994), Vol. 17, pp. 525-530), prevention of degenerative neuropathics occurring in diseases such as diabetic neuropathies (see, e.g., International Patent Application Publication No. WO 01/82917), multiple sclerosis (Annals of Clinical Biochem. (1996), Vol. 33, No. 2, pp. 148-150), and autoimmune diseases (J. Lipid Res. (1998), Vol. 39, pp. 1740-1743).

Methods of using the compounds and pharmaceutical compositions of the invention are also provided herein. The methods involve both in vitro and in vivo uses of the compounds and pharmaceutical compositions for altering preferred nuclear receptor activity, in a cell type specific fashion.

In certain embodiments, the claimed methods involve the discovery and use of cell type specific compounds. In this context, a cell type specific compound typically exhibits at least a 10-fold difference in EC₅₀ or IC₅₀ for inducing gene expression in a preferred cell type compared to a full agonist, measured by at least one in vitro or in vivo assay described herein. Preferred cell types include any tissue or cell type for which a cell type specific effect is desired, examples include without limitation, macrophages, ileocytes, adipocytes, preadipocytes, myocytes, neuronal cells etc.

Methods for the treatment, prevention, or amelioration of one or more symptoms of diseases or disorder that are modulated by preferred nuclear receptor activity, including LXR α (SEQ. ID. No. 6) and/or LXR β (SEQ. ID. No. 7) as well as other orphan nuclear receptor activity, or in which preferred nuclear receptor activity, including LXR α (SEQ. ID. No. 6) and/or LXR β (SEQ. ID. No. 7) and/or orphan nuclear receptor activity, is implicated.

Also contemplated herein is the use of a compound of the invention, or a pharmaceutically acceptable derivative thereof, in combination with one or more of the following therapeutic agents in treating atherosclerosis: antihyperlipidemic agents, plasma HDL-raising agents, antihypercholesterolemic agents, cholesterol biosynthesis inhibitors (such as HMG CoA reductase inhibitors, such as lovastatin, simvastatin, pravastatin, fluvastatin, atorvastatin and rivastatin), acyl-coenzyme A:cholesterol acytransferase (ACAT) inhibitors, probucol, raloxifene, nicotinic acid, niacinamide, cholesterol absorption inhibitors, bile acid sequestrants (such as anion exchange resins, or quaternary amines (e.g., cholestyramine or colestipol)), low density lipoprotein receptor inducers, clofibrate, fenofibrate, benzofibrate, cipofibrate, gemfibrizol, vitamin B₆, vitamin B₁₂, anti-oxidant vitamins, β-blockers, FXR agonist, antagonist or partial agonist, anti-diabetes agents, angiotensin II antagonists, angiotensin converting enzyme inhibitors, platelet aggregation inhibitors, fibrinogen receptor antagonists, aspirin or fibric acid derivatives.

Compounds of the invention are preferably used in combination with a cholesterol biosynthesis inhibitor, particularly an HMG-CoA reductase inhibitor. The term HMG-CoA reductase inhibitor is intended to include all pharmaceutically acceptable salts, esters, free acid and lactone forms of compounds which have HMG-CoA reductase inhibitory activity and, therefore, the use of such salts, esters, free acids and lactone forms is included within the scope of this invention. Compounds which have inhibitory activity for HMG-CoA reductase can be readily identified using assays well-known in the art. For instance, suitable assays are described or disclosed in U.S. Pat. No. 4,231,938 and WO 84/02131. Examples of suitable HMG-CoA reductase inhibitors include, but are not limited to, lovastatin (MEVACOR®; see, U.S. Pat. No. 4,231,938); simvastatin (ZOCOR®; see, U.S. Pat. No. 4,444,784); pravastatin sodium (PRAVACHOL®; see, U.S. Pat. No. 4,346,227); fluvastatin sodium (LESCOL®; see, U.S. Pat. No. 5,354,772); atorvastatin calcium (LIPITOR®; see, U.S. Pat. No. 5,273,995) and rivastatin (also known as cerivastatin; see, U.S. Pat. No. 5,177,080). The structural formulas of these and additional HMG-CoA reductase inhibitors that can be used in combination with the compounds of the invention are described at page 87 of M. Yalpani, “Cholesterol Lowering Drugs,” Chemistry & Industry, pp. 85-89 (Feb. 5, 1996). In presently preferred embodiments, the HMG-CoA reductase inhibitor is selected from lovastatin and simvastatin.

The compounds of the present invention can also be used in methods for decreasing hyperglycemia and insulin resistance, i.e., in methods for treating diabetes. Diabetes mellitus, commonly called diabetes, refers to a disease process derived from multiple causative factors and characterized by elevated levels of plasma glucose, referred to as hyperglycemia. See, e.g., LeRoith, D. et al., (eds.), DIABETES MELLITUS (Lippincott-Raven Publishers, Philadelphia, Pa. U.S.A. 1996). According to the American Diabetes Association, diabetes mellitus is estimated to affect approximately 6% of the world population. Uncontrolled hyperglycemia is associated with increased and premature mortality due to an increased risk for macrovascular and macrovascular diseases, including nephropathy, neuropathy, retinopathy, hypertension, cerebrovascular disease and coronary heart disease. Therefore, control of glucose homeostasis is a critically important approach for the treatment of diabetes.

There are two major forms of diabetes: type 1 diabetes (formerly referred to as insulin-dependent diabetes or IDEM); and type 2 diabetes (formerly referred to as noninsulin dependent diabetes or NIDDM).

Type 2 diabetes is a disease characterized by insulin resistance accompanied by relative, rather than absolute, insulin deficiency. Type 2 diabetes can range from predominant insulin resistance with relative insulin deficiency to predominant insulin deficiency with some insulin resistance. Insulin resistance is the diminished ability of insulin to exert its biological action across a broad range of concentrations. In insulin resistant individuals, the body secretes abnormally high amounts of insulin to compensate for this defect. When inadequate amounts of insulin are present to compensate for insulin resistance and adequate control of glucose, a state of impaired glucose tolerance develops. In a significant number of individuals, insulin secretion declines further and the plasma glucose level rises, resulting in the clinical state of diabetes. Type 2 diabetes can be due to a profound resistance to insulin stimulating regulatory effects on glucose and lipid metabolism in the main insulin-sensitive tissues: muscle, liver and adipose tissue. This resistance to insulin responsiveness results in insufficient insulin activation of glucose uptake, oxidation and storage in muscle and inadequate insulin repression of lipolysis in adipose tissue and of glucose production and secretion in liver. In Type 2 diabetes, free fatty acid levels are often elevated in obese and some non-obese patients and lipid oxidation is increased.

Premature development of atherosclerosis and increased rate of cardiovascular and peripheral vascular diseases are characteristic features of patients with diabetes. Hyperlipidemia is an important precipitating factor for these diseases. Hyperlipidemia is a condition generally characterized by an abnormal increase in serum lipids, e.g., cholesterol and triglyceride, in the bloodstream and is an important risk factor in developing atherosclerosis and heart disease. For a review of disorders of lipid metabolism, see, e.g., Wilson, J. et al., (ed.), Disorders of Lipid Metabolism, Chapter 23, Textbook of Endocrinology, 9th Edition, (W. B. Sanders Company, Philadelphia, Pa. U.S.A. 1998). Hyperlipidemia is usually classified as primary or secondary hyperlipidemia. Primary hyperlipidemia is generally caused by genetic defects, while secondary hyperlipidemia is generally caused by other factors, such as various disease states, drugs, and dietary factors. Alternatively, hyperlipidemia can result from both a combination of primary and secondary causes of hyperlipidemia. Elevated cholesterol levels are associated with a number of disease states, including coronary artery disease, angina pectoris, carotid artery disease, strokes, cerebral arteriosclerosis, and xanthoma.

Dyslipidemia, or abnormal levels of lipoproteins in blood plasma, is a frequent occurrence among diabetics, and has been shown to be one of the main contributors to the increased incidence of coronary events and deaths among diabetic subjects (see, e.g., Joslin, E. Ann. Chim. Med. (1927), Vol. 5, pp. 1061-1079). Epidemiological studies since then have confirmed the association and have shown a several-fold increase in coronary deaths among diabetic subjects when compared with non-diabetic subjects (see, e.g., Garcia, M. J. et al., Diabetes (1974), Vol. 23, pp. 105-11 (1974); and Laakso, M. and Lehto, S., Diabetes Reviews (1997), Vol. 5, No. 4, pp. 294-315). Several lipoprotein abnormalities have been described among diabetic subjects (Howard B., et al., Arteriosclerosis (1978), Vol. 30, pp. 153-162).

The compounds of the invention can also be used effectively in combination with one or more additional active diabetes agents depending on the desired target therapy (see, e.g., Turner, N. et al., Prog. Drug Res. (1998), Vol. 51, pp.33-94; Haffner, S., Diabetes Care (1998), Vol. 21, pp. 160-178; and DeFronzo, R. et al. (eds.), Diabetes Reviews (1997), Vol. 5, No. 4). A number of studies have investigated the benefits of combination therapies with oral agents (see, e.g., Mahler, R., J. Clin. Endocrinol. Metab. (1999), Vol. 84, pp. 1165-71; United Kingdom Prospective Diabetes Study Group: UKPDS 28, Diabetes Care (1998), Vol. 21, pp. 87-92; Bardin, C. W.(ed.), CURRENT THERAPY IN ENDOCRINOLOGY AND METABOLISM, 6th Edition (Mosby—Year Book, Inc., St. Louis, Mo. 1997); Chiasson, J. et al., Ann. Intern. Med. (1994), Vol. 121, pp. 928-935; Coniff, R. et al., Clin. Ther. (1997), Vol.19, pp. 16-26; Coniff, R. et al., Am. J. Med. (1995), Vol. 98, pp. 443-451; Iwamoto, Y. et al., Diabet. Med. (1996), Vol. 13, pp. 365-370; Kwiterovich, P., Am. J. Cardiol (1998), Vol. 82 (12A), pp. 3U-17U). These studies indicate that diabetes and hyperlipidemia modulation can be further improved by the addition of a second agent to the therapeutic regimen.

Accordingly, the compounds of the invention may be used in combination with one or more of the following therapeutic agents in treating diabetes: sulfonylureas (such as chlorpropamide, tolbutamide, acetohexamide, tolazamide, glyburide, gliclazide, glynase, glimepiride, and glipizide), biguanides (such as metformin), thiazolidinediones (such as ciglitazone, pioglitazone, troglitazone, and rosiglitazone), and related insulin sensitizers, such as selective and non-selective activators of PPARα, PPARβ and PPARγ; dehydroepiandrosterone (also referred to as DHEA or its conjugated sulphate ester, DHEA-SO₄); FXR agonists, antagonists or partial agonists, antiglucocorticoids; TNF α inhibitors; α-glucosidase inhibitors (such as acarbose, miglitol, and voglibose), pramlintide (a synthetic analog of the human hormone amylin), other insulin secretogogues (such as repaglinide, gliquidone, and nateglinide), insulin, as well as the therapeutic agents discussed above for treating atherosclerosis.

Further provided by this invention are methods of using the compounds of the invention to treat obesity, as well as the complications of obesity. Obesity is linked to a variety of medical conditions including diabetes and hyperlipidemia. Obesity is also a known risk factor for the development of type 2 diabetes (See, e.g., Barrett-Conner, E., Epidemol. Rev. (1989), Vol. 11, pp. 172-181; and Knowfer, et al., Am. J Clin. Nutr. (1991), Vol. 53, pp. 1543-1551).

In addition, the compounds of the invention can be used in combination with agents used in treated obesity or obesity-related disorders. Such agents, include, but are not limited to, phenylpropanolamine, phentermine, diethylpropion, mazindol, fenfluramine, dexfenfluramine, phentiramine, β₃ adrenoceptor agonist agents; sibutramine, gastrointestinal lipase inhibitors (such as orlistat), and leptins. Other agents used in treating obesity or obesity-related disorders include neuropeptide Y, enterostatin, cholecytokinin, bombesin, amylin, histamine H₃ receptors, dopamine D₂ receptors, melanocyte stimulating hormone, corticotrophin releasing factor, galanin and gamma amino butyric acid (GABA).

### Screening Methods

Those of skill in the art recognize that methods to determine the formation or disruption of nuclear receptor complexes contemplated by the invention can be carried out in a wide variety of ways. In one embodiment, compounds of the present invention will be identified by A) pre-screening a library of compounds to identify those that exhibit high affinity binding to the receptor; B) Taking those compounds that exhibit high affinity binding and determining the relative degree of co-activator and co-repressor recruitment, and C) selecting those compounds that exhibit substantial co-repressor dissociation without substantially increased co-activator recruitment.

Preferably in this case the test compounds can be pre-selected from a library to exhibit an EC₅₀ or IC₅₀ of 100 nM or less for the nuclear receptor of interest in one of the in vivo or in vitro assays described herein.

Alternatively in another embodiment the compounds of the present invention will be identified by screening the test compounds using one of the multiplexed methods described herein without prescreening the test compounds for affinity.

As readily recognized by those of skill in the art, a wide variety of test compounds and known scaffolds can be employed in the invention assays. Examples of the classes of compounds contemplated for use in the practice of the present invention include, but are not limited to, steroids, sterols, retinoids, prostaglandins, leukotrienes, thiazolidinediones, farnesoids, aminobenzoates, hydroxybenzoates, eicosanoids, cholesterol metabolites, fibrates, amino acids, sugars, nucleotides, fatty acids, lipids, serotonin, dopamine, catecholamines, acid azoles, and the like.

In a particular aspect, the plurality of test compounds employed in the invention assays can comprise a combinatorial library of peptide or small molecule compounds, wherein each individual test compound is one of an array of structurally related compounds. See, eg., Bunin, B. A. N. Ellman, J. A., J. Am. Chem. Soc. 114:10997-10998 (1992) and references contained therein. Preferably, test compounds identified as modulators will be of low molecular weight (less than 10,000 Daltons, preferably less than 5,000, and most preferably less than 1,000) which can be readily formulated as useful therapeutic agents. Preferably such a library will be based upon scaffolds of known nuclear receptor activity or affinity, for example those described in U.S. Pat. No. 6,316,503, U.S. Pat. No. 6,452,032, PCT publications WO 01/60818, WO 02/72598, WO 00/37077; and U.S. applications US2002/72073, US2002/132223 and US2002/120137.

Suitable cell based assays for prescreening test compounds include, but are not limited to, the co-transfection assay, the use of LBD-Gal4 chimeras and protein-protein interaction assays (see, for example, Lehmann. et al., J. Biol Chem. (1997), Vol. 272, No. 6, pp. 3137-3140).

In addition many biochemical screening formats exist for prescreening compound libraries to identify high affinity ligands which include, but are not limited to, direct binding assays, ELISAs, fluorescence polarization assays, FRET and Time resolved FRET based coactivator recruitment assays (see, generally, Glickman et al., J. Biomolecular Screening (2002), Vol. 7, No. 1, pp. 3-10).

High throughput screening systems are commercially available (see, e.g., Zymark Corp., Hopkinton, Mass.; Air Technical Industries, Mentor, Ohio; Beckman Instruments Inc., Fullerton, Calif.; Precision Systems, Inc., Natick, Mass.) that enable these assays to be run in a high throughput mode. These systems typically automate entire procedures, including all sample and reagent pipetting, liquid dispensing timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Assays that do not require washing or liquid separation steps are preferred for such high throughput screening systems and include biochemical assays such as fluorescence polarization assays (see, for example, Owicki, J., Biomol. Screen (October 2000), Vol. 5, No. 5, pp. 297), scintillation proximity assays (SPA) (see, for example, Carpenter et al., Methods Mol. Biol. (2002), Vol 190, pp. 31-49) and fluorescence resonance energy transfer energy transfer (FRET) or time resolved FRET based coactivator recruitment assays (Mukherjee et al., J. Steroid Biochem. Mol. Biol. (July 2002); Vol. 81, No. 3, pp. 217-25; (Zhou et al., Mol. Endocrinol. (October 1998), Vol. 12, No. 10, pp. 1594-604). Preferred methods for use in the present invention utilize multiplexed systems that enable at least two parameters to be simultaneously measured.

Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y. L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter, a fluorescent microscope or a fluorescent plate reader. In general, all of these systems have an excitation light source which can be manipulated to create a light source with a defined wavelength maxima and band width which passes through excitation optics to excite the sample.

Typically the excitation wavelength is designed to selectively excite the fluorescent sample within its excitation or absorption spectrum. For most FRET based assays the excitation wavelength is usually selected to enable efficient excitation of the donor while minimizing direct excitation of the acceptor. In response the sample (if fluorescent) emits radiation that has a wavelength that is different from the excitation wavelength. Collection optics then collect the emission from the sample, and direct it to one or more detectors, such as photomultiplier tubes or CCD cameras. Preferably the detector will include a filter to select specific wavelengths of light to monitor. For time resolved applications, for example time resolved FRET, the excitation and or emission optical paths include control mechanisms to precisely terminate illumination and then to wait for a precise period of time before collecting emitted light. By using compounds such as lanthanides that exhibit relatively long-lived light emission it is possible to gain significant enhancements in detection sensitivity and accuracy.

The detection devices can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation.

Suitable instrumentation for fluorescence microplate readers include without limitation the CytoFluor™ 4000 available from PerSeptive Biosystems. For 96-well based assays black walled plates with clear bottoms, such as those manufactured by Costar are preferred.

Suitable instrumentation for luminescence measurements include standard liquid scintillation plate readers, including without limitation the Wallac Microbeta or equivalents commercially available from Packard, Perkin Elmer and a number of other manufactures.

### Assay Methods

If a fluorescently labeled ligand is available, fluorescence polarization assays provide a way of detecting binding of compounds to the nuclear receptor of interest by measuring changes in fluorescence polarization that occur as a result of the displacement of a trace amount of the label ligand by the compound. Additionally this approach can also be used to monitor the ligand dependent association of a fluorescently labeled coactivator peptide to the nuclear receptor of interest to detect ligand binding to the nuclear receptor of interest.

Many suitable fluorescent labeling reagents are commercially available from Molecular Probes. See Haughland (2002) Handbook of Fluorescent Probes and Research Products, 9th edition, published by Molecular Probes, Inc. Suitable fluorescent labels for use as detectable labels herein include without limitation, fluorescein and its derivatives such as fluoresceinamine, carboxyfluorescein, iodoacetamidofluorescein, aminomethylfluorescein, alkylaminomethylfluorescein, fluorescein isothiocyanate (FITC), dichlorotriazinyl aminofluorescein (DTAF), 4-chloro-6-methoxy-1,3,5-triazin-2-yl-aminofluorescein, and fluorinated fluorescein such as Oregon Green®. Other suitable labels include rhodamine and their derivatives or analogs such as tetramethyl rhodamine, carboxytetramethylrhodamine, Lissamine™ Rhodamine B, Texas Red®, carboxy-X-rhodamine and Rhodamine Red™-X. Other suitable labels include cyanine dyes such as Cy3™ and Cy5™ and the Alexa Fluor® dyes 488, 532, 546, 555, 568, 594, 633, 660 and 680. Other useful fluorescent labeling groups include coumarin and its derivatives, such as the Alexa Fluor® dyes 350 and 430. Other useful fluorescent labeling groups are indacenes and indacene derivatives such as the BODIPY® series of dyes and rosamine or rosamine derivatives such as tetramethylrosamine and chloromethyl-X-rosamine. Suitable fluorescent labels for use in multiplexed analysis include without limitation fluorescein and rhodamine, fluorescein and coumarin, and rhodamine and coumarin etc.

The ability of a compound to bind to a receptor, or heterodimer complex with RXR, can also be measured in a homogeneous assay format by assessing the degree to which the compound can compete off a radiolabelled ligand with known affinity for the receptor using a scintillation proximity assay (SPA). In this approach, the radioactivity emitted by a radiolabelled compound (for example, a radiolabelled ligand) generates an optical signal when it is brought into close proximity to a scintillant such as a Ysi-copper containing bead, to which the nuclear receptor is bound. If the radiolabelled compound is displaced from the nuclear receptor the amount of light emitted from the nuclear receptor bound scintillant decreases, and this can be readily detected using standard microplate liquid scintillation plate readers such as, for example, a Wallac MicroBeta reader.

The heterodimerization of a nuclear receptor can also be measured by fluorescence resonance energy transfer (FRET), or time resolved FRET, to monitor the ability of the compounds provided herein to bind to the nuclear receptor. Both approaches rely upon the fact that energy transfer from a donor molecule to an acceptor molecule only occurs when donor and acceptor are in close proximity. Typically the purified LBD of the nuclear receptor of interest is labeled with biotin then mixed with stoichiometric amounts of lanthanide labeled streptavidin (Wallac Inc.), and the purified LBD of RXR, or alternate heterodimer, is labeled with a suitable fluorophore such as CY5™. Equimolar amounts of each modified LBD are mixed together and allowed to equilibrate for at least 1 hour prior to addition to either variable or constant concentrations of the test compound for which the activity is to be determined. After equilibration, the time-resolved fluorescent signal is quantitated using a fluorescent plate reader. The activity of the test compound can then be estimated from a plot of fluorescence versus concentration of test compound added.

This approach can also be exploited to measure the ligand dependent interaction of a co-activator peptide with a nuclear receptor in order to characterize the agonist or antagonist activity of the compounds disclosed herein. Typically the assay in this case involves the use a recombinant epitope, or affinity tagged nuclear receptor ligand binding domain (LBD) fusion protein and a synthetic biotinylated peptide derived from the receptor interacting domain of a co-activator peptide such as the steroid receptor coactivator 1 (SRC-1) (SEQ. ID. No. 11). Typically the tagged-LBD is labeled with a lanthanide chelate such as europium (Eu), via the use of antibody specific for the tag, and the co-activator peptide is labeled with allophycocyanin via a streptavidin-biotin linkage.

In the presence of an agonist for the nuclear receptor, the peptide is recruited to the tagged-LBD bringing europium and allophycocyanin into close proximity to enable energy transfer from the europium chelate to the allophycocyanin. Upon excitation of the complex with light at 340 nm excitation energy absorbed by the europium chelate is transmitted to the allophycocyanin moiety resulting in emission at 665 nm. If the europium chelate is not brought in to close proximity to the allophycocyanin moiety there is little or no energy transfer and excitation of the europium chelate results in emission at 615 nm. Thus the intensity of light emitted at 665 nm gives an indication of the strength of the protein-protein interaction. The activity of a nuclear receptor antagonist can be measured by determining the ability of a compound to competitively inhibit (i.e., IC₅₀) the activity of an agonist for the nuclear receptor.

In addition, a variety of cell based assay methodologies may be successfully used in prescreening assays to identify and profile the affinity of compounds of the present invention. These approaches include the co-transfection assay, translocation assays, complementation assays and the use of gene activation technologies to over express endogenous nuclear receptors.

Three basic variants of the co-transfection assay strategy exist, co-transfection assays using full-length nuclear receptor, co transfection assays using chimeric nuclear receptors comprising the ligand binding domain of the nuclear receptor of interest fused to a heterologous DNA binding domain, and assays based around the use of the mammalian two hybrid assay system.

The basic co-transfection assay is based on the co-transfection into the cell of an expression plasmid to express the nuclear receptor of interest in the cell with a reporter plasmid comprising a reporter gene whose expression is under the control of DNA sequence that is capable of interacting with that nuclear receptor. (See for example U.S. Pat. Nos. 5,071,773; 5,298,429 and 6,416,957). Treatment of the transfected cells with an agonist for the nuclear receptor increases the transcriptional activity of that receptor which is reflected by an increase in expression of the reporter gene which may be measured by a variety of standard procedures.

In one embodiment of this method the host cell endogenously expresses the nuclear receptor heterodimer (typically with RXR) and appropriate co-factors. Typically such a situation may occur with a primary cell or cell lines derived directly from a primary cell type, such as, for example when a macrophage cell is used in the present invention. Accordingly creation of a multiplexed system requires the transfection into the cell of a suitable reporter gene(s) as are described herein. Alternatively the expression of endogenous gene can be used to monitor co-activator and co-repressor recruitment in response to the addition of a test compound.

In another aspect the host cell may lack sufficient endogenous expression of a suitable nuclear receptor, in which case one may be introduced by transfection of the cell line with an expression plasmid, as described below.

Typically, the expression plasmid comprises: (1) a promoter, such as an SV40 early region promoter, HSV tk promoter or phosphoglycerate kinase (pgk) promoter, CMV promoter, Srα promoter or other suitable control elements known in the art, (2) a cloned polynucleotide sequence, such as a cDNA encoding a receptor, co-factor, or fragment thereof, ligated to the promoter in sense orientation so that transcription from the promoter will produce a RNA that encodes a functional protein, and (3) a polyadenylation sequence. For example and not limitation, an expression cassette of the invention may comprise the cDNA expression cloning vectors, or other preferred expression vectors known and commercially available from vendors such as Invitrogen, Carlsbad, Calif., Stratagene, San Diego, Calif. or Clontech, Palo Alto, Calif. etc. The transcriptional regulatory sequences in an expression cassette are selected by the practitioner based on the intended application; depending upon the specific use, transcription regulation can employ inducible, repressible, constitutive, cell-type specific, developmental stage-specific, sex-specific, or other desired type of promoter or control sequence.

Alternatively, the expression plasmid may comprise an activation sequence to activate or increase the expression of an endogenous chromosomal sequence. Such activation sequences include for example, a synthetic zinc finger motif (for example see U.S. Pat. Nos. 6,534,261 and 6,503,7171) or a strong promoter or enhancer sequence together with a targeting sequence to enable homologous or non-homologous recombination of the activating sequence upstream of the gene of interest.

In one embodiment, full-length genes encoding the complete cDNA sequence of the nuclear receptor or co-factor are used herein.

In another embodiment of this method chimeras of these full-length genes are used in place of the full-length nuclear receptor. Such chimeras typically comprise the ligand binding domain (LBD) of the nuclear receptor of interest coupled to a heterologous DNA binding domain (DBD).

In the case of human LXR α (SEQ. ID. No. 6) the LBD comprises amino acids 188-447 for LXR β (SEQ. ID. No. 7) the LDB comprises amino acids 198-461, for FXR (SEQ. ID. No. 4), the LBD comprises amino acids 244 to 472 of the full-length sequence, for CAR (SEQ. ID. No. 8), the LBD comprises amino acids 229-414, and for type 1 PXR (SEQ. ID. No. 5), the LBD comprises amino acids 857-1039.

Typically for such chimeric constructs, heterologous DNA binding domains from distinct, well-defined nuclear receptors are used, for example including without limitation, the DBDs of the glucocorticoid receptor, GR (accession no. NM_{—}000176)(amino acids 421-486), mineralocorticoid receptor, MR (accession no. NM_{—}055775) (amino acids 603-668), androgen receptor, AR (accession no XM_{—}010429NM_{—}055775) (amino acids 929-1004), progesterone receptor, PR (amino acids 622-695), and estrogen receptor alpha, ERα (accession no. XM_{—}045967) (amino acids 185-250).

Alternatively DNA binding domains from yeast or bacterially derived transcriptional regulators such as members of the GAL 4 and Lex A/Umud super families may be used.

GAL4 (GenBank Accession Number P04386, SEQ. ID. No. 17) is a positive regulator for the expression of the galactose induced genes. The DNA binding domain of the yeast Gal4 protein comprises at least the first 74 amino acids of SEQ. ID. NO.17 (see for example, Keegan et al., Science 231: 699-704 (1986). Preferably for use in the present invention, the first 96 amino acids of the Gal4 protein (SEQ. ID. NO.17) are used, most preferably the first 147 amino acid residues of yeast Gal4 protein (SEQ. ID. NO.17) are used.

Full length LEXA (GenBank accession number ILEC, (SEQ. ID. NO.18)) is composed of a structurally distinct N-terminal DNA binding domain and a C-terminal catalytic domain separated by a short hydrophilic hinge region. The DNA binding domain residues (1 to 69) contains 3 alpha helices followed by 2 anti-parallel beta strands. Members of the LEXA family repress a number of genes involved in the response to DNA damage including the RecA and LexA proteins themselves. For use in the present invention, preferably the first 70 or more amino acids of the LexA protein (SEQ. ID. NO.18) are used. Most preferably the first 74 amino acid residues of LexA protein (SEQ. ID. NO.18) are used.

For those receptors that function as heterodimers with RXR, such as the LXRs, the method typically includes the use of expression plasmids for both the nuclear receptor of interest and RXR. Such sequences include, but are not limited to the following members of the RXR gene family, including RXRα, (SEQ. ID. No. 24) GenBank Accession No. NM_{—}002957, RXRβ (SEQ. ID. No. 25) GenBank Accession No. XM_{—}042579 and RXRγ (SEQ. ID. No. 26) GenBank Accession No. XM_{—}053680.

Reporter polynucleotides may be constructed using standard molecular biological techniques by placing cDNA encoding for the reporter gene downstream from a suitable minimal promoter. For example luciferase reporter plasmids may be constructed by placing cDNA encoding firefly luciferase immediately down stream from the herpes virus thymidine kinase promoter (located at nucleotides residues-105 to +51 of the thymidine kinase nucleotide sequence) which is linked in turn to the various response elements.

Response elements contemplated for use in the practice of the present invention are well known and have been thoroughly described in the art. Such response elements can include direct repeat structures or inverted repeat structures based on well defined hexad half sites, as described in greater detail below. Exemplary hormone response elements are composed of at least one direct repeat of two or more half sites, separated by a spacer having in the range of 0 up to 6 nucleotides. The spacer nucleotides can be randomly selected from any one of A, C, G or T. Each half site of response elements contemplated for use in the practice of the invention comprises the sequence: -RGBNNM- (SEQ. ID. No. 22), wherein R is selected from A or G; B is selected from G, C, or T; each N is independently selected from A, T, C, or G; and M is selected from A or C; is with the proviso that at least 4 nucleotides of said -RGBNNM- -(SEQ. ID. No. 22) sequence are identical with the nucleotides at corresponding positions of the sequence -AGGTCA- -(SEQ. ID. No. 23). Response elements employed in the practice of the present invention can optionally be preceded by N, wherein x falls in the range of 0 up to 5. A preferred response element useful in the methods of the present invention is a direct repeat of the nucleotide sequence AGGTCA -(SEQ. ID. No. 23) separated by 4 nucleotides.

The choice of hormone response element is dependent upon the type of multiplexed system to be used. In the case of the use of the full length LXR the LXR RE would typically be used. In the case of a LXR-LBD-Gal4 fusion, a GAL4 UAS would be used and in the case of the LXR-LBD-LexA fusion a Lex A UAS would be used. These constructs are described in more detail in Table 2, below.

TABLE 2

| Reporter Gene | | |
|---|---|---|
| Construct | Response Element (RE) | Nuclear Receptor |
| LXRE 3 X RE | 5′GGTTTA-NNNN-AGTTCA-3′ | Full length LXR |
| | (SEQ. ID. No. 27) | |
| GAL4-UAS 4 X RE | 5′CGGRNNRCYNYNCNCCG-3′ | LBD-Gal4 chimeras |
| | (SEQ. ID. No. 28) | |
| | where Y = C or T, R = A or G, | |
| | and N = A, C, T or G | |
| LEX A-UAS x 4 | (1) 5′-CGAACNNNNGTTCG-3′ | LBD-Lex A chimeras |
| | (2) (SEQ. ID. No. 29). | |

Numerous reporter gene systems are known in the art and include, for example, alkaline phosphatase (see, Berger, J., et al., Gene (1988), Vol. 66, pp. 1-10; and Kain, S. R., Methods. Mol. Biol. (1997), Vol. 63, pp. 49-60), β-galactosidase (See, U.S. Pat. No. 5,070,012, issued Dec. 3, 1991 to Nolan et al., and Bronstein, I., et al., J. Chemilum. Biolum. (1989), Vol. 4, pp. 99-111), chloramphenicol acetyltransferase (See, Gorman et al., Mol. Cell Biol. (1982), Vol. 2, pp. 1044-51), β-glucuronidase, peroxidase, β-lactamase (U.S. Pat. Nos. 5,741,657 and 5,955,604), catalytic antibodies, luciferases (U.S. Pat. Nos. 5,221,623; 5,683,888; 5,674,713; 5,650,289; and 5,843,746) and naturally fluorescent proteins (Tsien, R. Y., Annu. Rev. Biochem. (1998), Vol. 67, pp. 509-44).

Virtually of the above reporter gene systems may be used for multiplexed analysis. Preferred systems for multiplexed analysis include, but are not limited to, luciferase and β-galactosidase, luciferase and β-lactamase and luciferase and alkaline phosphatase.

Numerous methods of co-transfecting the expression and reporter plasmids are known to those of skill in the art and may be used for the co-transfection assay to introduce the plasmids into a suitable cell type.

These pre-screening approaches enable the selection of test compounds that interaction with the nuclear receptor of interest with high affinity. Preferably such pre-selected compounds exhibit an affinity, as measured via any of the methods disclosed herein, of at least 500 nM, preferably at least 300 nM, more preferably at least 200 nM, and most preferably at least 100 nM.

### Co-Factor Interaction Assays

To identify compounds that act selectively on co-activator or co-repressor binding a mammalian two-hybrid assay can be used (see, for example, U.S. Pat. Nos. 5,667,973, 5,283,173 and 5,468,614). This approach identifies protein-protein interactions in vivo through reconstitution of a strong transcriptional activator upon the interaction of two proteins, a “bait” and “prey” (Fields S and Song O (1989) Nature 340: 245).

The method enables the interaction of the nuclear receptor with the co-activator and co-repressor to be coupled to distinct transcriptional readouts, enabling the selection of compounds that preferentially modify the interactions of the receptor with either a co-activator or (and) co-repressor compared to a full agonist. In one embodiment the method is set up so that expression of a first reporter gene is dependent on co-activator recruitment, while expression of a second reporter gene is dependent on co-repressor recruitment.

The method may be preformed within either a single modified host cell comprising two reporter genes, or two distinct modified host cells, each or which contains a single reporter gene, which can be mixed together to provide for a multiplexed readout.

Thus treatment of the modified host cells with a test compound that causes both increased co-activator recruitment and decreased co-repressor recruitment will cause an increase in expression of the first reporter gene, and a decrease in expression, or no change, in the expression of the second reporter gene. Conversely, treatment of the modified host cells with a test compound that causes increased co-repressor recruitment, and decreased co-activator recruitment will cause a decrease in constitutive or basal expression of the first reporter gene expression, and increase the expression of the second reporter gene. Thus compounds can be identified and selected that exhibit specific effects on co-activator and co-repressor recruitment.

Accordingly in one embodiment, the present invention includes a method to identify compounds that bind to a nuclear receptor and exhibit cell type specific actions, said method comprising:

a) contacting a modified host cell with a test compound, wherein said modified host cell comprises:

i) a first fusion, comprising the co-activator, fused to a first heterologous DNA binding domain,

ii) a second fusion protein comprising the co-repressor, fused to a second heterologous DNA binding domain,

iii) a third fusion protein comprising the ligand binding domain of the nuclear receptor of interest fused to a transcription activation domain,

iv) a first reporter gene operably linked to a first transcriptional regulatory sequence specific for said first heterologous DNA binding domain,

v) a second reporter gene operably linked to a second transcriptional regulatory sequence specific for said second heterologous DNA binding domain,

b) identifying those test compounds which cause altered expression of said first reporter gene product and similar, or altered expression of said second reporter gene product compared to a control modified host cell.

In one embodiment of this method, the method further comprises the step of adding the test compound in the presence of a known antagonist, and identifying those compounds which caused reduced expression of said second reporter gene product without increasing expression of said first reporter gene.

In one embodiment of this method, the method further comprises the step of adding the test compound in the presence of a known agonist, and identifying those compounds which caused reduced expression of said first reporter gene product without increasing expression of said second reporter gene.

In another embodiment of this method, the method further comprises the step of prescreening the test compounds to determine their affinity for the nuclear receptor of interest. In one aspect, the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

As used herein, the term “coactivator” or “co-activator” means any limiting protein or peptide factor that binds to the nuclear receptor via direct protein-protein contact(s), in an agonist-dependent manner. Coactivators typically contain at least one interaction domain motif typically conforming to the sequence -LXXLL- (SEQ. ID. No. 9). Coactivators typically bind to nuclear receptors as a result of conformational changes in the LBD that result in the exposure of a high affinity interaction domain for the co-activator.

Co-activators contemplated for use in the practice of the present invention include, but are not limited to, SRC-1 (aka NcoA-1) (SEQ. ID. No. 11) (See, e.g., Onate et al., in Science 270:1354-1357 (1995)), TIF2 (SEQ. ID. No. 13) (aka GRIP1, SRC-2)(See, e.g., LeDouarin et al., in EMBO Journal 14:2020-2033 (1995) and Baur et al., in EMBO Journal 15:110-124 (1996)), TRIP1 (SEQ. ID. No. 30) (See, e.g., Lee et al., in Nature 374:91-94 (1995)), RIP140 (SEQ. ID. No. 31) (See, e.g., Cavailles et al., in EMBO Journal 14:3741-3751 (1995)), ERAP (SEQ. ID. No. 32) (See, e.g., Halachmi et al., in Science 264:1455-1458 (1994)), CBP (SEQ. ID. No. 33), p300 (SEQ. ID. No. 34), p/CIP (SEQ. ID. No. 35) (aka AIB-1, ACTR, RAC, TRAM-1, SCR-3), SWI (SEQ. ID. No. 36) (aka SNF), GCN5 (SEQ. ID. No. 37), P/CAF (SEQ. ID. No. 38), PGC-1 (SEQ. ID. No. 39), PGC-2 (SEQ. ID. No. 40), ARA70 (SEQ. ID. No. 41), TRAP250 (SEQ. ID. No. 12) (aka DRIP, ARC), and analogs thereof See, generally, Rosenfeld and Glass, 276 J. Biol. Chem., 3686-3688 (2001) and Glass and Rosenfeld, 14 Genes & Development, 121-141 (2000).

Co-activators can include fragments of the above co-factors as well as the full-length protein. The selection of particular fragments is well known in the art. For example such fragments will typically comprise at least one interaction domain (LXXLL SEQ ID. No. 9) and about 5 to 20 amino acids, derived from a full length co-activator sequence, immediately N-terminal of the interaction domain, and about 5 to 10 amino acids, derived from the co-activator sequence, immediately C-terminal of the interaction domain.

Preferred co-activators useful in the methods of the present invention include without limitation peptides derived from SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39) and PGC-2 (SEQ. ID. No. 40).

Alternatively coactivators can include amino acid sequences which are not found in nature but which are identified by a peptide screening method as binding to a LBD of a nuclear receptor in an agonist-dependent manner. Typically such sequences will exhibit substantial identity to the corresponding sequences of known co-activators.

Preferred co-activators of this type include, without limitation, those peptide sequences listed below in Table 3.

TABLE 3

| | |
|---|---|
| Co-activator Peptide Sequence | Sequence ID listing |

| | |
|---|---|
| CPSSHSSLTERHKILHRLLQEGSPS | SEQ. ID. No. 46 |
| KYSQTSHKLVQLLTTTAEQQ | SEQ. ID. No. 47 |
| SLTARHKILHRLLQEGSPSD | SEQ. ID. No. 48 |
| KESKDHQLLRYLLDKDEKDL | SEQ. ID. No. 49 |
| HDSKGQTLLQLLTTKADQM | SEQ. ID. No. 50 |
| SLKEKHKILHRLLQDSSSPV | SEQ. ID. No. 51 |
| PKKKENALLRYLLDKDDTKD | SEQ. ID. No. 52 |
| LESKGHKKLLQLLTCSSDDR | SEQ. ID. No. 53 |
| LLQEKHRILHKLLQNGNSPA | SEQ. ID. No. 54 |
| KKKENNALLRYLLDRDDPSD | SEQ. ID. No. 55 |
| SKVSQNPILTSLLQITGNGG | SEQ. ID. No. 56 |
| GNTKNHPMLMNLLKDNPAQD | SEQ. ID. No. 57 |
| DAASKHKQLSELLRGGSGSS | SEQ. ID. No. 58 |
| DAASKHKQLLRYLLRGGSGSS | SEQ. ID. No. 59 |
| DAASKHKQLSELLDKDEKDL | SEQ. ID. No. 60 |
| DAASKHKLLRYLLDKDEKDL | SEQ. ID. No. 61 |
| KESKDHQLSELLDKDEKDL | SEQ. ID. No. 62 |
| KESKDHQLLRYLLRGGSGSS | SEQ. ID. No. 63 |
| KESKDHQLSELLRGGSGSS | SEQ. ID. No. 64 |
| KESKKHKQLRYLLRGGSGSS | SEQ. ID. No. 65 |
| DAASDHQLLRYLLRGGSGSS | SEQ. ID. No. 66 |
| KESKDHQLLRYLLDKGSGSS | SEQ. ID. No. 67 |
| KESKDHQLLRYLLRGDEKDL | SEQ. ID. No. 68 |
| KESKDHQLLRYLLRGGEKDL | SEQ. ID. No. 69 |
| KESKDHQLLRYLLRKDEKDL | SEQ. ID. No. 70 |
| DAASKHKLLRYLLRGGSGSS | SEQ. ID. No. 71 |
| KESKKHQLLRYLLRGGSGSS | SEQ. ID. No. 72 |
| KESKDHKLLRYLLRGGSGSS | SEQ. ID. No. 73 |
| KESKDHQQLRYLLRGGSGSS | SEQ. ID. No. 74 |
| KESKDHQLLSYLLRGGSGSS | SEQ. ID. No. 75 |
| KESKDHQLLRELLRGGSGSS | SEQ. ID. No. 76 |
| KESKDHQQLRYLLDKDEKDL | SEQ. ID. No. 77 |
| DAASKHKLLSELLRGGSGSS | SEQ. ID. No. 78 |
| DAASKHKLLRYLLDRGGSGSS | SEQ. ID. No. 79 |
| DAASKHKQLSELLDGGSGSS | SEQ. ID. No. 80 |
| KESKDHQLLRYLLRKDEKDL | SEQ. ID. No. 81 |
| GYVNADLNYLLGSASTF | SEQ. ID. No. 82 |
| GDDDNPLITLLTGAHSY | SEQ. ID. No. 83 |
| IANNALLYALLSDHGAH | SEQ. ID. No. 84 |
| IGCTSALSRLLINYGDL | SEQ. ID. No. 85 |

As used herein, the term “corepressor” means any limiting protein or peptide factor that binds to the unoccupied, or antagonist bound nuclear receptor via direct protein-protein contact(s),and in which dissociates from the nuclear receptor in an agonist dependent manner. Co-repressors typically comprise at least one interacting domain of general form LXXI/HIXXX(I/L)(SEQ. ID. No.10).

Co-repressors contemplated for use in the practice of the present invention include, but are not limited to, SMRT (SEQ. ID. No. 14) (aka TRAC2) and N-CoR (SEQ. ID. No. 15) (aka RIP13)(See, e.g., Kurokawa et al., in Nature 377:451-454 (1995); Chen and Evans, 377 Nature, 454-457 (1995); Chen et al., 93 PNAS, 7567-7571 (1996); Horlein et al., 377 Nature, 397-404 (1995); and Sande and Privalsky, 10 Mol. Endo., 813-825 (1996)), ALIEN (SEQ. ID. No. 37) (See, e.g., Dressel et al., Molecular and Cellular Biology, 3383-3394 (1999)); Hairless (SEQ. ID. No. 34) (See, e.g., Potter et al, 15 Genes and Development, 2687-2701 (2001)) SUN-CoR (SEQ. ID. No. 42) (See Zamir et al., Proc. Natl. Acad. Sci. 94 and analogs thereof. See, generally, Rosenfeld and Glass, 276 J. Biol. Chem., 3686-3688 (2001) and Glass and Rosenfeld, 14 Genes & Development, 121-141 (2000). Preferred co-repressors useful in the methods of the present invention include SMRT (SEQ. ID. No. 14) and N-CoR (SEQ. ID. No. 15).

Co-repressors can include fragments of the above co-factors as well as the full-length protein. Such fragments will typically comprise at least one interaction domain ((LXXI/HIXXXI/L (SEQ. ID. No. 10) and about 5 to 20 amino acids, derived from a full length co-repressor sequence, immediately N-terminal of the interaction domain, and about 5 to 10 amino acids, derived from the co-repressor sequence, immediately C-terminal of the interaction domain. In some embodiments the fragment may contain two interaction domains, or in some embodiments at least three interaction domains. Preferred fragments of SMRT (SEQ. ID. No. 14) include amino acids 2131-2352 of the coding sequence, while preferred fragments of NcoR (SEQ. ID. No. 15) include amino acids 794-1397 of the coding sequence.

Alternatively corepressors can include amino acid sequences which are not found in nature but which are identified by a peptide screening method as binding to a LBD of a nuclear receptor in an antagonist-dependent manner. Typically such sequences will exhibit substantial identity to the corresponding sequences of known corepressors.

Preferred co-repressors of this type include, without limitation those listed in Table 4.

TABLE 4

| Co-repressor Peptide | |
|---|---|
| Sequence | Sequence ID listing |
| RLITLADHICQIITQDFAR | SEQ. ID. No. 43 |
| ASNLGLEDIIRKALMG | SEQ. ID. No. 44 |
| RVVTLAQHISEVITQDYTR | SEQ. ID. No. 45 |
| ASTMGLEAIIRKALMG | SEQ. ID. No. 86 |

Transactivation domains are well known in the art and can be readily identified by the artisan. Examples include the GAL4 activation domain (SEQ. ID. No. 21), TAT (SEQ. ID. No. 20), VP16 (SEQ. ID. No. 19), and analogs thereof.

Numerous methods of co-transfecting the expression and reporter plasmids are known to those of skill in the art and may be used for the co-transfection assay to introduce the plasmids into a suitable cell type.

In another embodiment of this method, two modified host cells are used, in which each modified host cell comprises either a co-activator recruitment assay system, or a co-repressor recruitment system. This approach is easier to set up and requires less molecular genetic manipulation than the method above.

Accordingly, in one embodiment, the present invention includes a method to identify compounds that bind to a nuclear receptor and exhibit cell type specific actions, which comprises:

a) contacting a first and second modified host cell with a test compound, wherein said first modified host cell comprises:

i) a first fusion protein, comprising a co-activator fused to a first heterologous DNA binding domain,

ii) a second fusion protein comprising a ligand binding domain of a nuclear receptor of interest fused to a first transcription activation domain,

iii) a first reporter gene operably linked to a first transcriptional regulatory sequence specific for said first heterologous DNA binding domain, and

wherein said second modified host cell comprises,

i) a third fusion protein, comprising a co-repressor fused to said first heterologous DNA binding domain or a second heterologous DNA binding domain,

ii) a fourth fusion protein comprising said ligand binding domain of the nuclear receptor of interest (“prey”) fused to said first transcription activation domain or a second transcription activation domain,

iii) a second reporter gene operably linked to said first transcriptional regulatory sequence specific for said first heterologous DNA binding domain or a second transcriptional regulatory sequence specific for said second heterologous DNA binding domain,

b) identifying those test compounds which cause altered expression of said first reporter gene product in said first modified host cell compared to a first modified host control cell, and similar or altered expression of said second reporter gene product in said second modified host cell, compared to a second modified host control cell.

In one embodiment of this method, the method further comprises the step of prescreening the test compounds to determine their affinity for the nuclear receptor of interest. In one aspect the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

In one embodiment of this method the first modified host cell and second modified host cell comprise separate reporter genes, which can be independently measured after the cells are mixed together.

In one embodiment of this method, the method further comprises the step of adding the test compound in the presence of a known antagonist, and identifying those compounds which caused reduced expression of said second reporter gene product without increasing expression of said first reporter gene.

In one embodiment of this method, the method further comprises the step of adding the test compound in the presence of a known agonist, and identifying those compounds which caused reduced expression of said first reporter gene product without increasing expression of said second reporter gene.

In another embodiment, the first and second modified host cells contain the same reporter gene, and are spatially separated, for example by being placed in separate wells of a 96 well multiwell plate, in order to generate independent readouts of co-activator and co-repressor interactions.

It is possible to use the above methods to identify compounds that interfere with particular protein-protein interactions, for example to identify compounds that provide for co-repressor dissociation. However, in the two-hybrid assays described above, such interference will result in a negative signal, i.e. failure to obtain expression of the reporter gene, which can lead to poor assay sensitivity and create a high false positive hit rate due to compound toxicity. Thus, these methods are well suited for identifying a positive interaction of polypeptide sequences, but are less suited for identifying test compounds which cause the dissociation of protein-protein interactions.

A method that overcomes this limitation is the “Reverse Two-Hybrid” approach, (see for example, Erickson et al. U.S. Pat. No. 5,525,490, Vidal et al. International Application Number PCT/US96/04995.

Accordingly in one aspect of the claimed invention, a standard two-hybrid assay is multiplexed with a reverse two-hybrid assay to provide for high sensitivity detection of both co-factor recruitment and dissociation.

Accordingly in one embodiment, the present invention includes methods using a reverse two hybrid assay to identify compounds that bind to a nuclear receptor and exhibit cell type specific actions, said method comprising:

a) contacting a modified host cell with a test compound, wherein said modified host cell comprises:

i) a first fusion protein, comprising a co-activator, fused to a first heterologous DNA binding domain,

ii) a second fusion protein comprising a co-repressor fused to a second heterologous DNA binding domain,

iii) a third fusion protein comprising a ligand binding domain of the nuclear receptor of interest fused to a transcription activation domain,

iv) a first reporter gene operably linked to a first transcriptional regulatory sequence specific for said first heterologous DNA binding domain,

v) a relay protein operably linked to a second transcriptional regulatory sequence specific for said second heterologous DNA binding domain,

vi) a second reporter gene operably linked to a third transcriptional regulatory sequence that is repressed by expression of said relay protein,

b) identifying those test compounds which cause altered expression of said first reporter gene product and similar, or altered expression of said second reporter gene product compared to a control modified host cell.

In one embodiment of this method, the method further comprises the step of prescreening the test compounds to determine their affinity for the nuclear receptor of interest. In one aspect the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

In one embodiment of this method, the method further comprises the step of adding the test compound in the presence of a known antagonist, and identifying those compounds which caused increased expression of said second reporter gene product without increasing expression of said first reporter gene.

In one embodiment of this method, the method further comprises the step of adding the test compound in the presence of a known agonist, and identifying those compounds which caused increased or no change in the expression of said second reporter gene product without increasing expression of said first reporter gene.

In another embodiment, this method comprises the use of two modified host cells, in which each modified host cell comprises either a co-activator recruitment assay system, or a co-repressor recruitment system. In a preferred embodiment of this method the co-activator recruitment assay is coupled to a positive two-hybrid assay and the co-repressor recruitment assay is coupled to a reverse two-hybrid assay. In some applications, and for re-testing, the alternative arrangement may also be preferred, i.e. co-repressor recruitment is coupled to a positive two-hybrid assay, and co-activator recruitment is coupled to a reverse two-hybrid assay.

Accordingly in one aspect, the present invention comprises a method to identify compounds that bind to a nuclear receptor and exhibit cell type specific actions, comprising:

a) contacting a first and second modified host cell with a test compound, wherein said first modified host cell comprises:

i) a first fusion protein, comprising a co-activator fused to a first heterologous DNA binding domain,

ii) a second fusion protein comprising a ligand binding domain of a nuclear receptor of interest fused to a first transcription activation domain,

iii) a first reporter gene operably linked to a first transcriptional regulatory sequence specific for said first heterologous DNA binding domain, and

wherein said second modified host cell comprises:

v) a third fusion protein, comprising a co-repressor fused to said first heterologous binding domain or a second heterologous binding domain,

vi) a fourth fusion protein, comprising said ligand binding domain of the nuclear receptor of interest fused to said first transcription activation domain or a second transcription activation domain,

vii) a relay plasmid comprising DNA encoding a relay protein operably linked to said first transcriptional regulatory sequence specific for said first heterologous DNA binding domain or to said second transcriptional regulatory sequence specific for said second heterologous DNA binding domain,

viii) a second reporter gene operably linked to a third transcriptional regulatory sequence that is repressed by expression of said relay protein,

b) identifying those test compounds which cause altered expression of said first reporter gene product in said first modified host cell compared to a first modified host control cell, and similar or altered expression of said second reporter gene product in said second modified host cell, compared to a second modified host control cell.

In some embodiments of the method, the first reporter gene and the second reporter gene provide two independent readouts. In one embodiment of this method the modified host cells can comprise separate reporter genes, which can be independently measured after the cells are mixed together.

In one embodiment of this method, the method further comprises the step of adding the test compound in the presence of a known antagonist, and identifying those compounds which caused increased expression of said second reporter gene product without increasing expression of said first reporter gene.

In one embodiment of this method, the method further comprises the step of adding the test compound in the presence of a known agonist, and identifying those compounds which caused increased or no change in the expression of said second reporter gene product without increasing expression of said first reporter gene.

Alternatively if the two modified host cells contain the same reporter gene, they may be spatially separated, for example by being placed in separate wells of a 96 well multiwell plate, in order to generate independent readouts of co-activator and co-repressor interactions.

In one embodiment of this method, the method further comprises the step of prescreening the test compounds to determine their affinity for the nuclear receptor of interest. In one aspect the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

In one embodiment of the reverse two-hybrid system, the relay binds to and blocks the activation domain(s) of transcriptional activators. Only when the level of the masking protein is reduced because a compound interferes with the two-hybrid interaction will the activation domain of the transcriptional activator be unmasked and allowed to function.

Although a variety of suitable relay proteins are apparent to those of skill in the art, this category of relay protein can be exemplified by the mammalian mdm2 oncoprotein (Accession No. NM_{—}006882) which binds to the transactivation domain of the tumor suppressor protein p53 (Accession No AH007667), and the yeast Gal80 protein (Accession No X01667) which binds and inactivates the activation domain of Gal4 (SEQ. ID. No. 21).

In another embodiment, the relay protein comprises a mutation, addition, or deletion that reduces the stability of the relay protein in vivo as compared to the naturally occurring cognate relay protein.

In another embodiment of the reverse two hybrid assay a transcriptional repressor can be used in place of the transcriptional activator in the traditional two-hybrid assay. See for example Sadowski, et al. U.S. Pat. No. 5,885,779. In this system, interaction between a ‘bait’ fusion protein having a DNA-binding domain, such as the DNA-binding domain of GAL4 (SEQ. ID. No. 17) or LexA (SEQ. ID. No. 18) with a ‘prey’ fusion protein having a repression domain, such as the N-terminal TUP1 repression domain (Accession No. U92792) causes inhibition of expression, i.e. repression, of specific reporter genes.

In another aspect the invention includes a composition comprising,

a) a modified host cell which comprises:

i) a first fusion protein, comprising a co-activator fused to a first heterologous DNA binding domain,

ii) a second fusion protein comprising a co-repressor fused to a second heterologous DNA binding domain,

iii) a third fusion protein comprising a ligand binding domain of the nuclear receptor of interest fused to a transcription activation domain,

iv) a first reporter gene operably linked to a first transcriptional regulatory sequence specific for said first heterologous DNA binding domain,

v) a relay protein operably linked to a second transcriptional regulatory sequence specific for said second heterologous DNA binding domain,

vi) a second reporter gene operably linked to a third transcriptional regulatory sequence that is repressed by expression of said relay protein,

b) a test compound.

In one aspect of the invention the test compound has a Kd for the nuclear receptor of interest, as measured by any of the methods disclosed herein, of at least 500 nM. In another aspect the test compound has a Kd for the nuclear receptor of interest, as measured by any of the methods disclosed herein, of at least 200 nM. In another aspect the test compound has a Kd for the nuclear receptor of interest, as measured by any of the methods disclosed herein, of at least 100 nM.

In yet another aspect the invention comprises a composition comprising,

a modified host cell which comprises:

i) a first fusion, comprising the co-activator fused to a first heterologous DNA binding domain,

ii) a second fusion protein comprising the co-repressor fused to a second heterologous DNA binding domain,

iii) a third fusion protein comprising the ligand binding domain of the nuclear receptor of interest fused to a transcription activation domain,

iv) a first reporter gene operably linked to a first transcriptional regulatory sequence specific for said first heterologous DNA binding domain,

v) a second reporter gene operably linked to a second transcriptional regulatory sequence specific for said second heterologous DNA binding domain,

vi) a test compound.

In one aspect of the invention the test compound has a Kd for the nuclear receptor of interest, as measured by any of the methods disclosed herein, of at least 500 nM. In another aspect the test compound has a Kd for the nuclear receptor of interest, as measured by any of the methods disclosed herein, of at least 200 nM. In another aspect the test compound has a Kd for the nuclear receptor of interest, as measured by any of the methods disclosed herein, of at least 100 nM.

A variety of biochemical assay formats can also be used to determine co-factor recruitment. Example methods include, but are not limited to, gel shift assays (See, e.g., Forman et al., in Cell 81:687-693 (1995)), immunological/affinity methods (See, e.g., Yao et al., in Nature 366:476-479 (1993)), surface plasmon resonance (See, e.g., Fisher and Fivash in Curr. opin. Biotechnol. 5:389-395 (1994)), circular dichroism and optical rotary dispersion (See, e.g., Toney et al., in Biochemistry 32:2-6 (1993)), fluorescence anisotropy (See, e.g., Kersten et al., in Biochemistry 34:13717-13721 (1995)), nuclear magnetic resonance (See, e.g., Jenkins in Life Sciences 48:1227-1240 (1991)), and the like. Thus, those of skill in the art will readily recognize that the contacting contemplated by the above-described methods can be carried out in solution, or in the solid phase.

In some embodiments, standard gel shift assays are performed to determine the effects of test compounds on the binding of a co-activator or co-repressor to a LXR heterodimer/DNA complex. Reaction products are analyzed on a denaturing polyacrylamide gel. Kits for performing gel shift assays include for example, Gel Shift Assay Systems (Promega, Madison, Wis.). Assays that are readily amenable to multiplexed analysis, parallel processing and high throughput screening are preferred.

In one variation of the invention, direct physical interaction, measured as binding, between a LBD domain and a co-activator or co-repressor domain as a consequence of a test compound can be determined. In one aspect, an LBD domain is immobilized on a capture surface and a soluble, labeled or epitope-tagged co-activator or co-repressor domain is introduced under aqueous physiological conditions, either in the absence or presence of a known ligand or a test agent. A typical format of the direct method can be an ELISA, for illustration. Agents which produce a ligand-induced binding between the immobilized LBD and the soluble, labeled or epitope-tagged co-activator or co-repressor domain, result in the co-activator or co-repressor becoming immobilized on the capture surface enabling the identification candidate compounds.

In a variation, the co-activator or co-repressor domain can be immobilized on the capture surface and the LBD can be labeled or epitope-tagged. Epitope-tagged proteins can generally be detected by immunochemical methods using at least one antibody species that is specifically reactive with the epitope.

In another variation, the LBD species (or a multiplicity thereof) is immobilized on the capture surface and the soluble, labeled co-activator and co-repressor species (or a multiplicity of species thereof) can be used to identify ligand-induced binding interactions or ligand-dependent relief of binding interactions between an LBD and the co-activator and co-repressor domains simultaneously.

Vice versa, a co-activator or co-repressor domain (or multiple species thereof) can be immobilized on the capture surface and multiple species of uniquely labeled or tagged LBDs may be used. In each case, a test agent can be evaluated for its ability to produce a concentration-dependent binding between LBD and the co-activator or co-repressor species and compared to a parallel reaction lacking agent and/or to a parallel reaction lacking agent and containing a known ligand, either agonist or antagonist.

In such variations, it is usually preferable to employ distinctive labels or epitope-tags for each species of co-activator and co-repressor, which can provide a basis for the discrimination of co-activator and co-repressor binding to the LBD species (or a collection of LBD species) based upon unique detection of each label or tag on the capture surface.

For multiplexed analysis Europium (Eu) in combination with Samarium (Sm) or Terbium (Tb) can be used. Europium (Eu) gives high fluorescence and has the best sensitivity for use for detection of the analyte that requires higher sensitivity. Typically Samarium (Sm) or Terbium (Tb) can be used as the second label for measuring the analyte of lower sensitivity. (Noris et al. Science 1999 285 744-6; J. Immunol Methods 1996, 190, 171-83; J. Aric. Food. Chem. 2000 48 5868-73).

Accordingly in one aspect, the present invention includes a method to identify compounds that bind to a nuclear receptor and exhibit cell type specific actions said method comprising:

a) providing a composition comprising,

i) an affinity support, comprising a first fusion protein comprising a ligand binding domain of the nuclear receptor of interest fused to an affinity tag that couples said first fusion protein to said affinity support,

ii) a second fusion protein, comprising a co-activator coupled to a first detectable label,

iii) a third fusion protein comprising a co-repressor coupled to a second detectable label,

b) incubating said composition in an aqueous buffer comprising a test compound,

c) detecting the binding of said co-activator and said co-repressor to said first fusion protein,

d) identifying those test compounds which cause altered binding of said co-repressor and similar or altered binding of said co-activator to said ligand binding domain compared to a control composition.

In one embodiment of this method, test compounds are selected that cause disrupted, or substantially disrupted binding of said co-repressor without increasing binding of said co-activator to said ligand binding domain compared to a control composition.

In another embodiment of this method, the method further comprises the step of adding the test compound in the presence of a known antagonist, and identifying those compounds which caused disrupted, or substantially disrupted binding of said co-repressor without increasing binding of said co-activator.

In another embodiment of this method, the method further comprises the step of adding the test compound in the presence of a known agonist, and identifying those compounds which caused disrupted, or substantially disrupted binding of said co-activator without increasing binding of said co repressor.

In another embodiment of this method, the method further comprises the step of prescreening the test compounds to determine their affinity for the nuclear receptor of interest. In one aspect, the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

In another embodiment, the present invention includes a composition comprising,

i) an affinity support, comprising a first fusion protein comprising a ligand binding domain of the nuclear receptor of interest fused to an affinity tag that couples said first fusion protein to said affinity support,

ii) a second fusion protein, comprising a co-activator coupled to a first detectable label,

iii) a third fusion protein comprising a co-repressor coupled to a second detectable label,

iv) a test compound.

In another embodiment of this composition, the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

In another embodiment, the present invention includes a method to identify compounds that bind to a nuclear receptor and exhibit cell type specific actions, said method comprising:

a) providing first and second compositions, wherein said first composition comprises;

i) a ligand binding domain of a nuclear receptor of interest, and

ii) a co-activator coupled to a detectable label, and

wherein said second composition comprises;

iii) said ligand binding domain, and

v) a co-repressor coupled to said detectable label,

b) incubating said first composition and said second composition in an aqueous buffer comprising a test compound,

c) detecting the binding of said co-activator with said ligand binding domain in said first composition and detecting the binding of said co-repressor with said ligand binding domain in said second composition,

d) identifying those test compounds which cause altered binding of said co-repressor and similar or altered binding of said co-activator to said ligand binding domain compared to a control composition.

In one embodiment of this method, test compounds are selected that cause disrupted, or substantially disrupted binding of said co-repressor without increasing binding of said co-activator to said ligand binding domain compared to a control composition.

In one embodiment of this method, the method further comprises the step of adding the test compound in the presence of a known antagonist, and identifying those compounds which caused disrupted or substantially disrupted binding of said co-repressor without increasing binding of said co-activator.

In one embodiment of this method, the method further comprises the step of adding the test compound in the presence of a known agonist, and identifying those compounds which caused disrupted or substantially disrupted binding of said co-activator without increasing binding of said co repressor.

In another embodiment of this method, the method further comprises the step of prescreening the test compounds to determine their affinity for the nuclear receptor of interest. In one aspect, the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

In another embodiment, the present invention includes a method to identify compounds that bind to a nuclear receptor and exhibit cell type specific actions, said method comprising:

a) providing first and second compositions, wherein said first composition comprises;

i) a ligand binding domain of a nuclear receptor of interest, coupled to a first detectable label, and

ii) a co-activator coupled to a second detectable label, and

wherein said second composition comprises;

iii) said ligand binding domain, coupled to said first detectable label, and

iv) a co-repressor coupled to said second detectable label,

b) incubating said first composition and said second composition in an aqueous buffer comprising a test compound,

c) detecting the binding of said co-activator with said ligand binding domain in said first composition and detecting the binding of said co-repressor with said ligand binding domain in said second composition,

d) identifying those test compounds which cause altered binding of said co-repressor and similar or altered binding of said co-activator to said ligand binding domain compared to a control composition

In one embodiment of this method, test compounds are selected that cause disrupted, or substantially disrupted binding of said co-repressor without increasing binding of said co-activator to said ligand binding domain compared to a control composition.

In one embodiment of this method, the method further comprises the step of adding the test compound in the presence of a known antagonist, and identifying those compounds which caused disrupted or substantially disrupted binding of said co-repressor without increasing binding of said co-activator.

In one embodiment of this method, the method further comprises the step of adding the test compound in the presence of a known agonist, and identifying those compounds which caused disrupted or substantially disrupted binding of said co-activator without increasing binding of said co repressor.

In another embodiment of this method, the method further comprises the step of prescreening the test compounds to determine their affinity for the nuclear receptor of interest. In one aspect, the test compounds have a Kd for the nuclear receptor of interest, as measured by any of the methods disclosed herein, of at least 1000 nM. In another aspect the test compounds have a Kd for the nuclear receptor of interest, as measured by any of the methods disclosed herein, of at least 200 nM. In another aspect the test compounds have a Kd for the nuclear receptor of interest, as measured by any of the methods disclosed herein, of at least 100 nM.

### Whole Animal Studies

Additionally the compounds and compositions can be evaluated for their ability to increase or decrease the expression of genes known to be modulated by LXR α or β and other nuclear receptors in vivo, using Northern-blot, RT PCR or oligonucleotide microarray analysis to analyze RNA levels. Western-blot analysis can be used to measure expression of proteins encoded by LXR target genes. Genes that are known to be regulated by the LXRs include the ATP binding cassette transporters ABCA1, ABCG1, ABCG5, ABCG8, the sterol response element binding protein 1c (SREBP1c) gene, stearoyl CoA desaturase 1 (SCD-1) and the apolipoprotein apoE gene (ApoE).

Established animal models exist for a number of diseases of direct relevance to the claimed compounds and these can be used to further profile and characterize the claimed compounds. These model systems include diabetic dislipidemia using Zucker (fa/fa) rats or (db/db) mice, spontaneous hyperlipidemia using apolipoprotein E deficient mice (ApoE^{-/-}), diet-induced hyperlipidemia, using low density lipoprotein receptor deficient mice (LDR^{-/-}) and atherosclerosis using both the Apo E(^{-/-}) and LDL(^{-/-}) mice fed a western diet. (21% fat, 0.05% cholesterol). Additionally LXR or FXR animal models (e.g., knockout mice) can be used to further evaluate the present compounds and compositions in vivo (see, for example, Peet, et al., Cell (1998), Vol. 93, pp. 693-704, and Sinal, et al., Cell (2000), Vol. 102, pp. 731-744).

### Therapeutic Applications

Disorders of lipoprotein metabolism and the susceptibility of human metabolism to adverse effects from diets high is saturated fats have resulted in epidemic atherosclerotic disease in the United States and other developed countries.

One aspect of lipid metabolism, and a primary figure in atherosclerotic disease is cholesterol. Lipoproteins transport cholesterol and triglycerides, which are not water soluble, from sites of absorption and synthesis to sites of utilization. Lipoproteins are classified into six major groups based on size, density, electrophoretic mobility and lipid/protein composition. These six classes are chylomicrons (dietary triglycerides),VLDL (endogenous triglycerides), IDL (cholesterol ester, triglycerides), LDL (cholesterol ester), HDL (cholesterol ester), and Lp (cholesterol ester) with the major lipid in each group represented parenthetically. There are both exogenous (dietary) and endogenous (primarily liver) sources of cholesterol. Similarly, there is an exogenous lipid transport pathway to transport dietary cholesterol absorbed in the intestine, and an endogenous pathway to transport cholesterol secreted by the liver.

There are several clinical manifestations associated with lipoprotein disorders due to a breakdown somewhere along the route of lipid metabolism which result in elevated cholesterol levels. The present invention seeks to reduce LDL cholesterol levels while increasing HDL cholesterol levels by administering therapeutic agents as described herein. Generally, the relevant disease states are those where in vivo cholesterol levels are above the desired cut off for the particular clinical situation of the patient. Obviously, the level can vary depending upon the age, gender, genetic background and health of the patient.

Examples of disease suitable for treatment according to the present invention are familial lipoprotein lipase deficiency, an autosomal recessive disorder; familial apolipoprotein C-11 deficiency, an autosomal recessive disorder; familial hypertriglyceridemia, an autosomal dominant disorder; familial defective apolipoprotein B-100; and familial combined hyperlipidemia. All of these lipid-related diseases can lead to elevated cholesterol levels. However, the most common cause of high blood cholesterol is due to familial hypercholesterolemia (FH). FH is an autosomal dominant disorder caused by a mutation in the gene encoding the LDL receptor protein. Five classes of mutant alleles have been identified that cause a functional or absolute LDL receptor deficiency. Treatment of all of these diseases is contemplated as part of the present invention.

Therapies also may encompass diagnostic procedures to establish the need for a particular therapeutic regimen. Methods for determining cholesterol levels are well known, widely practiced, and many commercial kits are readily available. It also is envisioned that continued monitoring of cholesterol levels throughout a course of treatment will be utilized, both to assess the efficacy of the treatment, and to establish whether an increase or decrease in the drug dosage is required.

In certain embodiments, the combined administration of cholesterol adsorption inhibitors with other drugs can prove particularly advantageous. Other compounds that may be used in conjunction with modulators of the present invention are (a) PPAR agonists and partial agonists, or (b) one or more of the three classes of antilipernic agents currently in use for treating lipid disorders, or both (a) and (b). The three groups of antilipernic drugs are classified as (i) bile acid sequestrants, (ii) fibric acid derivatives and (iii) HMG-CoA reductase inhibitors.

Once isolated, a modulator or analog thereof can be put in pharmaceutically acceptable formulations, such as those described in Remington's Pharmaceutical Sciences, 18th ed., Mack Publishing Co., Easton, Pa. (1990), incorporated by reference herein, and used for specific treatment of diseases and pathological conditions with little or no effect on healthy tissues.

In a preferred embodiment, the composition is held within a container which includes a label stating to the effect that the composition is approved by the FDA in the United States (or other equivalent labels in other countries) for treating a disease or condition described herein. Such a container will provide therapeutically effective amount of the active ingredient to be administered to a host.

The particular modulators that affects the disorders or conditions of interest can be administered to a patient either by themselves, or in pharmaceutical compositions where it is mixed with suitable carriers or excipient(s). In treating a patient exhibiting a disorder of interest, a therapeutically effective amount of a agent or agents such as these is administered. A therapeutically effective dose refers to that amount of the compound that results in amelioration of symptoms or a prolongation of survival in a patient.

The compounds also can be prepared as pharmaceutically acceptable salts. Examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. (See e.g., PCT/US92/03736). Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate administration of the compound, for example, to increase the solubility of the compound. Examples of carriers and excipients include calcium carbonate, calcium phosphate, various sugars or types of starch, cellulose derivatives, gelatin, vegetable oils, polyethylene glycols and physiologically compatible solvents. In addition, the molecules tested can be used to determine the structural features that enable them to act on the ob gene control region, and thus to select molecules useful in this invention. Those skilled in the art will know how to design drugs from lead molecules, using techniques such as those disclosed in PCT publication WO 94/18959, incorporated by reference herein.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Some methods of delivery that may be used include:

a. encapsulation in liposomes,

b. transduction by retroviral vectors,

c. localization to nuclear compartment utilizing nuclear targeting site found on most nuclear proteins,

d. transfection of cells ex vivo with subsequent re-implantation or administration of the transfected cells,

e. a DNA transporter system.

All publications referenced are incorporated by reference herein, including the nucleic acid sequences and amino acid sequences listed in each publication. All the compounds disclosed and referred to in the publications mentioned above are incorporated by reference herein, including those compounds disclosed and referred to in articles cited by the publications mentioned above.

While the invention has been described in detail with reference to certain preferred embodiments thereof, it will be understood that modifications and variations are within the spirit and scope of that which is described and claimed.

### EXAMPLES

General Methods

RNA isolation and analysis of gene expression by quantitative RT-PCR. Total RNA from mouse tissues and cells was isolated using RNeasy kits (QIAGEN Inc.) according to the supplier's total RNA isolation procedure. Real time PCR was performed using a Perkin-Elmer/ABI 7700 Prism. RNA samples were DNase treated with 1 unit RNase free, DNase (Roche) per 1.6 μg total RNA for 40 minutes at 37° C. followed by a 10 minute incubation at 75° C. For each target quadruplicate reactions each containing 100 ng of total RNA (including one minus reverse transcriptase control) were utilized. RNA was reverse transcribed using 10 units of Superscript II reverse transcriptase (Life Technologies), 400 nM of a target specific reverse primer, 500 uM dNTPs, 10 mM DTT and 1× Superscript II buffer. Quantitative PCR of reverse transcriptase reactions was carried out with 1.25 units Taq polymerase (Life Technologies), 1× Taq buffer, 3 mM MgCl₂, 200 uM dNTPs, 400 nM target specific forward and reverse primers and 100 nM target specific fluorogenic probe. All assays were run for 40 cycles (95° C. for 12 seconds followed by 60° C. for 60 seconds). Probes and primers were designed using Primer Express (ABI). Levels of cyclophilin were measured in all in vivo samples and the results are presented as number of target transcripts per cyclophilin transcript.

Animals. Mice deficient in both LXRα (SEQ. ID. No. 6) and LXRβ (SEQ. ID. No. 7) (LXRαβ^{-/-}) were generated in a mixed genetic background (C57BL/6x A129). The appropriate strain matched wild type control was used for all experiments.

HDL measurements. T0901317 (N-(2,2,2,-trifluoro-ethyl)-N-[4-(2,2,2-trifluoro-1-hydroxy-1-trifluoromethyl-ethyl)-phenyl]-benzenesulfonamide)(X-Ceptor Therapeutics, Inc., San Diego, Calif.) was administered by daily oral gavage for seven days in polyethylene glycol/tween 80 vehicle via a 1-cc syringe fitted with a disposable feeding needle. Compound was solvated in ethanol (5% final volume) and brought up to final volume in vehicle. On day seven mice were anesthetized with isofluorane and blood samples were obtained by retro-orbital plexus puncture. Blood samples were obtained in heparinized tubes, centrifuged to obtain plasma and stored at −20° C. HDL levels were determined by precipitating non-HDL cholesterol from plasma using a precipitating reagent (Wako Diagnostic 278-64709, Richmond Va.). The remaining HDL cholesterol was quantitated using a colorimetric enzymatic assay adapted to a 96 well plate (Infinity Total Cholesterol Reagent, Sigma, St. Louis, Mo.).

Cholesterol efflux. Peritoneal macrophage isolated from wild type and LXRαβ^{-/-} mice were labeled with ¹⁴C-cholesterol for an additional 48 hours. Labeled cells were washed, and efflux was initiated in medium with or without 10 μg/ml apoAI in the absence or presence of T0901317. After 24 hours, media was removed, cell debris was pelleted, and radioactivity in the media was determined by scintillation counting. To determine the cell associated radioactivity, cells were lysed in 0.2 M sodium hydroxide and radioactivity was determine by scintillation counting. Percent efflux was calculated by dividing the radioactivity in the media by the sum of the radioactivity in the media and cell lysate. ApoAI-dependent efflux was determined by subtracting the efflux observed in the absence of added apoAI.

GAL4 one-hybrid experiments. CV-1 cells were transfected using FuGene6 (Roche Applied Science, Indianapolis, Ind.) per manufacturer's instructions in 96 well plates with a total of 65 ng of DNA per well, consisting of 20 ng β-gal reporter, 15 ng GAL4-UAS4xRE (SEQ. ID. No. 28), 15 ng GAL4-LXR full length, and 15 ng pCMX (Cell Jun. 28, 1991; 65(7):1255-66). Media containing ligand was added directly to the cells 5 hours after transfection. Cells were harvested 18 hours later and analyzed for luciferase and β-gal activity. Luciferase activity is normalized to β-gal activity. The GAL4-LXR constructs used in these assays encode amino acids 1 to 147 of Gal4 (SEQ. ID. No. 17) fused in frame to the N-terminus of full length human LXRα (SEQ. ID. No. 6) or LXRβ (SEQ. ID. No. 7).

GAL4 two-hybrid experiments. CV-1 cells were transfected using FuGene6 (Roche Applied Science, Indianapolis, Ind.) per manufacturer's instructions in 96 well plates with a total of 65 ng of DNA per well, consisting of 20 ng β-gal reporter, 15 ng GAL4-UAS4xRE (SEQ. ID. No. 28), 15 ng VP16-LXR-LBD, and 15 ng GAL4-human silencing mediator of retinoid and thyroid transcription (SMRT) receptor interacting domain 1 and 2 (ID1+ID2) or human nuclear receptor corepressor 1 (NCoR) ID1+ID2. Media containing ligand was added directly to the cells 5 hours after transfection. Cells were harvested 18 hours later and analyzed for luciferase and β-gal activity. Luciferase activity is normalized to β-gal activity. In the two-hybrid analysis the ligand binding domains of human LXRα (SEQ. ID. No. 6) (amino acids 164-447) or human LXRβ (SEQ. ID. No. 7) (amino acids 155-461) were fused to the VP16 activation domain (SEQ. ID. No. 19) and the receptor interacting domains of human SMRT (SEQ. ID. No. 14) (ID1+ID2, amino acids 2131-2352) and human NCoR (SEQ. ID. No. 15) (ID1+ID2, amino acids 794-1397) were fused to the GAL4 DNA binding domain (SEQ. ID. No. 17).

Identification of sequences that interact with LXR. Double stranded oligonucleotides encoding the 20 amino acids around the LXXLL (SEQ. ID. No. 9)interaction motifs derived from known nuclear receptor coactivators or chimeric 20 amino acids sequences derived from combining sequences from the interaction domains of human steroid receptor coactivator 1 (SRC-1) (SEQ. ID. No. 11) and mouse CREB binding protein (CBP) (SEQ. ID. No. 33) were fused in frame to the DNA binding domain of GAL4 (SEQ. ID. No. 17). CV-1 cells were transfected using FuGene6 (Roche Applied Science, Indianapolis, Ind.) per manufacturer's instructions in 96 well plates with a total of 65 ng of DNA per well, consisting of 20 ng β-gal reporter, 15 ng GAL4-UAS 4xRE (SEQ. ID. No. 28), 15 ng VP16-LXR-LBD, and 15 ng GAL4-interaction domain fusion protein. Media containing ligand was added directly to the cells 5 hours after transfection. Cells were harvested 18 hours later and analyzed for luciferase and β-gal activity. Luciferase activity is normalized to β-gal activity. In the two-hybrid analysis the ligand binding domain of human LXRα (SEQ. ID. No. 6) (amino acids 164-447) and human LXRβ (SEQ. ID. No. 7) (amino acids 155-461) were fused to the VP16 activation domain (SEQ. ID. No. 19).

Transient transfections in mouse embryonic fibroblasts. Mouse embryonic fibroblasts (MEFs) cells were transfected using FuGene6 (Roche Applied Science, Indianapolis, Ind.) per manufacturer's instructions in 48 well plates with a total of 150 ng DNA per well, consisting of 50 ng β-gal reporter, 50 ng GAL4-UAS 4xRE (SEQ. ID. No. 28), and 50 ng GAL4-LXR full length (see above). Media containing ligand was added directly to the cells 5 hours after transfection. Cells were harvested 18 hours later and analyzed for luciferase and β-gal activity. Luciferase activity is normalized to β-gal activity. The GAL4-LXR constructs used in these assays encode amino acids 1 to 147 of Gal4 (SEQ. ID. No. 17) fused in frame to the N-terminus of full length human LXRα (SEQ. ID. No. 6) or LXRβ (SEQ. ID. No. 7).

High Throughput Fret Coactivator Assay

High through FRET cofactor interaction assays were performed in 96 or 384 well assay well plates using automated liquid handling and analysis. Screens for LXR were performed using the protocol below. Equivalent screens for FXR were performed using the FXR (SEQ.ID. No. 4) ligand binding domain in place of LXRα and LXRβ LBD and at a concentration of 8 nM per well.

A. Required Materials:

1. Partially purified recombinant protein comprising glutathione-S-transferase fused in frame to the LXR-ligand binding domain (comprising amino acids 188-447 of human LXRα (SEQ. ID. No. 6), or amino acids 198-461 of human LXRβ (SEQ. ID. No. 7)).

2. Biotinylated peptide containing a SRC-1 receptor interaction motif(B-SRC-1) (SEQ. ID. No. 46).

3. Anti-GST antibody conjugated to an Europium chelate (αGST-K) (From Wallac/PE Life Sciences Cat# AD0064).

4. Streptavidin linked allophycocyanin (SA-APC) (From Wallac/PE Life Sciences CAT# AD0059A).

5. 1× FRET Buffer: (20 mM KH₂PO₄/K₂HPO₄ pH 7.3, 150 mM NaCl, 2.5 mM CHAPS, 2 mM EDTA, 1 mM DTT (add fresh)).

6. 96 well or 384 well black multiwell plates (from LJL).

Stock Solutions: 0.5 M KH₂PO₄/K₂HPO₄ (pH 7.3); 5 M NaCl; 80 mM (5%) CHAPS; 0.5 M EDTA (pH 8.0); 1 M DTT (store at −20° C.).

B. Preparation of Screening Reagents:

Reaction mixture was prepared for the appropriate number of wells by combining the following reagents: 5 nM/well GST-hLXR αLBD, 5 nM/well GST-hLXR βLBD, 5 nM/well Anti-GST antibody (Eu), 12 nM/well biotin-SRC-1 peptide, 12 nM/well APC-SA adjust the volume to 10 μL/well with 1×-FRET buffer.

C. Procedure:

a) 0.5 μL of a 1 mM stock test compound (for approx. 10 μM final concentration) or solvent was added to each well in a 96 well or 384 well black plate (LJL).

b) 10 μl reaction mixture (prepared above) was added to each well of the multiwell plate.

c) The samples were incubated covered in the dark at room temperature for 1-4 hours.

d) Plates were read using an LJL Analyst, or similar instrument, using the following conditions: Channel 1: The excitation wavelength was set to 330 nm, emitted light was collected at 615 nm, with 100 flashes per well, an integration time of 1000 μs; a 10 msec interval between flashes, and a delay after flashes of 200 μs. Channel 2: The excitation wavelength was set to 330 nm, and emitted light was collected at 665 nm, with 100 flashes per well, an integration time of 100 μs; a 10 msec interval between flashes, and a delay after flashes of 65 μs.

Co-Transfection Assay

High throughput co-transfection assays were performed in 96 or 384 well assay well plates using automated liquid handling and analysis. Screens were performed using the protocols below.

A Required Materials

1. CV-1 African Green Monkey Kidney Cells

2. Co-transfection Expression plasmids, CMX-hLXR, or CMX-hLXR, CMX-RXR, reporter (LXREx1-Tk-Luciferase), and control (CMX-Galactosidase expression vector) (see, Cell Jun. 28, 1991; 65(7):1255-66).

3. Transfection reagent such as FuGENE6 (Roche).

4. 1× Cell lysis buffer (1% Triton X 100 (JT Baker X200-07), 10% Glycerol (J T Baker M778-07), 5 mM Ditriotreitol (Quantum Bioprobe DTT03; add fresh before lysing), 1 mM EGTA (Ethylene Glycol-bis(B-Amino ethyl ether)-N,N,N′,N′-Tetracetic Acid) (Sigma E-4378), 25 mM Tricine (ICN 807420) pH 7.8)

5. 1× Luciferase assay buffer (pH at 7.8) (0.73 mM ATP, 22.3 mM Tricine, 0.11 mM EDTA, 33.3 mM DTT)

6. 1× Luciferrin/CoA (11 mM Luciferin, 3.05 mM Coenzyme A, 10 mM HEPES)

B. Preparation of Screening Reagents

CV-1 cells were prepared 24 hours prior to the experiment by plating them into T-175 flasks or 500 cm² dishes in order to achieve 70-80% confluency on the day of the transfection. The number of cells to be transfected was determined by the number of plates to be screened. Each 384 well plate requires 1.92×106 cells or 5000 cells per well.

DNA Transfection Reagent was prepared by mixing the required plasmid DNAs with a cationic lipid transfection reagent such as DOTAP or FuGENE6 by following the instructions provided with the reagents. Optimal DNA amounts were determined empirically per cell line and size of vessel to be transfected.

10-12 mL media was added to the DNA Transfection Reagent and this mixture was added to the cells after aspirating media from a T175 cm² flask. The plates were then incubated for at least 5 hours at 37° C. to prepare screening cells.

Luciferase assay reagent was prepared by combining before use (per 10 mL):

10 mL 1× Luciferase assay buffer

0.54 mL of 1× Luciferrin/CoA

0.54 mL of 0.2 M Magnesium sulfate

C. Procedure

a) Assay plates were prepared by dispensing 0.5 μL of 1 mM compound per well of a 384 well plate to achieve final compound concentration of 10 μM and 1% DMSO.

b) Media was removed from the screening cells, the cells trypsinized, harvested by centrifugation, counted and plated at 5000 cells per well in the 384 well assay plate (as prepared above in a volume of about 45 μL).

c) Assay plates were incubated with both compounds and screening cells for 20 hours at 37° C.

d) Media was carefully removed from cells, lysis buffer (30 μL/well) added and the plates left to incubate at least 30 minutes at room temperature.

e) After 30 minutes (30 μL/well luciferase assay buffer was added and the assay plates read immediately after buffer addition on luminometer (PE Biosystems Northstar reader with on-board injectors, or equivalent).

The LXR/LXRE co-transfection assay can be used to establish the EC₅₀/IC₅₀ values for potency and percent activity or inhibition for efficacy. Efficacy defines the activity of a compound relative to a high control ((N-(3-((4-fluorophenyl)-(naphthalene-2-sulfonyl)amino) propyl)-2,2-dimethylpropionamide)) or a low control (DMSO/vehicle). The dose response curves are generated from an 8 point curve with concentrations differing by ½ LOG units. Each point represents the average of 4 wells of data from a 384 well plate. The data from this assay is fitted to the following equation, from the EC₅₀ value may be solved:

Y=Bottom+(Top−Bottom)/(1+10^{((log EC50-X)*HillSlope)})

The EC₅₀/IC₅₀ is therefore defined as the concentration at which an agonist or antagonist elicits a response that is half way between the Top (maximum) and Bottom (baseline) values. The EC₅₀/IC₅₀ values represented are the averages of at least 3 independent experiments. The determination of the relative efficacy or % control for an agonist is by comparison to the maximum response achieved by ((N-(3-((4-fluorophenyl)-(naphthalene-2-sulfonyl)-amino)propyl)-2,2-dimethylpropionamide) that is measured individually in each dose response experiment.

For an antagonist assay, an agonist can be added to each well of a 384 well plate to elicit a response. The % inhibition for each antagonist is therefore a measurement of the inhibition of the activity of the agonist. In this example, 100% inhibition would indicate that the activity of a specific concentration of agonist that has been reduced to baseline levels, defined as the activity of the assay in the presence of DMSO only.

### Example 1

LXR Functions as Both an Activator and Repressor of ABCA1 and Cholesterol Efflux.

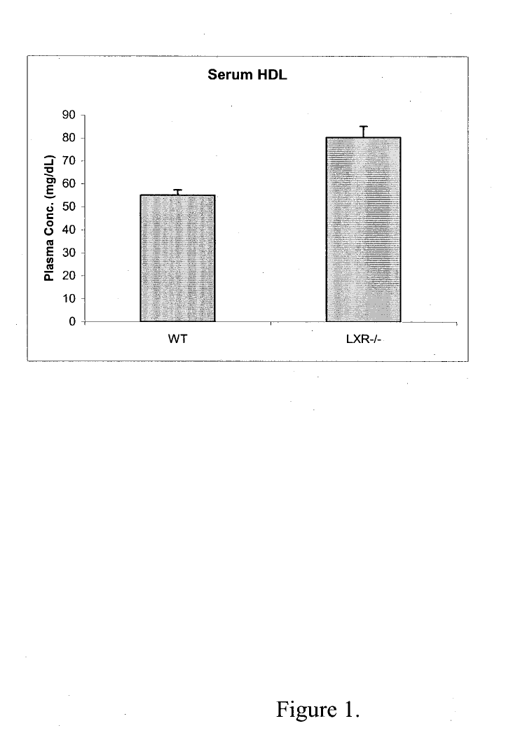

Analysis of serum HDL levels from wild type and LXRαβ^{-/-} mice maintained on a normal chow diet containing 0.02% cholesterol reveals that LXRαβ^{-/-} mice have significantly higher HDL levels (FIG. 1). LXR knockout mice were generated as described in Peet et al., 93 Cell, 693-704 (1998). Because serum HDL levels can be affected by the expression of the ATP binding cassette transporter ABCA1, we examined the effect of LXR on ABCA1 expression.

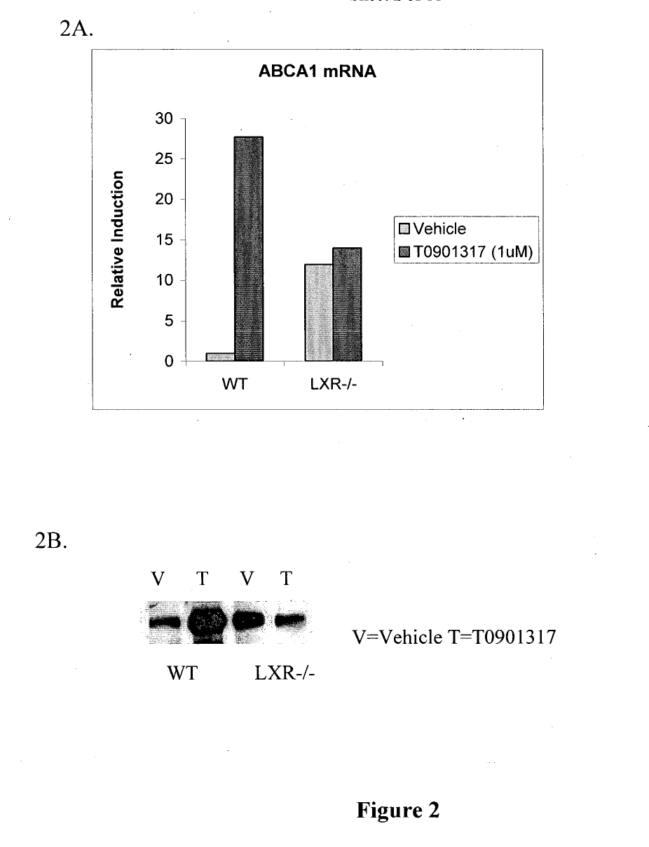

To examine the effects of LXR on ABCA1 regulation, peritoneal macrophage were isolated from wild type and LXR knockout mice. LXR knockout mice (LXRαβ^{-/-}) were generated as described in Peet et al., 93 Cell, 693-704 (1998). Macrophage were treated with vehicle or the LXR agonist T0901317 for 18 hours and examined for ABCA1 mRNA and protein levels. In FIG. 2A. RT-PCR was used to quantitate the levels of ABCA1 and the cyclophilin following induction of mRNA. ABCA1 levels were normalized to cyclophilin levels and the results are presented as fold induction above wild type macrophage treated with vehicle. In FIG. 2B. whole cells extracts were isolated, run on an SDS-PAGE and probed with an antibody specific for ABCA1 by western blot analysis.

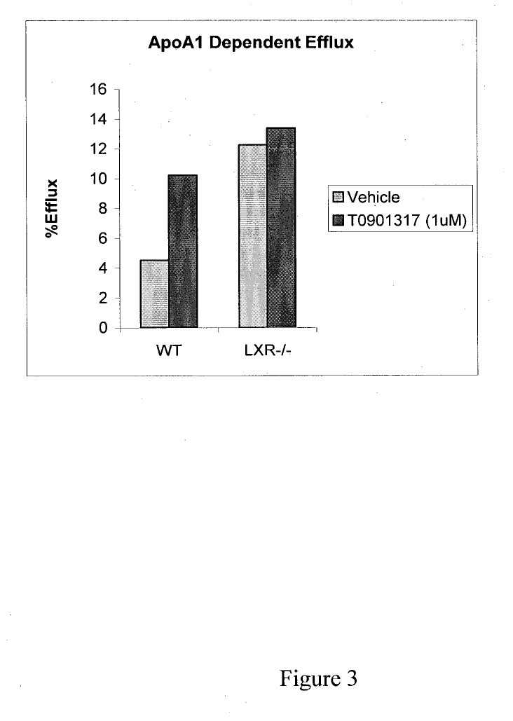

As expected treatment with T0901317 increases ABCA1 mRNA and protein levels in an LXR dependent manner. Loss of LXR also results in increases in ABCA1 mRNA and protein levels. To determine if loss of LXR also affects cholesterol efflux, peritoneal macrophage were labeled with [¹⁴C]-cholesterol to evaluate ApoA1 dependent efflux, see FIG. 3 In correlation with the increased ABCA1 levels, basal ApoA1 dependent efflux is greater in the LXRαβ^{-/-} macrophage compared to the wild type. Therefore, ABCA1 expression and cholesterol efflux can be increased by either ligand mediated activation of LXR or loss of LXR. This data demonstrate that ABCA1 expression is repressed by LXR in the absence of ligand.

### Example 2

LXR Repression is Gene Specific.

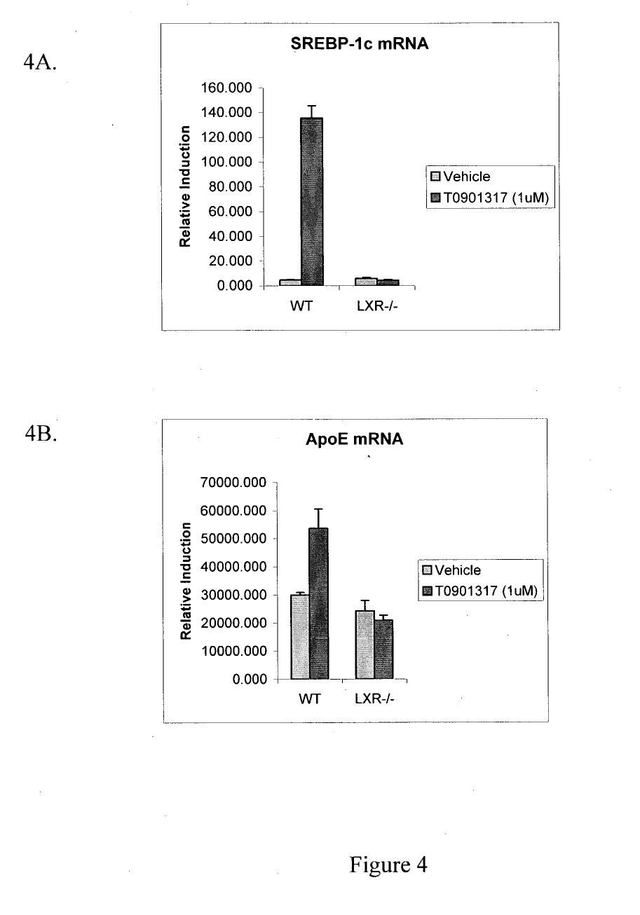

To determine if other LXR target genes are repressed by LXR in the absence of ligand we examined the mRNA levels of SREBP1c and ApoE in peritoneal macrophage from wild type and LXRαβ^{-/-} mice. Unlike ABCA1, SREBP1c and ApoE mRNA levels are not affected by loss of LXR, suggesting that the LXR mediated repression of ABCA1 is gene specific. See FIG. 4.

### Example 3

LXR Represses ABCA1 in a Tissue Specific Manner.

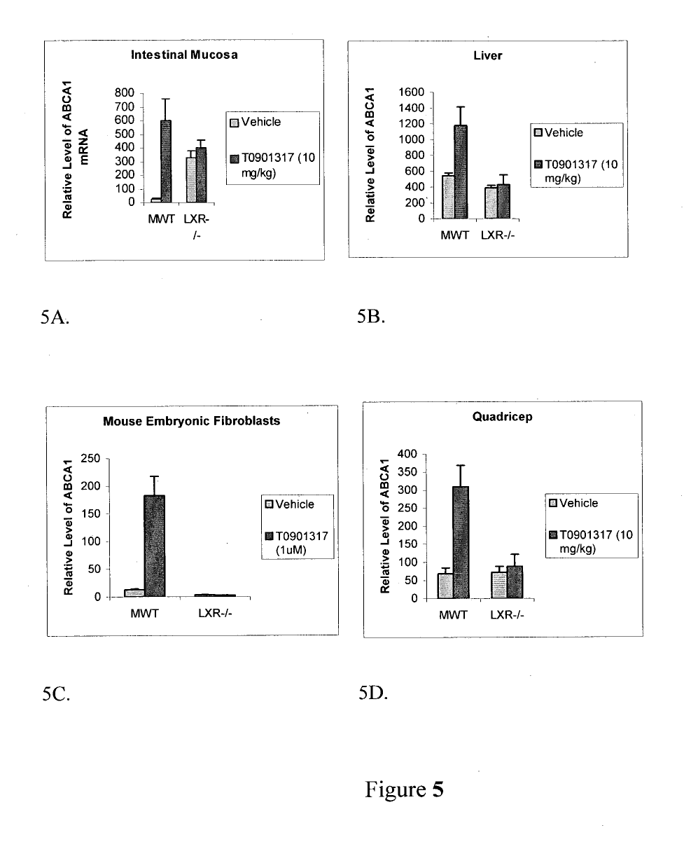

To further examine LXR regulation of ABCA1 in other tissues wild type and LXRαβ^{-/-} mice were dosed daily with 10 mg/kg T0901317 for 7 days. ABCA1 levels were also measured in isolated mouse embryonic fibroblasts treated with T091317 in culture. LXR agonist treatment increases expression of ABCA1 mRNA in all of the tissues analyzed, in an LXR dependent manner. Intestinal mucosa isolated from LXRαβ^{-/-} mice have increased basal levels of ABCA1 compared to intestinal mucosa from wild type mice. This increase was not observed in any of the other tissues analyzed. These results show that LXR represses basal expression of ABCA1 in a tissue specific manner, occurring only in macrophage and intestinal mucosa. See FIG. 5.

### Example 4

LXR Interacts with the Co-Repressors NCoR and SMRT.

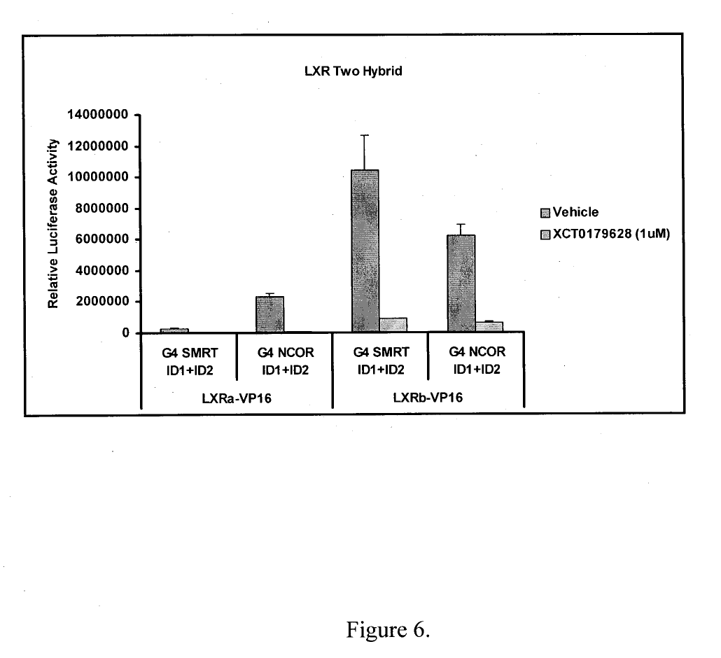

Nuclear receptors function as transcription factors by differentially recruiting other proteins known as co-factors to target gene promoters. In the absence of ligand some receptors have been shown to interact with co-repressors that inhibit transcription. To determine the mechanism by which LXR represses transcription we analyzed the ability of LXR to interact with the co-repressors NCoR (SEQ. ID. No. 15) and SMRT (SEQ. ID. No. 14). Two-hybrid analysis with Gal4 fusions of the receptor interacting domains of NCoR (SEQ. ID. No. 15) and SMRT (SEQ. ID. No. 14) and VP16 fusions of LXRα (SEQ. ID. No. 6) and LXRβ (SEQ. ID. No. 7) suggested that LXR interacts with the co-repressors in the absence of ligand and that this interaction is inhibited in the presence of LXR agonist. See FIG. 6.

### Example 5

LXR Represses Gal4 Basal Transcription.

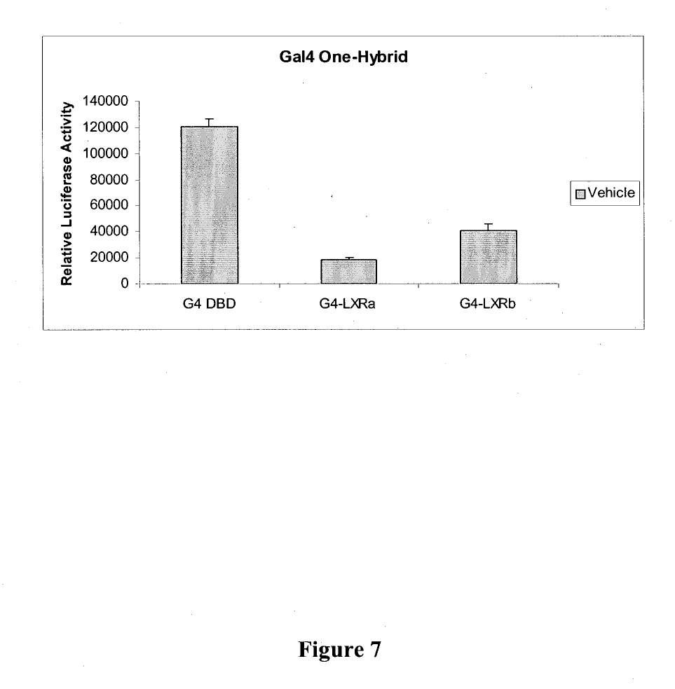

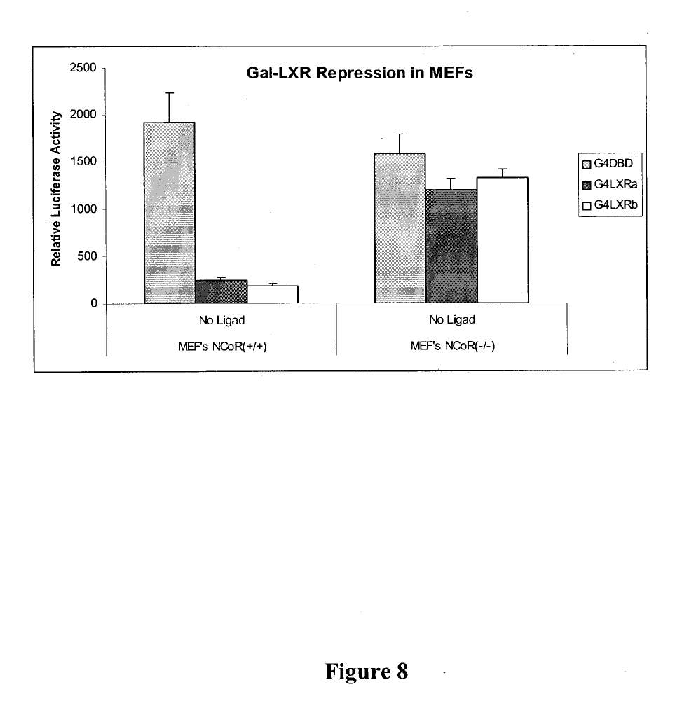

To determine if LXR can repress basal transcription in the absence of ligand we performed a one-hybrid experiment with Gal4-DBD-fusions of LXR. CV-1 cells were transfected with Gal-LXRα and Gal-LXRβ and a Gal4-Luciferase reporter. As shown above recruitment of LXR to the Gal4 promoter results in repression of basal transcription in the absence of ligand. To determine if NCoR (SEQ. ID. No. 15) mediates LXR repression of the Gal4 promoter we performed the same experiment in mouse embryonic fibroblasts isolated from wild type and NCoR knockout mice. As shown below, LXR is unable to repress Gal4 basal transcription in the absence of NcoR (SEQ. ID. No. 15), suggesting that association with the co-repressor is required for LXR mediated repression. See FIGS. 7 and 8.

### Example 6

High Throughput Co-Factor Recruitment Assays

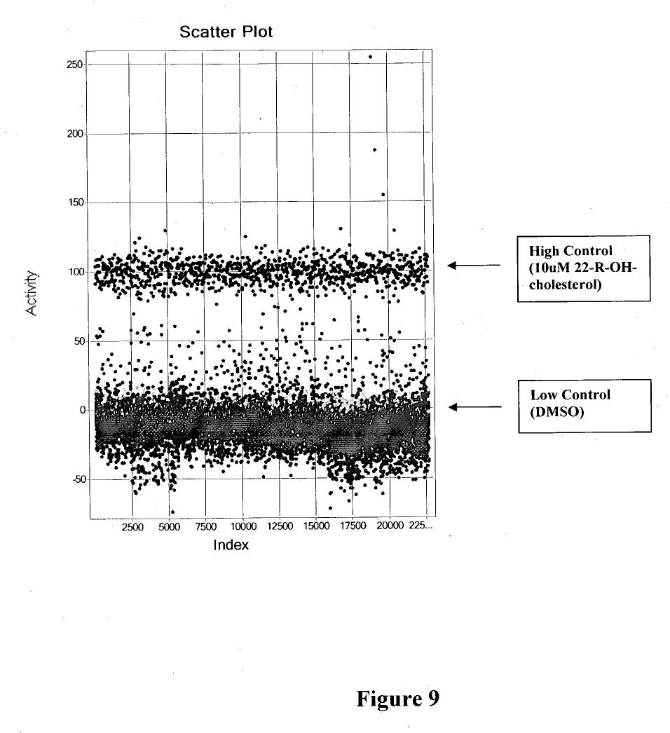

To identify test compounds that were able to recruit or disrupt the recruit of co-factors a high throughput FRET based co-activator recruitment screen was developed, validated and run with a 100,000 compounds. The FRET assay was validated with both LXRα (SEQ. ID. No. 6) and LXRβ (SEQ. ID. No. 6) and run in HTS mode as a multiplexed assay in which the recruitment of SCR-1 (SEQ. ID. No. 11) to both LXRα (SEQ. ID. No. 6) and LXRβ (SEQ. ID. No. 7) were simultaneously assayed. An example of a Spotfire visualization of the assay showing fluorescence emitted from APC at 665 nm is shown in FIG. 9. In this high throughput assay, the data centered around 100 are the positive controls (10 uM 22-R-Hydroxy-Cholesterol) data centered around 0 (barely visible) are the negative controls (DMSO) and the remaining data are assay data points.

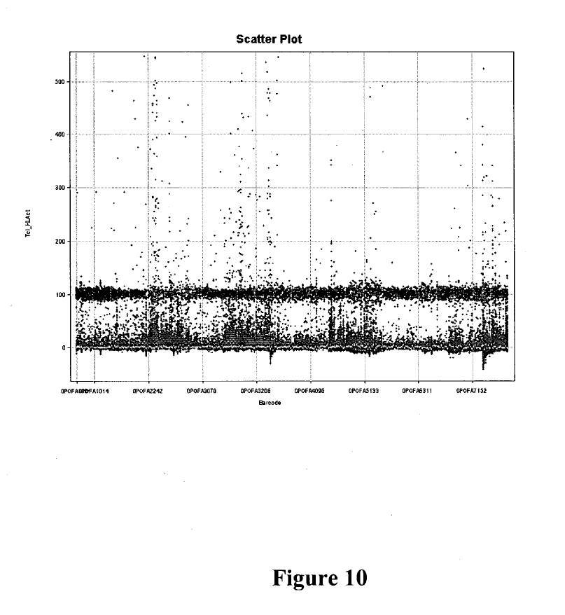

Data for the corresponding assay for recruitment of SCR-1 (SEQ. ID. No. 11) to FXR (SEQ. ID. No. 4) is shown in FIG. 10. In this case the screen was performed with approximately 20,000 compounds.

### Example 7

Mammalian Two-Hybrid Recruitment Assay

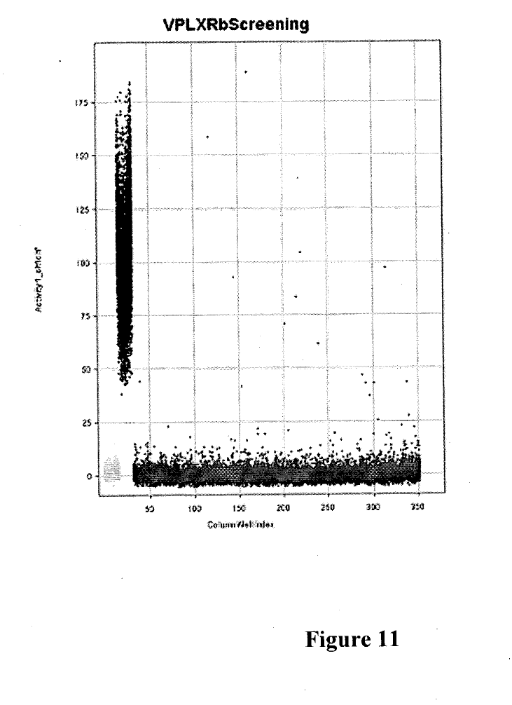

To identify test compounds that were able to recruit or disrupt the recruit of co-factors in situ, a cell based high throughput assay was developed, validated and run. The mammalian two hybrid assay was validated with both LXRα (SEQ. ID. No. 6) and LXRβ (SEQ. ID. No. 7) and run in HTS mode. A representative Spotfire visualization of a screen of 170,000 compounds using the mammalian two-hybrid assay with LXRβ (SEQ. ID. No. 7) is shown in FIG. 11. In this experiment, the positive controls centered around 100, and the negative control is centered around 0.

All of the U.S. patents, U.S. patent application publications, U.S. patent applications, foreign patents, foreign patent applications and non-patent publications referred to in this specification and/or listed in the Application Data sheets, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

The invention is further described below in the form of non-limiting enumerated embodiments:

We claim:

1. A method to identify compounds that bind to a nuclear receptor and exhibit cell type specific actions, said method comprising:
a) contacting a modified host cell with a test compound, wherein said modified host cell comprises:
i) a first fusion protein, comprising a co-activator fused to a first heterologous DNA binding domain,
ii) a second fusion protein comprising a co-repressor fused to a second heterologous DNA binding domain,
iii) a third fusion protein comprising a ligand binding domain of a nuclear receptor fused to a transcription activation domain,
iv) a first reporter gene operably linked to a first transcriptional regulatory sequence specific for said first heterologous DNA binding domain,
v) a second reporter gene operably linked to a second transcriptional regulatory sequence specific for said second heterologous DNA binding domain,
b) identifying those test compounds which cause altered expression of said first reporter gene product compared to a control modified host cell and similar, or altered expression of said second reporter gene product compared to said control modified host cell.

2. The method of claim 1, wherein said co-activator is selected from one of SEQ. ID. Nos. 47 to 85.

3. The method of claim 1, wherein said co-repressor is selected from one of SEQ. ID. Nos. 43, 44, 45 and 86.

4. The method of claim 1, wherein said co-activator is selected from the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39) and PGC-2 (SEQ. ID. No. 40).

5. The method of claim 1, wherein said co-repressor is selected from the group consisting of SMRT (SEQ. ID. No. 14), and N-CoR (SEQ. ID. No. 15).

6. The method of claim 1, wherein said first or said second heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16), wherein X can be any amino acid, Cys=cysteine, and His=histidine.

7. The method of claim 1, wherein said first or said second heterologous DNA binding domain is selected from the group consisting of a GAL4 DNA binding domain (SEQ. ID. No. 17), and a LexA DNA binding domain (SEQ. ID. No. 18),

8. The method of claim 1, wherein said first or said second heterologous DNA binding domain is selected from the group consisting of a GR DNA binding domain, an MR DNA binding domain, an AR DNA binding domain, a PR DNA binding domain and an ER DNA binding domain.

9. The method of claim 1, wherein said first or said second trans activation domain is selected from the group consisting of VP16 (SEQ. ID. No. 19), TAT (SEQ. ID. No. 20), and the GAL4 activation domain (SEQ. ID. No. 21).

10. The method of claim 1, wherein said nuclear receptor is a preferred nuclear receptor.

11. The method of claim 1, wherein said first or said second transcriptional regulatory sequence comprises the sequence -RGBNNM-(SEQ. ID. No. 22),
wherein R is selected from A or G; B is selected from G, C, or T; each N is independently selected from A, T, C or G and M is selected from A or C;
with the proviso that at least 4 nucleotides of said -RGBNNM-(SEQ. ID. No. 22) sequence are identical with the nucleotides at a corresponding position of the sequence -AGGTCA-(SEQ. ID. No. 23).

12. The method of claim 1, wherein said first or said second reporter gene is selected from the group consisting of luciferase, a naturally fluorescent protein, β-galactosidase, β-lactamase, alkaline phosphatase and chloramphenicol acetyltransferase.

13. The method of claim 1, wherein said test compound has a known Kd for said nuclear receptor of at least 500 nM.

14. A method to identify compounds that bind to a nuclear receptor and exhibit cell type specific actions, said method comprising:
a) contacting a first and second modified host cell with a test compound, wherein said first modified host cell comprises:
i) a first fusion protein, comprising a co-activator fused to a first heterologous DNA binding domain,
ii) a second fusion protein comprising a ligand binding domain of a nuclear receptor fused to a first transcription activation domain,
iii) a first reporter gene operably linked to a first transcriptional regulatory sequence specific for said first heterologous DNA binding domain, and
wherein said second modified host cell comprises,
i) a third fusion protein, comprising a co-repressor fused to said first heterologous DNA binding domain or a second heterologous DNA binding domain,
ii) a fourth fusion protein comprising said ligand binding domain of said nuclear receptor fused to said first transcription activation domain or a second transcription activation domain,
iii) a second reporter gene operably linked to said first transcriptional regulatory sequence specific for said first heterologous DNA binding domain or a second transcriptional regulatory sequence specific for said second heterologous DNA binding domain,
b)identifying those test compounds which cause altered expression of said first reporter gene product in said first modified host cell compared to a first modified host control cell, and similar or altered expression of said second reporter gene product in said second modified host cell, compared to a second modified host control cell.

15. The method of claim 14, wherein said co-activator is selected from one of SEQ. ID. Nos. 47 to 85.

16. The method of claim 14, wherein said co-repressor is selected from one of SEQ. ID. Nos. 43, 44, 45 and 86.

17. The method of claim 14, wherein said co-activator is selected from the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39) and PGC-2 (SEQ. ID. No. 40).

18. The method of claim 14, wherein said co-repressor is selected from the group consisting of SMRT (SEQ. ID. No. 14), and N-CoR (SEQ. ID. No. 15).

19. The method of claim 14, wherein said first or said second heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16), wherein X can be any amino acid, Cys=cysteine, and His=Histidine.

20. The method of claim 14, wherein said first or said second heterologous DNA binding domain is selected from the group consisting of a GAL4 DNA (SEQ. ID. No. 17) binding domain and a LexA DNA binding domain (SEQ. ID. No. 18).

21. The method of claim 14, wherein said first or said second heterologous DNA binding domain is selected from the group consisting of a GR DNA binding domain, an MR DNA binding domain, an AR DNA binding domain, a PR DNA binding domain and an ER DNA binding domain.

22. The method of claim 14, wherein said first or said second trans activation domain is selected from the group consisting of VP16 (SEQ. ID. No. 19), TAT (SEQ. ID. No. 20) and the GAL4 activation domain (SEQ. ID. No. 21).

23. The method of claim 14, wherein said nuclear receptor is a preferred nuclear receptor.

24. The method of claim 14, wherein said first or said second transcriptional regulatory sequence -comprises the sequence RGBNNM-(SEQ. ID. No. 22),
wherein R is selected from A or G; B is selected from G, C, or T; each N is independently selected from A, T, C or G and M is selected from A or C;
with the proviso that at least 4 nucleotides of said -RGBNNM-(SEQ. ID. No. 22) sequence are identical with the nucleotides at a corresponding position of the sequence -AGGTCA-(SEQ. ID. No. 23).

25. The method of claim 14, wherein said first or said second reporter gene is selected from the group consisting of luciferase, a naturally fluorescent protein, β-galactosidase, β-lactamase, alkaline phosphatase and chloramphenicol acetyltransferase.

26. The method of claim 14, wherein said test compound has a known Kd for said nuclear receptor of at least 500 nM.

27. A method to identify compounds to identify compounds that bind to a nuclear receptor and exhibit cell type specific actions, said method comprising:
a) contacting a modified host cell with a test compound, wherein said modified host cell comprises:
i) a first fusion protein, comprising a co-activator fused to a first heterologous DNA binding domain,
ii) a second fusion protein comprising a co-repressor fused to a second heterologous DNA binding domain,
iii) a third fusion protein comprising a ligand binding domain of a nuclear receptor fused to a transcription activation domain,
iv) a first reporter gene operably linked to a first transcriptional regulatory sequence specific for said first heterologous DNA binding domain,
v) a relay protein operably linked to a second transcriptional regulatory sequence specific for said second heterologous DNA binding domain,
vi) a second reporter gene operably linked to a third transcriptional regulatory sequence that is repressed by expression of said relay protein,
b) identifying those test compounds which caused altered expression of said first reporter gene product compared to a control modified host cell and similar or altered expression of said second reporter gene product compared to said control modified host cell.

28. The method of claim 27, wherein said co-activator is selected from one of SEQ. ID. Nos.47 to 85.

29. The method of claim 27, wherein said co-repressor is selected from one of SEQ. ID. Nos. 43, 44, 45 and 86.

30. The method of claim 27, wherein said co-activator is selected from the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39) and PGC-2 (SEQ. ID. No. 40).

31. The method of claim 27, wherein said co-repressor is selected from the group consisting of SMRT (SEQ. ID. No. 14), and N-CoR (SEQ. ID. No. 15).

32. The method of claim 27, wherein said first, said second heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16), wherein X can be any amino acid, Cys=cysteine, and His=Histidine.

33. The method of claim 27, wherein said first or said second heterologous DNA binding domain is selected from the group consisting GAL4 DNA binding domain (SEQ. ID. No. 17) and a LexA DNA binding domain (SEQ. ID. No. 18).

34. The method of claim 27, wherein said first or said second heterologous DNA binding domain is selected from the group consisting of a GR DNA binding domain, an MR DNA binding domain, an AR DNA binding domain, a PR DNA binding domain and an ER DNA binding domain.

35. The method of claim 27, wherein said first or said second trans activation domain is selected from the group consisting of VP16 (SEQ. ID. No. 19), TAT (SEQ. ID. No. 20) and the GAL4 activation domain (SEQ. ID. No. 21).

36. The method of claim 27, wherein said nuclear receptor is a preferred nuclear receptor.

37. The method of claim 27, wherein said first, said second or said third transcriptional regulatory sequence comprises the sequence -RGBNNM-(SEQ. ID. No. 22),
wherein R is selected from A or G; B is selected from G, C, or T; each N is independently selected from A, T, C or G and M is selected from A or C;
with the proviso that at least 4 nucleotides of said RGBNNM-(SEQ. ID. No. 22) sequence are identical with the nucleotides at a corresponding position of the sequence -AGGTCA-(SEQ. ID. No. 23).

38. The method of claim 27, wherein said first or said second reporter gene is selected from the group consisting of luciferase, a naturally fluorescent protein, β-galactosidase, β-lactamase, alkaline phosphatase and chloramphenicol acetyltransferase.

39. The method of claim 27, wherein said test compound has a known Kd for said nuclear receptor of at least 500 nM.

40. A method to identify compounds that bind to a nuclear receptor and exhibit cell type specific actions, said method comprising:
a) contacting a first and second modified host cell with a test compound,
wherein said first modified host cell comprises:
i) a first fusion protein, comprising a co-activator fused to a first heterologous DNA binding domain,
ii) a second fusion protein comprising a ligand binding domain of a nuclear receptor fused to a first transcription activation domain,
iii) a first reporter gene operably linked to a first transcriptional regulatory sequence specific for said first heterologous DNA binding domain, and
wherein said second modified host cell comprises:
vi) a third fusion protein, comprising a co-repressor fused to said first heterologous binding domain or a second heterologous binding domain,
vii) a fourth fusion protein, comprising said ligand binding domain fused to said first transcription activation domain or a second transcription activation domain,
viii) a relay plasmid comprising DNA encoding a relay protein operably linked to said first transcriptional regulatory sequence specific for said first heterologous DNA binding domain or a second transcriptional regulatory sequence specific for said second heterologous DNA binding domain,
ix) a reporter gene operably linked to a third transcriptional regulatory sequence that is repressed by expression of said relay protein,
b) identifying those test compounds which cause altered expression of said first reporter gene product in said first modified host cell compared to a first control modified host cell, and similar or altered expression of said second reporter gene product in said second modified host cell compared to a second control modified host cell.

41. The method of claim 40, wherein said co-activator is selected from one of SEQ. ID. Nos. 47 to 85.

42. The method of claim 40, wherein said co-repressor is selected from one of SEQ. ID. Nos. 43, 44, 45 and 86.

43. The method of claim 40, wherein said co-activator is selected from the group consisting of SRC-1 (SEQ. ID. No. 11), TIF2 (SEQ. ID. No. 13), p/CIP (SEQ. ID. No. 35), TRAP250 (SEQ. ID. No. 12), PGC-1 (SEQ. ID. No. 39) and PGC-2 (SEQ. ID. No. 40).

44. The method of claim 40, wherein said co-repressor is selected from the group consisting of SMRT (SEQ. ID. No. 14), and N-CoR (SEQ. ID. No. 15).

45. The method of claim 40, wherein said first or said second heterologous DNA binding domain comprises a zinc finger motif of general formula X-X-Cys-X₍₁₋₅₎-Cys-X-X-X-X-X-X-X-X-X-X-X-X-His-X₍₃₋₆₎-[His/Cys] (SEQ. ID. No. 16), wherein X can be any amino acid, Cys=cysteine, and His=Histidine.

46. The method of claim 40, wherein said first or said second heterologous DNA binding domain is selected from the group consisting of a GAL4 DNA binding domain (SEQ. ID. No. 17) and a LexA DNA binding domain (SEQ. ID. No. 18).

47. The method of claim 40, wherein said first or said second heterologous DNA binding domain is selected from the group consisting of a GR DNA binding domain, an MR DNA binding domain, an AR DNA binding domain, a PR DNA binding domain and an ER DNA binding domain.

48. The method of claim 40, wherein said first or said second trans activation domain is selected from the group consisting of VP16 (SEQ. ID. No. 19), TAT (SEQ. ID. No. 20) and the GAL4 activation domain (SEQ. ID. No. 21).

49. The method of claim 40, wherein said nuclear receptor is a preferred nuclear receptor.

50. The method of claim 40, wherein said first or said second transcriptional regulatory sequence comprises the sequence -RGBNNM-(SEQ. ID. No. 22),
wherein R is selected from A or G; B is selected from G, C, or T; each N is independently selected from A, T, C or G and M is selected from A or C;
with the proviso that at least 4 nucleotides of said -RGBNNM-(SEQ. ID. No. 22) sequence are identical with the nucleotides at a corresponding position of the sequence -AGGTCA-(SEQ. ID. No. 23).

51. The method of claim 40, wherein said first or said second reporter gene is selected from the group consisting of luciferase, a naturally fluorescent protein, β-galactosidase, β-lactamase, alkaline phosphatase and chloramphenicol acetyltransferase.

52. The method of claim 40, wherein said test compound has a known Kd for said nuclear receptor of at least 500 nM.

53. A method to identify compounds that bind to a nuclear receptor and exhibit cell type specific actions, said method comprising:
a) providing a composition comprising,
i) an affinity support, comprising a first fusion protein comprising a ligand binding domain of a nuclear receptor fused to an affinity tag that couples said first fusion protein to said affinity support,
ii) a second fusion protein, comprising a co-activator coupled to a first detectable label,
iii) a third fusion protein comprising a co-repressor coupled to a second detectable label,
b) incubating said composition in an aqueous buffer comprising a test compound,
c) detecting the binding of said co-activator and said co-repressor to said first fusion protein,
d) selecting compounds that cause disrupted, or substantially disrupted binding of said co-repressor without increasing binding of said co-activator to said ligand binding domain compared to a control composition.

54. The method of claim 53, wherein said co-activator is selected from one of SEQ. ID. Nos. 47 to 85.

55. The method of claim 53, wherein said co-repressor is selected from one of SEQ. ID. Nos. 43, 44, 45 and 86.

56. The method of claim 53, wherein said first or said second detectable label is selected from the group consisting of a radiolabel, affinity tag, a fluorescent or luminescent label and an enzymatic label.

57. The method of claim 53, wherein said radiolabel is selected from the group consisting of ³H, ¹⁴C, ³⁵S, ¹²⁵I, and ¹³¹I.

58. The method of claim 53, wherein said affinity tag is selected from the group consisting of biotin, a binding sites for an antibody, a metal binding domain, a FLASH binding domain, and a GST binding domain.

59. The method of claim 53, wherein said enzymatic label is selected from the group consisting of horseradish peroxidase, β-galactosidase, β-lactamase, luciferase and alkaline phosphatase.

60. The method of claim 53, wherein said fluorescent or luminescent label is selected from the group consisting of fluorescein, a naturally fluorescent protein, rhodamine, and a lanthanide.

61. A method to identify compounds that bind to a nuclear receptor and exhibit cell type specific actions, said method comprising,
a) providing a compositions comprising;
i) a ligand binding domain of a nuclear receptor, and
ii) a co-activator coupled to a first detectable label, and
iii) a co-repressor coupled to a second detectable label,
b) incubating said composition in an aqueous buffer comprising a test compound,
c) detecting binding of said co-activator and co-repressor with said ligand binding domain, and
d) selecting compounds that cause disrupted, or substantially disrupted binding of said co-repressor without increasing binding of said co-activator to said ligand binding domain compared to a control composition.

62. The method of claim 61, wherein said co-activator is selected from one of SEQ. ID. Nos. 47 to 85.

63. The method of claim 61, wherein said co-repressor is selected from one of SEQ. ID. Nos. 43, 44, 45 and 86.

64. The method of claim 61, wherein said first or said second detectable label is selected from the group consisting of a radiolabel, affinity tag, a fluorescent or luminescent label and an enzymatic label.

65. The method of claim 61, wherein said radiolabel is selected from the group consisting of ³H, ¹⁴C, ³⁵S, ¹²⁵I, and ¹³¹I.

66. The method of claim 61, wherein said affinity tag is selected from the group consisting of biotin, a binding sites for an antibody, a metal binding domain, a FLASH binding domain, and a GST binding domain.

67. The method of claim 61, wherein said enzymatic label is selected from the group consisting of horseradish peroxidase, β-galactosidase, β-lactamase, luciferase and alkaline phosphatase.

68. The method of claim 61, wherein said fluorescent or luminescent label is selected from the group consisting of fluorescein, a naturally fluorescent protein, rhodamine, and a lanthanide.

69. A method to identify compounds that bind to a nuclear receptor and exhibit cell type specific actions, said method comprising,
a) providing first and second compositions, wherein said first composition comprises;
i) a ligand binding domain of a nuclear receptor coupled to a first detectable label, and
ii) a co-activator coupled to a second detectable label, and
wherein said second composition comprises;
iii) said ligand binding domain, coupled to said first detectable label, and
iv) a co-repressor coupled to said second detectable label,
b) incubating said first composition and said second composition in an aqueous buffer comprising a test compound,
c) detecting the binding of said co-activator with said ligand binding domain in said first composition and detecting the binding of said co-repressor with said ligand binding domain in said second composition,
d) selecting compounds that cause disrupted, or substantially disrupted binding of said co-repressor compared to a first control composition, and failed to increase binding of said co-activator to said ligand binding domain compared to a second control composition.

70. The method of claim 69, wherein said co-activator is selected from one of SEQ. ID. Nos. 47 to 85.

71. The method of claim 69, wherein said co-repressor is selected from one of SEQ. ID. Nos. 43, 44, 45 and 86.

72. The method of claim 69, wherein said first or said second detectable label is selected from the group consisting of a radiolabel, affinity tag, a fluorescent or luminescent label and an enzymatic label.

73. The method of claim 69, wherein said radiolabel is selected from the group consisting of ³H, ¹⁴C, ³⁵S, ¹²⁵I, and ¹³¹I.

74. The method of claim 69, wherein said affinity tag is selected from the group consisting of biotin, a binding sites for an antibody, a metal binding domain, a FLASH binding domain, and a GST binding domain.

75. The method of claim 69, wherein said enzymatic label is selected from the group consisting of horseradish peroxidase, β-galactosidase, β-lactamase, luciferase and alkaline phosphatase.

76. The method of claim 69, wherein said fluorescent or luminescent label is selected from the group consisting of fluorescein, a naturally fluorescent protein, rhodamine, and a lanthanide.
